(19) United States
(12) Patent Application Publication
(10) Pub. No.: US 2022/0289822 A1
(43) Pub. Date: Sep. 15, 2022

(54) NOVEL IL-21 PRODRUGS AND METHODS OF USE THEREOF

(71) Applicant: ASKGENE PHARMA, INC., Camarillo, CA (US)

(72) Inventors: Yuefeng LU, Moorpark, CA (US); Chunxiao YU, Santa Barbara, CA (US); Liqin LIU, Woodland Hills, CA (US); Jian-Feng (Jeff) LU, Oak Park, CA (US); Jui Chang (Ray) CHUANG, Camarillo, CA (US)

(73) Assignee: ASKGENE PHARMA, INC., Camarillo, CA (US)

(21) Appl. No.: 17/635,683

(22) PCT Filed: Aug. 21, 2020
(86) PCT No.: PCT/US2020/047522

§ 371 Date: Feb. 15, 2022

Related U.S. Application Data
(60) Provisional application No. 62/889,797, filed on Aug. 21, 2019.
(60) Provisional application No. 63/027,138, filed on May 19, 2020.
(60) Provisional application No. 63/047,251, filed on Jul. 1, 2020.
(60) Provisional application No. 63/053,663, filed on Jul. 19, 2020.

(51) Int. Cl. C07K 14/705; C07K 14/54; C07K 14/55; C07K 14/715; C07K 16/24; A61P 35/00

(52) U.S. Cl. CPC C07K 14/70596; C07K 14/5443; C07K 14/55; C07K 14/70578; C07K 14/7155; C07K 16/244; A61P 35/00; C07K 2317/76; C07K 2319/31; C07K 2319/50

(57) ABSTRACT

Provided herein are IL-21 prodrugs and methods of making and using thereof for stimulating the immune system, or treating cancer or an infectious disease.

### CROSS REFERENCE TO RELATED APPLICATIONS

The present application claims priority from U.S. Provisional Application No. 62/889,797, filed on Aug. 21, 2019; U.S. Provisional Application No. 63/027,138, filed on May 19, 2020; U.S. Provisional Application No. 63/047,251, filed on Jul. 1, 2020; and U.S. Provisional Application No. 63/053,663, filed on Jul. 19, 2020, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Aug. 18, 2020, is named 025471_WO006_SL.txt and is 401,040 bytes in size.

### BACKGROUND OF THE INVENTION

Interleukin-21 (IL-21) is produced by activated CD4⁺ T cells, T-follicular helper cells, and natural killer T (NKT) cells (Spolski and Leonard, Ann Rev Immunol. (2008) 26:57008). IL-21 has been shown to exert pleiotropic effects on the proliferation, differentiation, and cytotoxicity of various classes of lymphoid cells. More recently, IL-21 has been further shown to play a crucial role in the differentiation of CD4⁺ T cells into T-helper 17 (TH₁₇) cells, a subset of T cells associated with development of inflammatory conditions and autoimmune diseases (Korn et al., Nature (2007) 448(7152):484-87; Nurieva et al., Nature (2007) 448(7152):480-83).

The receptor complex of IL-21 is composed of the private chain IL-21Rα and the common chain γC (or Rγ); the common chain is shared by five other cytokines: IL-2, IL-4, IL-7, IL-9, and IL-15 (Spolski and Leonard, supra). Human IL-21 binds to IL-21Rα with a very high affinity (K_{D}˜70 pM; Zhang et al., Biochem Biophys Res Commun. (2003) 300(2):291-6), while binding to IL-21Rγ with a relatively low affinity (K_{D}˜160 μM).

Recombinant IL-21 has been studied in several clinical trials for the treatment of solid tumors (Zarkavelis et al., Transl Cancer Res. (2017) 6(Suppl 2):S328-30). In one of the studies, the maximum tolerated dose was established at 200 μg/kg (Schmidt et al., Clin Cancer Res. (2010) 16(21):5312-19). Thus, as with other cytokine therapies, systemic toxicity may severely limit the therapeutic dosage of IL-21. In addition, IL-21 may encounter “PK sinks” in vivo because it binds to its receptor IL-21Rα with very high affinity (K_{D} of about 70 pM) (Zhang et al., Biochem Biophys Res Commun. (2003) 300(2):291-6). Consequently, it may be difficulty for IL-21 to achieve optimal PK and exposure in cancer treatment. Analogs of IL-21 have been disclosed in U.S. Pat. No. 8,211,420 and Kan et al., J Biol Chem. (2010) 285(16):12223-31. However, some of the analogs have selectively reduced γC binding affinity and are IL-21 antagonists.

There remains a need to develop IL-21-based cancer therapeutics that are more tumor site-selective and have improved PK and efficacy, while causing fewer severe side effects.

### SUMMARY OF THE INVENTION

The present disclosure provides a IL-21 prodrug comprising a cytokine moiety, a masking moiety, and a carrier moiety, wherein the cytokine moiety is an IL-21 agonist polypeptide, wherein the masking moiety comprises an antigen-binding fragment of an antibody that binds to the human IL-21 agonist polypeptide and inhibits a biological activity of the human IL-21 agonist polypeptide, and wherein the IL-21 agonist polypeptie is fused to the carrier moiety and the masking moiety is fused to the human IL-21 agonist polypeptide or to the carrier moiety, optionally through a peptide linker.

In some embodiments, the cytokine moiety is a wildtype human IL-21 or a mutein thereof, for example, the human IL-21 agonist polypeptide such as one comprising SEQ ID NO: 1 or an amino acid sequence that is at least 90% identical to SEQ ID NO: 1. In other embodiments, the human IL-21 agonist polypeptide has an amino acid sequence selected from SEQ ID NO: 2, 3, 4, and 5.

In some embodiments, the masking moiety of the present prodrug comprises a binding fragment of an antibody which binds to the IL-21 agonist polypeptide; and wherein the antibody inhibits the binding of the IL-21 agonist polypeptide to an IL-21 receptor. In some embodiments, the antigen binding moiety is the binding fragment of an antibody against human IL-21 and comprises a heavy chain variable domain with an amino acid sequence at least 95% identical as that of SEQ ID NO: 97 or 99, and a light chain variable domain with an amino acid sequence at least 95% identical as that of SEQ ID NO: 98 or 100.

In some embodiments, the antibody fragment is a single chain fragment variable (scFv) comprising a heavy chain variable domain with an amino acid sequence as shown in SEQ ID NO: 97 and a light chain variable domain with an amino acid sequence as shown in SEQ ID NO: 98, or a heavy chain variable domain with an amino acid sequence as shown SEQ ID NO: 99 and a light chain variable domain with an amino acid sequence as shown in SEQ ID NO: 100.

In some embodiments of the present prodrugs, the cytokine moiety is fused to the carrier moiety through a non-cleavable peptide linker selected from SEQ ID NOs: 29-33 ad 132. In other embodiments, the masking moiety is fused to the carrier moiety or the cytokine moiety through a non-cleavable peptide linker, such as one selected from SEQ ID NOs: 29-33 and 132.

In some embodiments of the present prodrugs, the cleavable peptide linker linking the masking moiety directly or indirectly (e.g., through the cytokine moiety) to the carrier moiety comprises a substrate sequence of urokinase-type plasminogen activator (uPA), matriptase, matrix metallopeptidase (MMP) 2, or MMP9. In further embodiments, the cleavable peptide linker comprises substrate sequences of (i) both uPA and MMP2, (ii) both uPA and MMP9, or (iii) matriptase, MMP2 and MMP9. In particular embodiments, the cleavable peptide linker comprises an amino acid sequence selected from SEQ ID NOs: 11-26. In certain embodiments, the cleavable peptide linker is cleavable by one or more proteases located at a tumor site or its surrounding environment, and the cleavage leads to activation of the prodrug at the tumor site or surrounding environment.

In some embodiments of the present prodrugs, the carrier moiety is an antibody Fc domain, or an antibody or an antigen-binding fragment thereof. In particular embodiments, the carrier moiety is an IgG1 antibody Fc domain or an antibody that comprises mutations L234A and L235A (“LALA”) (EU numbering) or an IgG4 Fc domain that comprises mutations S228P/L234A/L235A (PAA). Other mutations which lead to the reduced Fc functionality such as the ones described by Tam S. H., et al. Antibodies (2017), 6(12): 1-34, can also be introduced when the Fc domain or an antibody is used as the carrier moiety.

In particular embodiments, the carrier moiety is an antibody Fc domain or an antibody, wherein the cytokine moiety and the masking moiety are fused to different polypeptide chains of the antibody Fc domain or to the different heavy chains of the antibody. In some embodiments, the cytokine moiety and the masking moiety are fused to the C-termini of the two different polypeptide chains of the Fc domain or to the C-termini of the two different heavy chains of the antibody. In other embodiments, the cytokine moiety and the masking moiety are fused to the N-termini of the two different polypeptide chains of the Fc domain or to the N-termini of the two different heavy chains of the antibody. In some embodiments, the carrier moiety is an antibody Fc domain or an antibody comprising knobs-into-holes mutations. In certain embodiments, the knobs-into-holes mutations comprise a T366Y “knob” mutation on a polypeptide chain of the Fc domain or a heavy chain of the antibody, and a Y407T “hole” mutation in the other polypeptide of the Fc domain or the other heavy chain of the antibody (EU numbering). In certain embodiments, the knobs-into-holes mutations comprise Y349C and/or T366W mutations in the CH3 domain of the “knob chain” and E356C, T366S, L368A, and/or Y407V mutations in the CH3 domain of the “hole chain” (EU numbering).

In particular embodiments, the prodrug comprises two polypeptide chains whose amino acid sequences respectively comprise

SEQ ID NO: 36 and one selected from SEQ ID NO: 101-104,

SEQ ID NO: 37 and one selected from SEQ ID NO: 101-104,

SEQ ID NO: 39 and one selected from SEQ ID NO: 105-108, or

SEQ ID NO: 40 and one selected from SEQ ID NO: 105-108,

SEQ ID NO: 42 and one selected from SEQ ID: 113-116, or

SEQ ID NO: 43 and one selected from SEQ ID NO: 113-116.

In some embodiments, the carrier moiety is an antibody or an antigen-binding fragment thereof that specifically binds to one or more antigens selected from guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, CD47, signal-regulatory protein alpha (SIRPα), PD1, Claudin 18.2, Claudin 6, 5T4, BCMA, PD-L1, PD-1, fibroblast activation protein alpha (FAPalpha), the melanoma-associated chondroitin sulfate proteoglycan (MCSP), and epithelial cellular adhesion molecule (EPCAM). In specific embodiments, the carrier moiety is an antibody or fragment thereof which binds to FAPalpha or 5T4.

In particular embodiments, the carrier moiety is an antibody, wherein the prodrug comprises two identical light chains and two heavy chain polypeptides; wherein the light chain comprises an amino acid sequence as shown in SEQ ID NO: 50 or 51; and wherein the first heavy chain polypeptide chain comprises SEQ ID NO: 48, and the second heavy chain polypeptide chain comprises an amino acid sequence selected from SEQ ID NO: 109-112.

In particular embodiments, the carrier moiety is an antibody comprising one antigen-binding domain, wherein the prodrug comprises one Fc fusion polypeptide, one light chain and one heavy chain polypeptide chain; wherein the Fc fusion polypeptide comprises an amino acid sequence selected from SEQ ID NO: 101-104; the light chain comprises an amino acid sequence as shown in SEQ ID NO: 50 or 51; and the heavy chain polypeptide chain comprises SEQ ID NO: 48.

In particular embodiments, the carrier moiety is an antibody comprising one antigen-binding domain, wherein the prodrug comprises one Fc fusion polypeptide, one light chain and one heavy chain polypeptide chain; wherein the Fc fusion polypeptide comprises an amino acid sequence selected from SEQ ID NO: 36 and 37; the light chain comprises an amino acid sequence as shown in SEQ ID NO: 50 or 51; and the heavy chain polypeptide chain comprises an amino acid sequence selected from SEQ ID NO: 109-112.

In some embodiments, the prodrug further comprises an extracellular domain (ECD) of IL-21 receptor, wherein the ECD comprises an amino acid sequence of SEQ ID NO: 128, or at least 95% identical as that of SEQ ID NO: 128.

In some embodiments, the prodrug comprises a light chain, a first heavy chain polypeptide chain and a second heavy chain polypeptide chain; wherein the light chain comprises an amino acid sequence of SEQ ID NO: 50 or at least 95% identical as SEQ ID NO: 50, the first heavy chain polypeptide chain comprises an amino acid sequence of SEQ ID NO: 117 or 129, or at least 95% identical as that of SEQ ID NO: 117 or 129, and the second heavy chain polypeptide chain with an amino acid sequence selected from SEQ ID NOs: 120, 121, 124, 125, 130, and 131, or an amino acid sequence at least 95% identical as one selected from SEQ ID NOs: 120, 121, 124, 125, 130, and 131.

In some embodiments, the prodrug comprises a light chain, a first heavy chain polypeptide chain and a second heavy chain polypeptide chain; wherein the light chain comprises an amino acid sequence of SEQ ID NO: 50 or at least 95% identical as SEQ ID NO: 50, the first heavy chain polypeptide chain comprises an amino acid sequence of SEQ ID NO: 118 or at least 95% identical as that of SEQ ID NO: 118, and the second heavy chain polypeptide chain with an amino acid sequence selected from SEQ ID NOs: 122 and 126, or an amino acid sequence at least 95% identical as one selected from SEQ ID NOs: 122 and 126.

In some embodiments, the prodrug comprises a light chain, a first heavy chain polypeptide chain and a second heavy chain polypeptide chain; wherein the light chain comprises an amino acid sequence of SEQ ID NO: 50 or at least 95% identical as SEQ ID NO: 50, the first heavy chain polypeptide chain comprises an amino acid sequence of SEQ ID NO: 119 or at least 95% identical as that of SEQ ID NO: 119, and the second heavy chain polypeptide chain with an amino acid sequence selected from SEQ ID NOs: 123 and 127, or an amino acid sequence at least 95% identical as one selected from SEQ ID NOs: 123 and 127.

In some embodiments, the prodrug comprises a light chain, a first heavy chain polypeptide chain and a second heavy chain polypeptide chain; wherein the light chain comprises an amino acid sequence of SEQ ID NO: 50 or at least 95% identical as SEQ ID NO: 50, the first heavy chain polypeptide chain comprises an amino acid sequence of SEQ ID NO: 117 or 129, or at least 95% identical as that of SEQ ID NO: 117 or 129, and the second heavy chain polypeptide chain with an amino acid sequence selected from SEQ ID NOs: 120, 121, 124, 125, 130, and 131, or an amino acid sequence at least 95% identical as one selected from SEQ ID NOs: 120, 121, 124, 125, 130, and 131.

In other aspects, the present disclosure provides also a pharmaceutical composition comprising the IL-21 prodrug of the present disclosure and a pharmaceutically acceptable excipient; a polynucleotide or polynucleotides encoding the IL-21 prodrug, an expression vector or vectors comprising the polynucleotide or polynucleotides; and a host cell comprising the vector(s), wherein the host cell may be a prokaryotic cell or a eukaryotic cell such as a mammalian cell. In some embodiments, the mammalian host cell has the gene or genes encoding uPA, MMP-2 and/or MMP-9 knocked out (e.g., containing null mutations of one or more of these genes). Accordingly, the present disclosure also provides a method of making the IL-21 prodrug, comprising culturing the host cell under conditions that allow expression of the IL-21 prodrug, wherein the host cell is a mammalian cell, and isolating the IL-21 prodrug.

The present disclosure also provides a method of treating a cancer or an infectious disease or stimulating the immune system in a patient (e.g., human patient) in need thereof, comprising administering to the patient a therapeutically effective amount of the IL-21 prodrug, or the pharmaceutical composition of the present disclosure. The patient may have, for example, a viral infection (e.g., HIV infection), or a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer. Also provided herein are an IL-21 prodrug for use in treating a cancer or an infectious disease or stimulating the immune system in the present method; use of an 11-21 prodrug for the manufacture of a medicament for treating a cancer or an infectious disease or stimulating the immune system in the present method; and articles of manufacture (e.g., kits) comprising one or more dosing units of the present 11-21 prodrug.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTIONS OF THE DRAWINGS

FIG. 1 illustrates a heterodimeric IL-21 prodrug wherein the carrier is a Fc domain. The two chains of the Fc domain contain knobs-into-holes mutations.

FIG. 2 illustrates a tetrameric IL-21 prodrug wherein the carrier is an antibody containing knobs-into-holes mutations in the Fc domain.

FIG. 3A illustrates a heterodimeric IL-21 prodrug comprising (i) an IL-21 polypeptide and its corresponding mask, and (ii) an IL-2 mutein and its corresponding mask.

FIG. 3B illustrates an IL-21 prodrug comprising (i) an IL-21 polypeptide and its corresponding mask, and (ii) an IL-15 polypeptide, an IL-15Rα sushi domain and the corresponding mask.

FIG. 4 illustrates an IL-21 prodrug with two 4-1BBL ectodomains.

FIGS. 5A-5C show the size exclusion chromatography (SEC) HPLC analysis of the IL-21 Prodrug A after purification.

FIG. 6 shows the SDS-PAGE analysis of the IL-21 prodrugs prior to and after activation by the protease matrix metalloproteinase-2 (MMP2). Prodrug A comprises a wild type IL-21 polypeptide; while Prodrug B comprises an IL-21 mutein with mutations Q19K/E109R.

FIGS. 7A and 7B show the results of a cell-based biological activity assay of IL-21 prodrugs prior to and after activation by protease MMP2. FIG. 7A shows the results of the IL-21 Prodrug A, which comprises wild type IL-21. FIG. 7B shows the results of the IL-21 Prodrug B, which also comprise an IL-21 mutein.

FIG. 8 shows the results of a cell-based biological activity assay of PD-1-IL-21 Prodrugs prior to and after activation by protease MMP2.

FIG. 9 shows the results of a PD-1 reporter assay, which shows the ability of the anti-PD-1 antibody of the fusion molecules to block PD-L1-mediated PD-1 signaling.

FIG. 10 shows the binding of the IL-21 prodrugs and control molecules to the Mino cells. The binding was analyzed by FACS. Both the PD-1 antibody and the Fc-IL-21 fusion molecule showed binding to the Mino cells, indicating that Mino cells expressed both PD-1 and IL-21 receptor(s). The results further show that the Fc-based prodrugs did not bind to the Mino cell well.

FIG. 11 shows the results of a NK-92 cell-based biological activity assay of IL-21 prodrugs prior to and after activation by protease MMP2 and the control molecules. The results show that the prodrugs, especially the one masked with IL-21Rα-ECD had very low activity prior to activation. PW04-38 IL21 wt/alpha is an anti-PD-1 antibody-based IL-21 with IL-21R α-ECD as the masking moiety; PW05-68 is a PD-1 antibody-based IL-21 prodrug with the scFv as the masking moiety. A first control molecule PW05-67 IL21vQ116Y is an anti-PD-1 antibody-IL-21 fusion molecule without a mask and having an IL-21 mutein with a Q116Y amino acid substitution (numbering according to SEQ ID NO: 1). Another control molecule PW04-67 IL21 wt is an anti-PD-1 antibody-IL-21 fusion molecule without a mask and having a wild-type IL-21. Another control molecule, JR5.2.2 IL21R9ER76A is an anti-PD-1 antibody-IL-21 fusion molecule without a mask and with an IL-21 mutein with R9E and R76A amino acid substitutions (numbering according to SEQ ID NO: 1). PW04-38 act. IL21 wt is an anti-PD-1 antibody-IL-21 wild type fusion molecule whose mask has been cleaved with a protease.

FIGS. 12A and 12B show the results of the Mino cell-based biological activity assay of PD-1-IL-21 Prodrugs prior to and after activation by protease MMP2 and the control molecules. FIG. 12A shows the results after 72 hours of incubation of the cytokine fusion molecules with the Mino cells prior to the analysis. FIG. 12B shows the results after 120 hours of incubation prior to the analysis.

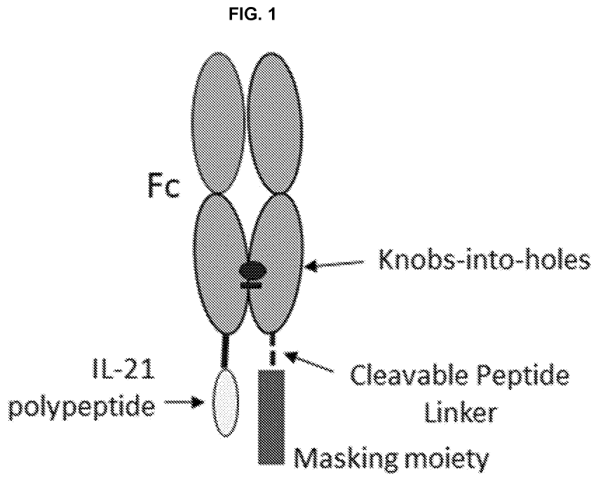

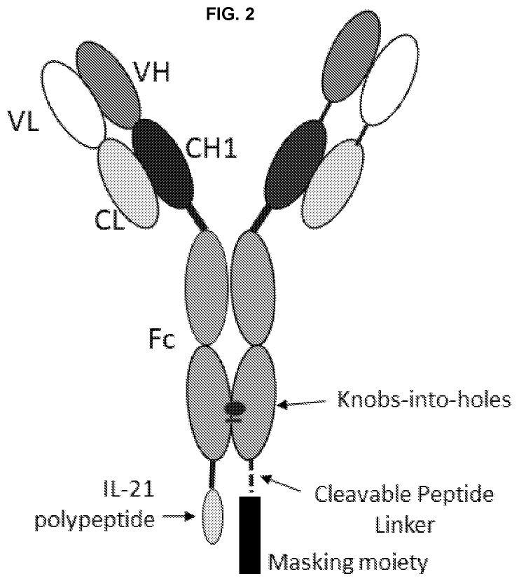

### DETAILED DESCRIPTION OF THE INVENTION

As used herein and in the appended claims, the singular forms “a,” “or,” and “the” include plural referents unless the context clearly dictates otherwise.

Reference to “about” a value or parameter herein includes (and describes) variations that are directed to that value or parameter per se. For example, description referring to “about X” includes description of “X.” Additionally, use of “about” preceding any series of numbers includes “about” each of the recited numbers in that series. For example, description referring to “about X, Y, or Z” is intended to describe “about X, about Y, or about Z.”

The term “antigen-binding moiety” refers to a polypeptide or a set of interacting polypeptides that specifically bind to an antigen, and includes, but is not limited to, an antibody (e.g., a monoclonal antibody, polyclonal antibody, a multi-specific antibody, a dual specific or bispecific antibody, an anti-idiotypic antibody, or a bifunctional hybrid antibody) or an antigen-binding fragment thereof (e.g., a Fab, a Fab′, a F(ab′)₂, a Fv, a disulfide linked Fv, a scFv, a single domain antibody (dAb), or a diabody, a single chain antibody, and an Fc-containing polypeptide such as an immunoadhesin. In some embodiments, the antibody may be of any heavy chain isotype (e.g., IgG, IgA, IgM, IgE, or IgD) or subtype (e.g., IgG₁, IgG₂, IgG₃, or IgG₄). In some embodiments, the antibody may be of any light chain isotype (e.g., kappa or lambda). The antibody may be human, non-human (e.g., from mouse, rat, rabbit, goat, or another non-human animal), chimeric (e.g., with a non-human variable region and a human constant region), or humanized (e.g., with non-human CDRs and human framework and constant regions). In some embodiments, the antibody is a derivatized antibody.

The term “cytokine agonist polypeptide” refers to a wildtype cytokine, or an analog thereof. An analog of a wildtype cytokine has the same biological specificity (e.g., binding to the same receptor(s) and activating the same target cells) as the wildtype cytokine, although the activity level of the analog may be different from that of the wildtype cytokine. The analog may be, for example, a mutein (i.e., mutated polypeptide) of the wildtype cytokine, and may comprise at least one, at least two, at least three, at least four, at least five, at least six, at least seven, at least eight, at least nine, or at least ten mutations relative to the wildtype cytokine.

The term “cytokine antagonist” or “cytokine mask” refers to a moiety (e.g., a polypeptide) that binds to a cytokine and thereby inhibiting the cytokine from binding to its receptor on the surface of a target cell and/or exerting its biological functions while being bound by the antagonist or mask. Examples of a cytokine antagonist or mask include, without limitations, a polypeptide derived from an extracellular domain of the cytokine's natural receptor that makes contact with the cytokine, or a scFv or Fab of an antibody which binds to the cytokine and inhibits the binding of the cytokine to its receptor.

The term “effective amount” or “therapeutically effective amount” refers to an amount of a compound or composition sufficient to treat a specified disorder, condition, or disease, such as ameliorate, palliate, lessen, and/or delay one or more of its symptoms. In reference to a disease such as cancer, an effective amount may be an amount sufficient to delay cancer development or progression (e.g., decrease tumor growth rate, and/or delay or prevent tumor angiogenesis, metastasis, or infiltration of cancer cells into peripheral organs), reduce the number of epithelioid cells, cause cancer regression (e.g., shrink or eradicate a tumor), and/or prevent or delay cancer occurrence or recurrence. An effective amount can be administered in one or more administrations.

The term “functional analog” refers to a molecule that has the same biological specificity (e.g., binding to the same ligand) and/or activity (e.g., activating or inhibiting a target cell) as a reference molecule.

The term “fused” or “fusion” in reference to two polypeptide sequences refers to the joining of the two polypeptide sequences through a backbone peptide bond. Two polypeptides may be fused directly or through a peptide linker that is one or more amino acids long. A fusion polypeptide may be made by recombinant technology from a coding sequence containing the respective coding sequences for the two fusion partners, with or without a coding sequence for a peptide linker in between. In some embodiments, fusion encompasses chemical conjugation.

The term “pharmaceutically acceptable excipient” when used to refer to an ingredient in a composition means that the excipient is suitable for administration to a treatment subject, including a human subject, without undue deleterious side effects to the subject and without affecting the biological activity of the active pharmaceutical ingredient (API).

The term “subject” refers to a mammal and includes, but is not limited to, a human, a pet (e.g., a canine or a feline), a farm animal (e.g., cattle or horse), a rodent, or a primate.

As used herein, “treatment” or “treating” is an approach for obtaining beneficial or desired clinical results. Beneficial or desired clinical results include, but are not limited to, one or more of the following: alleviating one or more symptoms resulting from a disease, diminishing the extent of a disease, ameliorating a disease state, stabilizing a disease (e.g., preventing or delaying the worsening or progression of the disease), preventing or delaying the spread (e.g., metastasis) of a disease, preventing or delaying the recurrence of a disease, providing partial or total remission of a disease, decreasing the dose of one or more other medications required to treat a disease, increasing the patient's quality of life, and/or prolonging survival. The methods of the present disclosure contemplate any one or more of these aspects of treatment.

It is to be understood that one, some or all of the properties of the various embodiments described herein may be combined to form other embodiments of the present invention. The section headings used herein are for organizational purposes only and are not to be construed as limiting the subject matter described thereunder.

### IL-21 Prodrugs

The present disclosure provides IL-21 prodrugs that are metabolized in vivo to become active cytokine therapeutics. The IL-21 prodrugs have fewer side effects, better in vivo PK profiles (e.g., longer half-life) and better target specificity, and are more efficacious as compared to prior IL-21 therapeutics. In addition, when the masking moiety and the cytokine are fused to the different polypeptide chains, the interaction between the masking moiety and the cytokine provides the additional driving force to form the correct heterodimerization. This would make the manufacturing of the prodrug more efficient.

The present IL-21 prodrugs comprise an IL-21 agonist polypeptide (cytokine moiety) linked to a carrier moiety and masked (bound) by an IL-21 antagonist (masking moiety or cytokine mask). The IL-21 antagonist, which may be, for example, an antigen-binding fragment of antibody which binds human IL-21 or its analog, is linked to the cytokine moiety or to the carrier moiety through a cleavable linker (e.g., a cleavable peptide linker). The mask inhibits the cytokine moiety's biological functions while the mask is binding to it. The prodrugs may be activated at a target site (e.g., at a tumor site or the surrounding environment) in the patient by cleavage of the linker and the consequent release of the cytokine mask from the prodrug, exposing the previously masked cytokine moiety and allowing the cytokine moiety to bind to its receptor on a target cell and exert its biological functions on the target cell. In some embodiments, the carriers for the IL-21 prodrugs are antigen-binding moieties, such as antibodies, that bind an antigen at the target site.

In some embodiments, the present IL-21 prodrugs are metabolized to become active at a target site in the body targeted by the carrier. In further embodiments, the carrier in the prodrug is an antibody targeting a tumor antigen such that the IL-21 prodrug is delivered to a tumor site in a patient and is metabolized locally (e.g., inside or in the vicinity of the tumor microenvironment) through cleavage of the linker linking the cytokine mask to the carrier or the cytokine moiety, making the cytokine moiety available to interact with its receptor on a target cell and stimulating the target immune cells locally.

A. Cytokine Moieties of the Prodrugs

An IL-21 prodrug may comprise an IL-21 agonist polypeptide or “IL-21 polypeptide” (cytokine moiety), a carrier (carrier moiety), and an IL-21 antagonist (masking moiety), wherein the IL-21 agonist polypeptide is fused to the carrier directly or through a linker (e.g., cleavable or noncleavable peptide linker), and the IL-21 antagonist is linked to the IL-21 agonist polypeptide or to the carrier through a cleavable peptide linker. In the present IL-21 prodrugs, the IL-21 agonist polypeptide may be a wildtype IL-21 polypeptide such as a wildtype human IL-21 (SEQ ID NO: 1), or an IL-21 mutein such as one derived from a human IL-21, e.g., one with an amino acid sequence selected from SEQ ID NOs: 2-5. The IL-21 mutein may have significantly reduced affinity for IL-21Rα or IL-21RαRγ, as compared to wild type IL-21. In some embodiments, the IL-21 mutein has binding affinity for the high-affinity IL-2Rα that is 5 times, 10 times, 20 times, 50 times, 100 times, 300 times, 500 times, 1,000 times, or 10,000 times lower compared to wild type IL-21. Unless otherwise indicated, all residue numbers in IL-21 and IL-21 muteins described herein are in accordance with the numbering in SEQ ID NO: 1.

B. Masking Moieties of the IL-21 Prodrugs

The IL-21 antagonist, i.e., the masking moiety, in the present prodrug may comprise a peptide or an antibody or antibody fragment that binds to the cytokine moiety in the prodrug, masking the cytokine moiety and inhibiting its biological functions. In some embodiments, the IL-21 antagonist comprises a peptide identified from the screening of a peptide library. In some embodiments, the IL-21 antagonist comprises an antibody or antigen-binding fragment thereof that blocks the binding of IL-21 or IL-21 muteins to IL-21Rα and/or IL-21Rγ. In some embodiments, the antibody fragment in the prodrug is an scFv or Fab comprising heavy chain CDR1-3 and light chain CDR1-3 of an anti-IL-21 antibody selected from 19E3, 9F11, 8B6, or 9H10 disclosed in US Patent Publication No. US2020/0164069, the disclosure of which is incorporated herein in its entirety.

By way of example, IL-21 antagonists may comprise peptides and antibodies that bind IL-21 and interfere with the binding of the IL-21 to its receptors, leading to the reduced biological activities of the IL-21 moiety while masked. In some embodiments, the masking moiety comprises a binding fragment of an antibody which binds to the IL-21 agonist polypeptide; and wherein the antibody inhibits the binding of the IL-21 agonist polypeptide to IL-21Rα and/or IL-21Rγ.

In some embodiments, the antigen-binding moiety is the binding fragment of an antibody against human IL-21, wherein the antibody comprises a heavy chain variable domain with an amino acid sequence at least 95% identical as that of SEQ ID NO:97 or 99, and a light chain variable domain with an amino acid sequence at least 95% identical as that of SEQ ID NO:98 or 100. In particular embodiments, the antibody fragment is a single chain fragment variable (scFv) comprising a heavy chain variable domain with an amino acid sequence as shown SEQ ID NO:97 and a light chain variable domain with an amino acid sequence as shown in SEQ ID NO: 98, or a heavy chain variable domain with an amino acid sequence as shown SEQ ID NO:99 and a light chain variable domain with an amino acid sequence as shown in SEQ ID NO: 100.

In some embodiments, the masking moiety comprises an antigen-binding moiety, wherein the antigen-binding moiety comprises antibody avizakimab or a binding fragment thereof. Avizakimab (BOS161721) is a monoclonal antibody that inhibits interleukin-21 (IL-21) bioactivity (see, e.g., US20170173149. In some embodiments, the masking moiety is a Fab or scFv comprising the same light chain and heavy chain CDRs as derived from avizakimab. In some embodiment, the masking moiety comprises a scFv or Fab comprising the light chain and heavy chain variable domains of the antibody Ab327 described in US20150266954. In some embodiments, the masking moiety comprises scFv or Fab comprising the light chain and heavy chain variable domains of the antibodies (e.g., 19E3, 9F11, 8B6, or 9H10) described in US20200164069.

In some embodiments, the prodrug further comprises a second masking moiety. In some embodiments, the second masking moiety comprises an extracellular domain of IL-21R or functional analog thereof. In some embodiments, the extracellular domain of IL-21R is a mutated version of the extracellular domain (ECD) of human IL-21 receptor alpha (IL-21Rα ECD) with mutation or mutations at position or positions selected from H49, D122, P147, W148, A149, and V150 (numbering according to SEQ ID NO: 128). In some embodiments, the mutation or mutation is selected from: 1) H49N; 2) a mutation at position D122 selected from D122A, D122I, D122W, D122F, and D122Y; 3) a mutation at position P147 selected from P147G and P147N; 4) a mutation at position W148 selected from W148G, W148N, and W148S; 5) a mutation at position A149 selected from A149G and A149S; and 6) V150S. In some embodiments, the extracellular domain of IL-21R comprises a mutation or mutations which interrupt the interaction between IL-21R and IL-21Rγ.

C. Carrier Moieties of the Prodrugs

The carrier moieties of the present IL-21 prodrugs may be an antigen-binding moiety, or a moiety that is not an antigen-binding moiety. The carrier moiety may improve the PK profiles such as serum half-life of the cytokine agonist polypeptide, and may also target the cytokine agonist polypeptide to a target site in the body, such as a tumor site.

1. Antigen-Binding Carrier Moieties

The carrier moiety may be an antibody or an antigen-binding fragment thereof, or an immunoadhesin. In some embodiments, the antigen-binding moiety is a full-length antibody with two heavy chains and two light chains, a Fab fragment, a Fab′ fragment, a F(ab′)₂ fragment, a Fv fragment, a disulfide linked Fv fragment, a single domain antibody, a nanobody, or a single-chain variable fragment (scFv). In some embodiments, the antigen-binding moiety is a bispecific antigen-binding moiety and can bind to two different antigens or two different epitopes on the same antigen. The antigen-binding moiety may provide additional and potentially synergetic therapeutic efficacy to the cytokine agonist polypeptide.

The IL-21 agonist polypeptide and its mask may be fused to the N-terminus or C-terminus of the light chains and/or heavy chains of the antigen-binding moiety. By way of example, the IL-21 agonist polypeptide and its mask may be fused to the antibody heavy chain or an antigen-binding fragment thereof or to the antibody light chain or an antigen-binding fragment thereof. In some embodiments, the IL-21 agonist polypeptide is fused to the C-terminus of one or both of the heavy chains of an antibody, and the IL-21 mask is fused to the other terminus of the IL-21 agonist polypeptide through a cleavable peptide linker. In some embodiments, the IL-21 agonist polypeptide is fused to the C-terminus of one of the heavy chains of an antibody, and the IL-21 mask is fused to the C-terminus of the other heavy chain of the antibody through a cleavable peptide linker, wherein the two heavy chains contain mutations that allow the specific pairing of the two different heavy chains.

Strategies of forming heterodimers are well known (see, e.g., Spies et al., Mol Imm. (2015) 67(2)(A):95-106). For example, the two heavy chain polypeptides in the prodrug may form stable heterodimers through “knobs-into-holes” mutations. “Knobs-into-holes” mutations are made to promote the formation of the heterodimers of the antibody heavy chains and are commonly used to make bispecific antibodies (see, e.g., U.S. Pat. No. 8,642,745). For example, the Fc domain of the antibody may comprise a T366W mutation in the CH3 domain of the “knob chain” and T366S, L368A, and/or Y407V mutations in the CH3 domain of the “hole chain.” An additional interchain disulfide bridge between the CH3 domains can also be used, e.g., by introducing a Y349C mutation into the CH3 domain of the “knobs chain” and an E356C or S354C mutation into the CH3 domain of the “hole chain” (see, e.g., Merchant et al., Nature Biotech 16:677-81 (1998)). In other embodiments, the antibody moiety may comprise Y349C and/or T366W mutations in one of the two CH3 domains, and E356C, T366S, L368A, and/or Y407V mutations in the other CH3 domain. In certain embodiments, the antibody moiety may comprise Y349C and/or T366W mutations in one of the two CH3 domains, and S354C (or E356C), T366S, L368A, and/or Y407V mutations in the other CH3 domain, with the additional Y349C mutation in one CH3 domain and the additional E356C or S354C mutation in the other CH3 domain, forming an interchain disulfide bridge (numbering always according to EU index of Kabat; Kabat et al., “Sequences of Proteins of Immunological Interest,” 5th ed., Public Health Service, National Institutes of Health, Bethesda, Md. (1991)). Other knobs-into-holes technologies, such as those described in EP1870459A1, can be used alternatively or additionally. Thus, another example of knobs-into-holes mutations for an antibody moiety is having R409D/K370E mutations in the CH3 domain of the “knob chain” and D399K/E357K mutations in the CH3 domain of the “hole chain” (EU numbering).

In some embodiments, the antibody moiety in the prodrug comprises L234A and L235A (“LALA”) mutations in its Fc domain. The LALA mutations eliminate complement binding and fixation as well as Fcγ dependent ADCC (see, e.g., Hezareh et al. J. Virol. (2001) 75(24):12161-8). In further embodiments, the LALA mutations are present in the antibody moiety in addition to the knobs-into-holes mutations.

In some embodiments, the antibody moiety comprises the M252Y/S254T/T256E (“YTE”) mutations in the Fc domain. The YTE mutations allow the simultaneous modulation of serum half-life, tissue distribution and activity of IgG₁ (see Dall'Acqua et al., J Biol Chem. (2006) 281:23514-24; and Robbie et al., Antimicrob Agents Chemother. (2013) 57(12):6147-53). In further embodiments, the YTE mutations are present in the antibody moiety in addition to the knobs-into-holes mutations. In particular embodiments, the antibody moiety has YTE, LALA and knobs-into-holes mutations or any combination thereof.

The antigen-binding moiety may bind to an antigen on the surface of a cell, such as an immune cell, for example, T cells, NK cells, and macrophages, or bind to a cytokine. For example, the antigen-binding moiety may bind to PD-1, LAG-3, TIM-3, TIGIT, CTLA-4, or TGF-beta and may be an antibody. The antibody may have the ability to activate the immune cell and enhance its anti-cancer activity.

The antigen-binding moiety may bind to an antigen on the surface of a tumor cell. For example, the antigen-binding moiety may bind to FAP alpha, 5T4, Trop-2, PD-L1, HER-2, EGFR, Claudin 18.2, DLL-3, GCP3, or carcinoembryonic antigen (CEA), and may be an antibody. The antibody may or may not have ADCC activity. The antibody may also be further conjugated to a cytotoxic drug.

In some embodiments, the antigen-binding moiety binds to guanyl cyclase C (GCC), carbohydrate antigen 19-9 (CA19-9), glycoprotein A33 (gpA33), mucin 1 (MUC1), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostates-specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, CD47, signal-regulatory protein alpha (SIRPα), Claudin 18.2, Claudin 6, BCMA, or EPCAM. In some embodiments, the antigen-binding moiety binds to an epidermal growth factor (EGF)-like domain of DLL3. In some embodiments, the antigen-binding moiety binds to a Delta/Serrate/Lag2 (DSL)-like domain of DLL3. In some embodiments, the antigen-binding moiety binds to an epitope located after the 374th amino acid of GPC3. In some embodiments, the antigen-binding moiety binds to a heparin sulfate glycan of GPC3. In some embodiments, the antigen-binding moiety binds to Claudin 18.2 and does not bind to Claudin 18.1. In some embodiments, the antigen-binding moiety binds to Claudin 18.1 with at least 10 times weaker binding affinity than to Claudin 18.2.

Exemplary antigen-binding moieties include trastuzumab, rituximab, brentuximab, cetuximab, panitumumab, GC33 (or a humanized version thereof), anti-EGFR antibody mAb806 (or a humanized version thereof), anti-dPNAG antibody F598, and antigen-binding fragments thereof. In some embodiments, the antigen-binding moiety has at least 90%, 91%, 92%, 93%, 94%, 95%, 96%, 97%, 98%, or 99% identity to trastuzumab, rituximab, brentuximab, cetuximab, or panitumumab, GC33 (or a humanized version thereof), anti-EGFR antibody mAb806 (or a humanized version thereof), anti-dPNAG antibody F598, or a fragment thereof. In some embodiments, the antigen-binding moiety has an antibody heavy chain with at least 90%, 91%, 92%, 93%, 94%, 95%, 96%, 97%, 98%, or 99% identity to the antibody heavy chain of trastuzumab, rituximab, brentuximab, cetuximab, panitumumab, GC33 (or a humanized version thereof), anti-EGFR antibody mAb806 (or a humanized version thereof), anti-dPNAG antibody F598, or a fragment thereof. In some embodiments, the antigen-binding moiety has an antibody light chain with at least 90%, 91%, 92%, 93%, 94%, 95%, 96%, 97%, 98%, or 99% identity to the antibody light chain of trastuzumab, rituximab, brentuximab, cetuximab, panitumumab, GC33 (or a humanized version thereof), anti-EGFR antibody mAb806 (or a humanized version thereof), anti-dPNAG antibody F598, or a fragment thereof. The antigen-binding moiety is fused to an IL-2 agonist polypeptide. In some embodiments, the antigen-binding moiety comprises the six complementarity determining regions (CDRs) of trastuzumab, rituximab, brentuximab, cetuximab, panitumumab, GC33, anti-EGFR antibody mAb806, or anti-dPNAG antibody F598.

A number of CDR delineations are known in the art and are encompassed herein. A person of skill in the art can readily determine a CDR for a given delineation based on the sequence of the heavy or light chain variable region. The “Kabat” CDRs are based on sequence variability and are the most commonly used (Kabat et al., Sequences of Proteins of Immunological Interest, 5th Ed. Public Health Service, National Institutes of Health, Bethesda, Md. (1991)). “Chothia” CDRs refer to the location of the structural loops (Chothia & Lesk, Canonical structures for the hypervariable regions of immunoglobulins, J Mol Biol. (1987) 196:901-17). The “AbM” CDRs represent a compromise between the Kabat CDRs and Chothia structural loops, and are used by Oxford Molecular's AbM antibody modeling software. The “Contact” CDRs are based on an analysis of the available complex crystal structures. The residues from each of these CDRs are noted below in Table 1, in reference to common antibody numbering schemes. Unless otherwise specified herein, amino acid numbers in antibodies refer to the Kabat numbering scheme as described in Kabat et al., supra, including when CDR delineations are made in reference to Kabat, Chothia, AbM, or Contact schemes. Using this numbering system, the actual linear amino acid sequence may contain fewer or additional amino acids corresponding to a shortening of, or insertion into, a framework region (FR) or CDR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

TABLE 1
CDR Delineations According to Various Schemes

| CDR | Kabat | AbM | Chothia | Contact |
|---|---|---|---|---|
| VL-CDR1 | L24-L34 | L24-L34 | L26-L32 | L30-L36 |
| VL-CDR2 | L50-L56 | L50-L56 | L50-L52 | L46-L55 |
| VL-CDR3 | L89-L97 | L89-L97 | L91-L96 | L89-L96 |
| VH-CDR1 (Kabat nos.) | H31-H35B | H26-H35B | H26-H32 | H30-H35B |
| VH-CDR1 (Chothia nos.) | H31-H35 | H26-H35 | H26-H32 | H30-H35 |
| VH-CDR2 | H50-H65 | H50-H58 | H53-H55 | H47-H58 |
| VH-CDR3 | H95-H102 | H95-H102 | H95-H101 | H93-H101 |

In some embodiments, the CDRs are “extended CDRs,” and encompass a region that begins or terminates according to a different scheme. For example, an extended CDR can be as follows: L24-L36, L26-L34, or L26-L36 (VL-CDR1); L46-L52, L46-L56, or L50-L55 (VL-CDR2); L91-L97 (VL-CDR3); H47-H55, H47-H65, H50-H55, H53-H58, or H53-H65 (VH-CDR2); and/or H93-H102 (VH-CDR3).

In some embodiments, the antigen-binding moiety binds to HER2, and comprises a light chain having an amino acid sequence with at least 90%, 91%, 92%, 93%, 94%, 95%, 96%, 97%, 98%, or 99% identity to SEQ ID NO: 52, or a fragment thereof, and a heavy chain having an amino acid sequence with at least 90%, 91%, 92%, 93%, 94%, 95%, 96%, 97%, 98%, or 99% identity to SEQ ID NO: 53, or a fragment thereof. In some embodiments, the antigen-binding domain comprises CDR1, CDR2, and CDR3 from SEQ ID NO: 52, and CDR1, CDR2, and CDR3 from SEQ ID NO: 53.

In some embodiments, the antigen-binding moiety binds to CD20, and comprises a light chain having an amino acid sequence with at least 90%, 91%, 92%, 93%, 94%, 95%, 96%, 97%, 98%, or 99% identity to SEQ ID NO: 54, or a fragment thereof, and a heavy chain having an amino acid sequence with at least 90%, 91%, 92%, 93%, 94%, 95%, 96%, 97%, 98%, or 99% identity to SEQ ID NO: 55, or a fragment thereof. In some embodiments, the antigen-binding domain comprises CDR1, CDR2, and CDR3 from SEQ ID NO: 54, and CDR1, CDR2, and CDR3 from SEQ ID NO: 55.

In some embodiments, the antigen-binding moiety binds to CD30, and comprises a light chain having an amino acid sequence with at least 90%, 91%, 92%, 93%, 94%, 95%, 96%, 97%, 98%, or 99% identity to SEQ ID NO: 56, or a fragment thereof, and a heavy chain having an amino acid sequence with at least 90%, 91%, 92%, 93%, 94%, 95%, 96%, 97%, 98%, or 99% identity to SEQ ID NO: 57, or a fragment thereof. In some embodiments, the antigen-binding domain comprises CDR1, CDR2, and CDR3 from SEQ ID NO: 56, and CDR1, CDR2, and CDR3 from SEQ ID NO: 57.

In some embodiments, the antigen-binding moiety binds to EGFR, and comprises a light chain having an amino acid sequence with at least 90%, 91%, 92%, 93%, 94%, 95%, 96%, 97%, 98%, or 99% identity to SEQ ID NO: 58 or 60, or a fragment thereof, and a heavy chain having an amino acid sequence with at least 90%, 91%, 92%, 93%, 94%, 95%, 96%, 97%, 98%, or 99% identity to SEQ ID NO: 59 or 61, or a fragment thereof. In some embodiments, the antigen-binding domain comprises CDR1, CDR2, and CDR3 from SEQ ID NO: 58 or 60, and CDR1, CDR2, and CDR3 from SEQ ID NO: 59 or 61.

In some embodiments, the antigen-binding moiety binds to c-MET, and comprises a light chain having an amino acid sequence with at least 90%, 91%, 92%, 93%, 94%, 95%, 96%, 97%, 98%, or 99% identity to SEQ ID NO: 62, or a fragment thereof, and a heavy chain having an amino acid sequence with at least 90%, 91%, 92%, 93%, 94%, 95%, 96%, 97%, 98%, or 99% identity to SEQ ID NO: 63, or a fragment thereof. In some embodiments, the antigen-binding domain comprises CDR1, CDR2, and CDR3 from SEQ ID NO: 62, and CDR1, CDR2, and CDR3 from SEQ ID NO: 63.

In some embodiments, the antigen-binding moiety binds to GPC3, and comprises a light chain having an amino acid sequence with at least 90%, 91%, 92%, 93%, 94%, 95%, 96%, 97%, 98%, or 99% identity to SEQ ID NO: 64, or a fragment thereof, and a heavy chain having an amino acid sequence with at least 90%, 91%, 92%, 93%, 94%, 95%, 96%, 97%, 98%, or 99% identity to SEQ ID NO: 65, or a fragment thereof. In some embodiments, the antigen-binding domain comprises CDR1, CDR2, and CDR3 from SEQ ID NO: 64, and CDR1, CDR2, and CDR3 from SEQ ID NO: 65.

In some embodiments, the antigen-binding moiety binds to Claudin 18.2, and comprises a light chain having an amino acid sequence with at least 90%, 91%, 92%, 93%, 94%, 95%, 96%, 97%, 98%, or 99% identity to SEQ ID NO: 66, or a fragment thereof, and a heavy chain having an amino acid sequence with at least 90%, 91%, 92%, 93%, 94%, 95%, 96%, 97%, 98%, or 99% identity to SEQ ID NO: 67, or a fragment thereof. In some embodiments, the antigen-binding domain comprises CDR1, CDR2, and CDR3 from SEQ ID NO: 66, and CDR1, CDR2, and CDR3 from SEQ ID NO: 67.

In some embodiments, the antigen-binding moiety binds to FAP alpha, and comprises a light chain having an amino acid sequence with at least 90%, 91%, 92%, 93%, 94%, 95%, 96%, 97%, 98%, or 99% identity to SEQ ID NO: 80 or 81, or a fragment thereof, and a heavy chain having an amino acid sequence with at least 90%, 91%, 92%, 93%, 94%, 95%, 96%, 97%, 98%, or 99% identity to SEQ ID NO: 82, or a fragment thereof. In some embodiments, the antigen-binding domain comprises CDR1, CDR2, and CDR3 from SEQ ID NO: 80 or 81, and CDR1, CDR2, and CDR3 from SEQ ID NO: 82.

In some embodiments, the antigen-binding moiety binds to FAP alpha, and comprises a light chain variable domain having an amino acid sequence with at least 90%, 91%, 92%, 93%, 94%, 95%, 96%, 97%, 98%, or 99% identity to SEQ ID NO: 83, and a heavy chain variable domain having an amino acid sequence with at least 90%, 91%, 92%, 93%, 94%, 95%, 96%, 97%, 98%, or 99% identity to SEQ ID NO: 84. In some embodiments, the antigen-binding domain comprises CDR1, CDR2, and CDR3 from SEQ ID NO: 84, and CDR1, CDR2, and CDR3 from SEQ ID NO: 84.

In some embodiments, the antigen-binding moiety binds to PDL1, and comprises a light chain having an amino acid sequence with at least 90%, 91%, 92%, 93%, 94%, 95%, 96%, 97%, 98%, or 99% identity to SEQ ID NO: 89, or a fragment thereof, and a heavy chain having an amino acid sequence with at least 90%, 91%, 92%, 93%, 94%, 95%, 96%, 97%, 98%, or 99% identity to SEQ ID NO: 90, or a fragment thereof. In some embodiments, the antigen-binding domain comprises CDR1, CDR2, and CDR3 from SEQ ID NO: 89, and CDR1, CDR2, and CDR3 from SEQ ID NO: 90.

In some embodiments, the antigen-binding moiety binds to 5T4, and comprises a light chain variable domain having an amino acid sequence with at least 90%, 91%, 92%, 93%, 94%, 95%, 96%, 97%, 98%, or 99% identity to SEQ ID NO: 87 or 88, and a heavy chain variable domain having an amino acid sequence with at least 90%, 91%, 92%, 93%, 94%, 95%, 96%, 97%, 98%, or 99% identity to SEQ ID NO: 85 or 86, or a fragment thereof. In some embodiments, the antigen-binding domain comprises CDR1, CDR2, and CDR3 from SEQ ID NO: 87 or 88, and CDR1, CDR2, and CDR3 from SEQ ID NO: 85 or 86.

In some embodiments, the antigen-binding moiety binds to Trop-2, and comprises a light chain variable region comprising a CDR1 comprising an amino acid sequence of KASQDVSIAVA (SEQ ID NO: 68), a CDR2 comprising an amino acid sequence of SASYRYT (SEQ ID NO: 69), and a CDR3 comprising an amino acid sequence of QQHYITPLT (SEQ ID NO: 70); and a heavy chain variable region comprising a CDR1 comprising an amino acid sequence of NYGMN (SEQ ID NO: 71), a CDR2 comprising an amino acid sequence of WINTYTGEPTYTDDFKG (SEQ ID NO: 72), and a CDR3 comprising an amino acid sequence of GGFGSSYWYFDV (SEQ ID NO: 73).

In some embodiments, the antigen-binding moiety binds to mesothelin, and comprises light chain variable region comprising a CDR1 comprising an amino acid sequence of SASSSVSYMH (SEQ ID NO: 74), a CDR2 comprising an amino acid sequence of DTSKLAS (SEQ ID NO: 75), and a CDR3 comprising an amino acid sequence of QQWSGYPLT (SEQ ID NO: 76); and a heavy chain variable region comprising a CDR1 comprising an amino acid sequence of GYTMN (SEQ ID NO:77), a CDR2 comprising an amino acid sequence of LITPYNGASSYNQKFRG (SEQ ID NO: 78), and a CDR3 comprising an amino acid sequence of GGYDGRGFDY (SEQ ID NO: 79).

In some embodiments, the antigen-binding moiety binds to PD-1, and comprises a light chain having an amino acid sequence with at least 90%, 91%, 92%, 93%, 94%, 95%, 96%, 97%, 98%, or 99% identity to SEQ ID NO: 50, or a fragment thereof, and a heavy chain having an amino acid sequence with at least 90%, 91%, 92%, 93%, 94%, 95%, 96%, 97%, 98%, or 99% identity to SEQ ID NO: 91, or a fragment thereof. In some embodiments, the antigen-binding domain comprises CDR1, CDR2, and CDR3 from SEQ ID NO: 50, and CDR1, CDR2, and CDR3 from SEQ ID NO: 91.

In some embodiments, the antigen-binding moiety binds to PD-1, and comprises a light chain having an amino acid sequence with at least 90%, 91%, 92%, 93%, 94%, 95%, 96%, 97%, 98%, or 99% identity to SEQ ID NO: 92, or a fragment thereof, and a heavy chain having an amino acid sequence with at least 90%, 91%, 92%, 93%, 94%, 95%, 96%, 97%, 98%, or 99% identity to SEQ ID NO: 93, or a fragment thereof. In some embodiments, the antigen-binding domain comprises CDR1, CDR2, and CDR3 from SEQ ID NO: 92, and CDR1, CDR2, and CDR3 from SEQ ID NO: 93.

In some embodiments, the antigen-binding moiety comprises one, two or three antigen-binding domains. For example, the antigen-binding moiety is bispecific and binds to two different antigens selected from the group consisting of HER2, HER3, EGFR, 5T4, FAP alpha, Trop-2, GPC3, VEGFR2, Claudin 18.2 and PD-L1. In some embodiments, the bispecific antigen-binding moiety binds to two different epitopes of HER2.

2. Other Carrier Moieties

Other non-antigen-binding carrier moieties may be used for the present prodrugs. For example, an antibody Fc domain (e.g., a human IgG₁, IgG₂, IgG₃, or IgG₄ Fc), a polymer (e.g., PEG), an albumin (e.g., a human albumin) or a fragment thereof, or a nanoparticle can be used. By way of example, the IL-21 agonist polypeptide and its antagonist may be fused to an antibody Fc domain, forming an Fc fusion protein. In some embodiments, the IL-21 agonist polypeptide is fused (directly or through a peptide linker) to the C-terminus or N-terminus of one of the Fc domain polypeptide chains, and the cytokine mask is fused to the corresponding C-terminus or N-terminus of the other Fc domain polypeptide chain through a cleavable peptide linker, wherein the two Fc domain polypeptide chains contain mutations that allow the specific pairing of the two different Fc chains. In some embodiments, the Fc domain comprises the holes-into-holes mutations described above. In further embodiments, the Fc domain may comprise also the YTE and/or LALA mutations described above.

D. Linker Components of the Prodrugs

The IL-21 agonist polypeptide may be fused to the carrier moiety with or without a peptide linker. The peptide linker may be non-cleavable. In some embodiments, the peptide linker is selected from SEQ ID NOs:29-33 and 132. In particular embodiments, the peptide linker comprise the amino acid sequence GGGGSGGGGSGGGGS (SEQ ID NO: 31).

The IL-21 mask may be fused to the cytokine moiety or to the carrier through a cleavable peptide linker. The cleavable linker may contain one or more (e.g., two or three) cleavable moieties (CM). Each CM may be a substrate for an enzyme or protease selected from legumain, plasmin, TMPRSS-3/4, MMP-2, MMP-9, MT1-MMP, cathepsin, caspase, human neutrophil elastase, beta-secretase, uPA, and PSA. Examples of cleavable linkers include, without limitation, those comprising an amino acid sequence selected from SEQ ID NOs: 17-26. By way of example, a cleavable peptide linker is used to link the masking moiety to the carrier or to the cytokine moiety.

In some aspect, this disclosure also presents prodrugs without cleavable peptide linkers. In some embodiments, a prodrug of the present disclosure comprises a cytokine moiety, a masking moiety, and a carrier moiety, wherein:
a. the masking moiety binds to the cytokine moiety and inhibits an intended biological activity of the cytokine moiety;
b. the carrier moiety comprises an antigen binding moiety;
c. the masking moiety is linked indirectly to the carrier moiety through a non-cleavable peptide linker or directly without a peptide linker; and where
d. the cytokine moiety has lower intended biological activity compared to the cytokine moiety of an activated fusion molecule that comprises the same carrier moiety and the same cytokine moiety but without the masking moiety.

In some embodiments, the IL-21 masking moiety of the present disclosure may be fused to the cytokine moiety or to the carrier through a non-cleavable peptide linker. In some embodiments, the peptide linker is selected from SEQ ID NOs:29-33 and 132. In particular embodiments, the peptide linker comprise the amino acid sequence GGGGSGGGGSGGGGS (SEQ ID NO: 31) or GGGGSGGGGSAAGGGGSGGGGS (SEQ ID NO: 132).

E. IL-21 Prodrugs with an Additional Effector Polypeptide

In specific embodiments, the IL-21 prodrugs further comprise a second effector polypeptide such as a second cytokine moiety. In such cases, the prodrugs may further comprise a second masking moiety that binds to and inhibits a biological activity of the second effector polypeptide.

By way of example, the IL-21 agonist polypeptide and its mask may be fused to separate Fc chains at one end of the Fc domain, while the second cytokine moiety and its mask may be fused to separate Fc chains at the other end of the Fc domain, wherein the masks are fused to the Fc chains through cleavable peptide linkers. In certain embodiments, the two Fc domain polypeptide chains contain mutations that allow the specific pairing of the two different Fc chains.

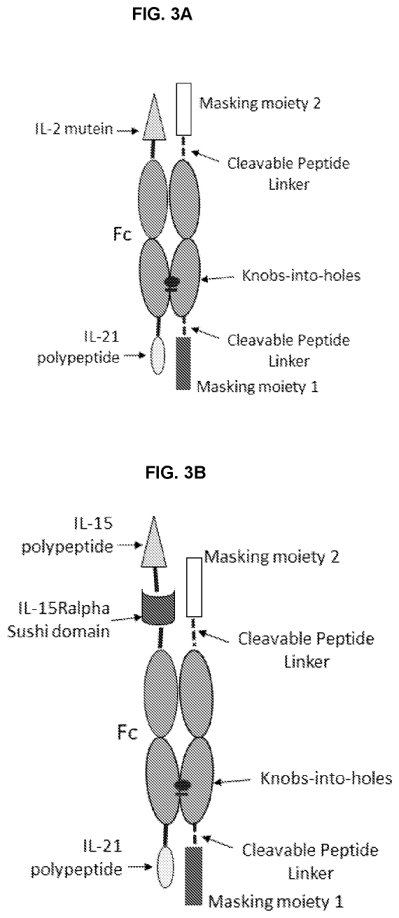

Examples of prodrugs comprising two effector polypeptides and two masking moieties include those comprising two polypeptide chains whose amino acid sequences respectively comprise (i) SEQ ID NOs: 42 and 113; (ii) SEQ ID NOs: 42 and 114; (iii) SEQ ID NOs: 42 and 115; (iv) SEQ ID NOs: 42 and 116; (v) SEQ ID NOs: 43 and 113; (vi) SEQ ID NOs: 43 and 114; (vii) SEQ ID NOs: 43 and 115; or (viii) SEQ ID NOs: 43 and 116. The exemplary structure of an IL-21 prodrug that comprises an IL-2 agonist polypeptide (second effector polypeptide) and its corresponding mask is illustrated in FIG. 3A. The exemplary structure of an IL-21 prodrug that comprises an IL-15 agonist polypeptide, the sushi domain and its corresponding mask is illustrated in FIG. 3B.

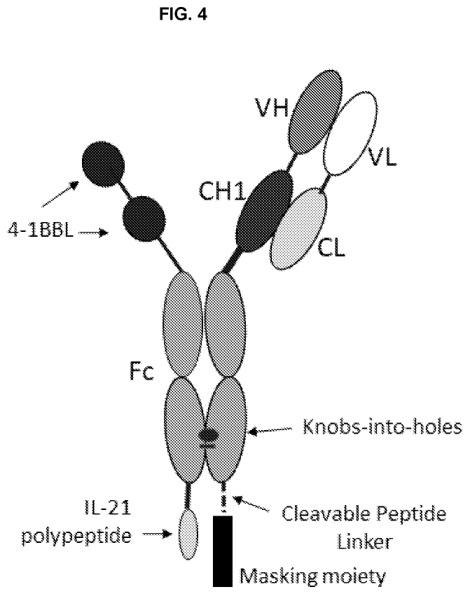

In some embodiments, the IL-21 prodrugs further comprise two or three copies of the ectodomains of the ligand of a tumor necrosis factor (TNF) superfamily member. In some embodiments, the TNF superfamily member is 4-1BB. The structure of an exemplary IL-21 prodrug comprising two copies of a 4-1BB ligand (4-1BBL) ectodomain is illustrated in FIG. 4. The carrier of the IL-21 prodrug may be an antibody that binds to an antigen expressed in a tumor, for example, FAP or 5T4.

Specific, nonlimiting examples of IL-21 agonist polypeptides, cytokine masks, carriers, peptide linkers, and prodrugs are shown in the Sequences section below. Further, the prodrugs of the present disclosure may be made by well known recombinant technology. For examples, one or more expression vectors comprising the coding sequences for the polypeptide chains of the prodrugs may be transfected into mammalian host cells (e.g., CHO cells), and the cells are cultured under conditions that allow the expression of the coding sequences and the assembly of the expressed polypeptides into the prodrug complex. In order for the prodrug to remain inactive, the host cells that express no or little uPA, matriptase, MMP-2 and/or MMP-9 may be used. In some embodiments, the host cells may contain null mutations (knockout) of the genes that encode these proteases.

### Pharmaceutical Compositions

Pharmaceutical compositions comprising the prodrugs and muteins (i.e., the active pharmaceutical ingredient or API) of the present disclosure may be prepared by mixing the API having the desired degree of purity with one or more optional pharmaceutically acceptable excipients (see, e.g., Remington's Pharmaceutical Sciences, 16th Edition., Osol, A. Ed. (1980)) in the form of lyophilized formulations or aqueous solutions. Pharmaceutically acceptable excipients (or carriers) are generally nontoxic to recipients at the dosages and concentrations employed, and include, but are not limited to: buffers containing, for example, phosphate, citrate, succinate, histidine, acetate, or another inorganic or organic acid or salt thereof; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including sucrose, glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof, such as citrate, phosphate, succinate, tartrate, fumarate, gluconate, oxalate, lactate, and acetate. Additionally, buffers may comprise histidine and trimethylamine salts such as Tris.

Preservatives are added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents, sometimes known as “stabilizers” are present to adjust or maintain the tonicity of liquid in a composition. When used with large, charged biomolecules such as proteins and antibodies, they are often termed “stabilizers” because they can interact with the charged groups of the amino acid side chains, thereby lessening the potential for inter- and intra-molecular interactions. Tonicity agents can be present in any amount between 0.1% to 25% by weight, or more preferably between 1% to 5% by weight, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

Non-ionic surfactants or detergents (also known as “wetting agents”) are present to help solubilize the therapeutic agent as well as to protect the therapeutic protein against agitation-induced aggregation, which also permits the formulation to be exposed to shear surface stress without causing denaturation of the active therapeutic protein or antibody. Non-ionic surfactants are present in a range of about 0.05 mg/ml to about 1.0 mg/ml, preferably about 0.07 mg/ml to about 0.2 mg/ml.

Suitable non-ionic surfactants include polysorbates (20, 40, 60, 65, 80, etc.), polyoxamers (184, 188, etc.), PLURONIC® polyols, TRITON®, polyoxyethylene sorbitan monoethers (TWEEN®-20, TWEEN®-80, etc.), lauromacrogol 400, polyoxyl 40 stearate, polyoxyethylene hydrogenated castor oil 10, 50 and 60, glycerol monostearate, sucrose fatty acid ester, methyl cellulose and carboxymethyl cellulose. Anionic detergents that can be used include sodium lauryl sulfate, dioctyle sodium sulfosuccinate and dioctyl sodium sulfonate. Cationic detergents include benzalkonium chloride or benzethonium chloride.

The choice of pharmaceutical carrier, excipient or diluent may be selected with regard to the intended route of administration and standard pharmaceutical practice. Pharmaceutical compositions may additionally comprise any suitable binder(s), lubricant(s), suspending agent(s), coating agent(s) or solubilizing agent(s).

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route.

In some embodiments, the pharmaceutical composition of the present disclosure is a lyophilized protein formulation. In other embodiments, the pharmaceutical composition may be an aqueous liquid formulation.

### Methods of Treatment

The IL-21 prodrug can be used to treat a disease, depending on the antigen bound by the antigen-binding domain. In some embodiments, the IL-21 prodrug is used to treat cancer. In some embodiments, the IL-21 prodrug is used to treat an infection.

In some embodiments, a method of treating a disease (such as cancer, a viral infection, or a bacterial infection) in a subject comprises administering to the subject an effective amount of an IL-21 prodrug.

In some embodiments, the cancer is a solid cancer. In some embodiments, the cancer is a blood cancer or a solid tumor. Exemplary cancers that may be treated include, but are not limited to, leukemia, lymphoma, kidney cancer, bladder cancer, urinary tract cancer, cervical cancer, brain cancer, head and neck cancer, skin cancer, uterine cancer, testicular cancer, esophageal cancer, liver cancer, colorectal cancer, stomach cancer, squamous cell carcinoma, prostate cancer, pancreatic cancer, lung cancer such as non-small cell lung cancer, cholangiocarcinoma, breast cancer, and ovarian cancer.

In some embodiments, the IL-21 prodrug is used to treat a viral infection. In some embodiments, the virus causing the viral infection is hepatitis C (HCV), hepatitis B (HBV), human immunodeficiency virus (HIV), or a human papilloma virus (HPV). In some embodiments, the antigen-binding moiety binds to a viral antigen.

In some embodiments, the IL-21 prodrug is used to treat a bacterial infection such as sepsis. In some embodiments, the bacteria causing the bacterial infection is a drug-resistant bacteria. In some embodiments, the antigen-binding moiety binds to a bacterial antigen.

Generally, dosages and routes of administration of the present pharmaceutical compositions are determined according to the size and conditions of the subject, according to standard pharmaceutical practice. In some embodiments, the pharmaceutical composition is administered to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, intracranially, or intraspinally. In some embodiments, the composition is administered to a subject intravenously.

In some embodiments, the dosage of the pharmaceutical composition is a single dose or a repeated dose. In some embodiments, the doses are given to a subject once per day, twice per day, three times per day, or four or more times per day. In some embodiments, about 1 or more (such as about 2, 3, 4, 5, 6, or 7 or more) doses are given in a week. In some embodiments, the pharmaceutical composition is administered weekly, once every 2 weeks, once every 3 weeks, once every 4 weeks, weekly for two weeks out of 3 weeks, or weekly for 3 weeks out of 4 weeks. In some embodiments, multiple doses are given over the course of days, weeks, months, or years. In some embodiments, a course of treatment is about 1 or more doses (such as about 2, 3, 4, 5, 7, 10, 15, or 20 or more doses).

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It is understood that aspects and variations of the invention described herein include “consisting” and/or “consisting essentially of” aspects and variations. All publications and other references mentioned herein are incorporated by reference in their entirety. Although a number of documents are cited herein, this citation does not constitute an admission that any of these documents forms part of the common general knowledge in the art.

### Exemplary Embodiments

Further particular embodiments of the present disclosure are described as follows. These embodiments are intended to illustrate the compositions and methods described in the present disclosure and are not intended to limit the scope of the present disclosure.

1. A prodrug comprising a human IL-21 polypeptide, a masking moiety, and a carrier moiety,

wherein
the masking moiety binds to the human IL-21 polypeptide and inhibits a biological activity of the human IL-21 polypeptide,
the human IL-21 polypeptide is fused to the carrier moiety,
the masking moiety is fused to the human IL-21 polypeptide or to the carrier moiety through a cleavable or non-cleavable peptide linker, and
the masking moiety comprises a mutated version of the extracellular domain (ECD) of human IL-21 receptor alpha (IL-21Rα ECD) with mutation or mutations at position or positions selected from H49, S112, G113, Q114, D122, P147, W148, A149, V150, R153, K155, L156, S158, D160, S161, R162, S163, S165, and P168 (numbering according to SEQ ID NO: 6).
   2. The prodrug of embodiment 1, wherein the human IL-21 polypeptide comprises SEQ ID NO: 1, 2, 3, 4, or 5, or an amino acid sequence that is at least 90% identical to SEQ ID NO: 1, 2, 3, 4, or 5.
   3. The prodrug of embodiment 2, wherein the human IL-21 polypeptide comprises one or more mutations at positions selected from D18, Q19, E109, and K117 (numbering according to SEQ ID NO: 1).
   4. The prodrug of any one of embodiments 1-3, herein the masking moiety comprises a mutated version of IL-21Rα ECD, wherein said mutated IL-21Rα ECD comprises mutation or mutations selected from:
H49N,
a mutation at position D122 selected from D122A, D122I, D122W, D122F, and D122Y,
a mutation at position P147 selected from P147G and P147N,
a mutation at position W148 selected from W148G, W148N, and W148S,
a mutation at position A149 selected from A149G and A149S, and
a mutation V150S.
   5. The prodrug of any of embodiment 4, wherein said mutated IL-21Rα ECD further comprises mutation or mutations at a site or sites selected from S112, G113, Q114, R153, S158, K155, L156, D160, S161, R162, S163, S165, and P168.
   6. The prodrug of any of embodiments 1-5, wherein said IL-21Rα ECD comprises mutations P147G, W148S, and A149G; and wherein said IL-21Rα ECD mutein further comprises one or more mutations selected from the following:
a. S112G or S112A;
b. Q114E or Q114D;
c. R153E or R153D;
d. K155E or K155D;
e. L156T or L156A;
f. S168G or S158A;
g. D160G or D160K;
h. S161G;
i. S163G or S163A;
j. S165G or S163A; and
k. P168A or P168S.
   7. The prodrug of any of embodiments 1-6, wherein said IL-21Rα ECD mutein comprises an amino acid sequence selected from SEQ ID NOs: 98-108, or an amino acid sequence that is at least 90% identical to one selected from SEQ ID NOs: 98-108.
   8. The prodrug of any one of embodiments 1-7, further comprising a second cytokine moiety.
   9. The prodrug of embodiment 8, wherein the second cytokine moiety is
(i) a human IL-2 agonist polypeptide comprising SEQ ID NO: 8 or an amino acid sequence that is at least 90% identical to SEQ ID NO: 8,
(ii) a human IL-7 agonist polypeptide,
(iii) a human IL-9 agonist polypeptide,
(iv) a human IL-15 agonist polypeptide comprising SEQ ID NO: 9 or an amino acid sequence that is at least 90% identical to SEQ ID NO: 9,
(v) a human IL-15 agonist polypeptide and a human IL-15 receptor alpha sushi domain, or
(vi) a human CCL19 polypeptide comprising SEQ ID NO: 27 or an amino acid sequence that is at least 90% identical to SEQ ID NO: 27.
   10. The prodrug of embodiment 8 or 9, further comprising a second masking moiety that binds to the second cytokine moiety and inhibits a biological activity of the second cytokine moiety, wherein the second masking moiety is fused to the second cytokine moiety or to the carrier moiety through a cleavable peptide linker.
   11. The prodrug of embodiment 10, wherein the second masking moiety is selected from an ECD of IL-2 receptor beta subunit or a functional analog thereof, an ECD of an IL-7 receptor or a functional analog thereof, and an ECD of an IL-9 receptor or a functional analog thereof.
   12. The prodrug of any one of the preceding embodiments, wherein the human IL-21 polypeptide and/or the second cytokine moiety is fused to the carrier moiety through a noncleavable peptide linker.
   13. The prodrug of embodiment 12, wherein the noncleavable peptide linker comprises an amino acid sequence selected from SEQ ID NOs: 29-33.
   14. The prodrug of any one of the preceding embodiments, wherein the cleavable peptide linker comprises a substrate sequence of urokinase-type plasminogen activator (uPA), matrix metallopeptidase (MMP) 2, or MMP9.
   15. The prodrug of embodiment 14, wherein the cleavable peptide linker comprises substrate sequences of (i) both uPA and MMP2, (ii) both uPA and MMP9, or (iii) uPA, MMP2 and MMP9.
   16. The prodrug of embodiment 14, wherein the cleavable peptide linker comprises an amino acid sequence selected from SEQ ID NOs: 11-26.
   17. The prodrug of any one of the preceding embodiments, wherein the cleavable peptide linker is cleavable by one or more proteases located at a tumor site or its surrounding environment, and the cleavage leads to activation of the prodrug at the tumor site or surrounding environment.
   18. The prodrug of any one of the preceding embodiments, wherein the carrier moiety is a PEG molecule, an albumin, an albumin fragment, an antibody Fc domain, or an antibody or an antigen-binding fragment thereof.
   19. The prodrug of embodiment 18, wherein the carrier moiety is an antibody Fc domain or an antibody that comprises L234A and L235A (“LALA”) mutations (EU numbering).
   20. The prodrug of embodiment 18 or 19, wherein the carrier moiety is an antibody Fc domain or an antibody comprising knobs-into-holes mutations, and wherein
the human IL-21 polypeptide and its masking moiety are fused to different polypeptide chains of the antibody Fc domain or to the different heavy chains of the antibody, and optionally
the second cytokine moiety and the second masking moiety also are fused to different polypeptide chains of the antibody Fc domain or to the different heavy chains of the antibody.
   21. The prodrug of embodiment 20, wherein the human IL-21 polypeptide and its masking moiety are fused to the C-termini of the two different polypeptide chains of the Fc domain or to the C-termini of the two different heavy chains of the antibody.
   22. The prodrug of embodiment 20, wherein the human IL-21 polypeptide and its masking moiety are fused to the N-termini of the two different polypeptide chains of the Fc domain or to the N-termini of the two different heavy chains of the antibody.

The prodrug of embodiments 21 and 22, wherein the second cytokine moiety and the second masking moiety are fused to the opposite termini of the two different polypeptide chains of the Fc domain, or to the opposite termini of the two different heavy chains of the antibody, from the human IL-21 polypeptide and its masking moiety.

23. The prodrug of any one of embodiments 20-23, wherein the knobs-into-holes mutations comprise a T366Y “knob” mutation on a polypeptide chain of the Fc domain or a heavy chain of the antibody, and a Y407T “hole” mutation in the other polypeptide of the Fc domain or the other heavy chain of the antibody (EU numbering).
24. The prodrug of any one of embodiments 20-23, wherein the knobs-into-holes mutations comprise Y349C and/or T366W mutations in the CH3 domain of the “knob chain” and E356C, T366S, L368A, and/or Y407V mutations in the CH3 domain of the “hole chain” (EU numbering).
25. The prodrug of any one of embodiments 18-25, wherein the carrier moiety is an antibody or an antigen-binding fragment thereof that specifically binds to one or more antigens selected from Guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, CD47, signal-regulatory protein alpha (SIRPα), PD1, Claudin 18.2, Claudin 6, 5T4, BCMA, PD-L1, PD-1, Fibroblast Activation Protein alpha (FAPalpha), the Melanoma-associated Chondroitin Sulfate Proteoglycan (MCSP), and EPCAM.
26. The prodrug of embodiment 20, wherein said prodrug comprises two polypeptide chains whose amino acid sequences respectively comprise
SEQ ID NOs: 36 and 38,
SEQ ID NOs: 37 and 38,
SEQ ID NOs: 39 and 41,
SEQ ID NOs: 40 and 41,
SEQ ID NOs: 42 and 44,
SEQ ID NOs: 43 and 44,
SEQ ID NOs: 45 and 47, or
SEQ ID NOs: 46 and 47.
   27. The prodrug of embodiment 18, comprising two heavy chain polypeptides whose amino acid sequences comprise SEQ ID NOs: 48 and 49, respectively; and a light chain comprises SEQ ID NO: 50 or 51.
   28. The prodrug of embodiment 18, comprising two heavy chain polypeptides whose amino acid sequences comprise SEQ ID NOs: 109 and 110, respectively; and a light chain comprises SEQ ID NO: 50.
   29. The prodrug of embodiment 18, comprising two heavy chain polypeptides whose amino acid sequences comprise SEQ ID NOs: 111 and 112, respectively; and a light chain comprises SEQ ID NO: 92.
   30. The prodrug of embodiment 18, wherein the carrier moiety is an antibody or antigen-binding fragment thereof that binds to FAPα or 5T4; and optionally the prodrug further comprises two or three ectodomains of a tumor necrosis factor (TNF) ligand family member or 4-1BB ligand, or fragments thereof.
   31. The prodrug of embodiment 18, wherein the carrier moiety is an antibody or antigen-binding fragment thereof that binds to CTLA4, wherein the antibody or antigen-binding fragment thereof comprises a light chain CDR domain sequences as derived from SEQ ID NO: 113, and heavy chain CDR domain sequences as derived from SEQ ID NO: 114.
   32. The prodrug of embodiment 18, wherein the carrier moiety is a bispecific antibody which binds to both EGFR and CMET.
   33. A pharmaceutical composition comprising the prodrug of any one of embodiments 1-33 and a pharmaceutically acceptable excipient.
   34. A polynucleotide or polynucleotides encoding the prodrug of any one of embodiments 1-33.
   35. An expression vector or vectors comprising the polynucleotide or polynucleotides of embodiment 35.
   36. A host cell comprising the vector(s) of embodiment 36.
   37. The host cell of embodiment 37, wherein the gene(s) encoding uPA, MMP2, and/or MMP9 are knocked out in the host cell.
   38. A method of making the prodrug of any one of embodiments 1-33, comprising
culturing the host cell of embodiment 37 or 38 under conditions that allow expression of the prodrug, wherein the host cell is a mammalian cell, and
isolating the prodrug.
   39. A method of treating a cancer or an infectious disease, or stimulating the immune system, in a patient in need thereof, comprising administering to the patient a therapeutically effective amount of the pharmaceutical composition of embodiment 34.
   40. A prodrug of any one of embodiments 1-33 for use in treating a cancer or an infectious disease, or stimulating the immune system, in a patient in need thereof.
   41. Use of a prodrug of any one of embodiments 1-33 for the manufacture of a medicament for treating a cancer or an infectious disease, or stimulating the immune system, in a patient in need thereof.
   42. The method of embodiment 40, the prodrug for use of embodiment 41, or the use of embodiment 38, wherein the patient has HIV, HBV, HCV, or HPV infection; or a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer.

In order that this invention may be better understood, the following examples are set forth. These examples are for purposes of illustration only and are not to be construed as limiting the scope of the invention in any manner.

### EXAMPLES

### Example 1: Transient Transfection of the IL-21 Prodrugs Using HEK293 Cells

Expression plasmids were co-transfected into 3×10⁶ cell/ml freestyle HEK293 cells at 2.5-3 μg/ml using PEI (polyethylenimine). For Fc-based IL-21 prodrugs (A and B), the Fc-IL-21 fusion polypeptide and the Fc-masking moiety fusion polypeptide were in a 1:2 ratio. For antibody-based IL-21 prodrugs, the knob heavy chain (containing IL-21 agonist polypeptide) and hole heavy chain (containing the masking moiety) and the light chain DNA were in a 2:1:2 molar ratio. The cell cultures were harvested 6 days after transfection by centrifuging at 9,000 rpm for 45 min followed by 0.22 μM filtration.

Two IL-21 Prodrugs (A and B) were expressed. Their corresponding controls, the Fc-IL-21 fusion molecules without the masking moiety, were also expressed. The sequence ID NOs are listed in Table 1.

TABLE 1
Sequence Information of the samples.

| Prodrug | SEQ ID for | SEQ ID for Fc- | SEQ ID for |
|---|---|---|---|
| or Control | Fc-IL-21 Fusion | Masking Moiety | the Fc |
| Name | Polypeptide | Fusion Polypeptide | Polypeptide |
| IL-21 Prodrug | SEQ ID NO: 94 | SEQ ID NO: 38 | — |
| A | | | |
| Fc-IL-21 | SEQ ID NO: 94 | — | SEQ ID NO: 96 |
| Control 1 | | | |
| IL-21 Prodrug | SEQ ID NO: 95 | SEQ ID NO: 38 | — |
| B | | | |
| Fc-IL-21 | SEQ ID NO: 94 | — | SEQ ID NO: 96 |
| Control 2 | | | |

### Example 2: Transient Transfection of ExpiCHO-S Cells

Expression plasmids were co-transfected into 6×10⁶ cell/ml ExpiCHO-S cells at 1-2 μg/ml using Expifectamine CHO Reagant. For PD-1 antibody-IL-21 fusion molecules, the ratios for the knob heavy chain:light chain:hole light chain are 1:2: 2. The cell cultures were harvested 7 days after transfection by centrifuging at 12,000 rpm for 40 min followed by 0.45 μM filtration.”

### Example 3: Purification of the Fc-Based IL-21 Prodrugs

The purifications of the proteins of the Fc-based IL-21 prodrugs A and B were carried out by using three chromatography steps: Protein A Affinity, Capto Adhere (Flow-through mode), and Capto SP ImpRes. Briefly, the supernatant of the transient expression cell culture was loaded onto a Protein A column, which was equilibrated with 25 mM Tris-HCl, 30 mM NaCl, pH 7.8 (buffer A) before applying the sample. The column was washed with 5-column volumes of buffer A and the bound protein was eluted with 50 mM acetic acid, pH 3.6. The pH of the eluted protein was adjusted to 5.2 using 1 M Tris-base and loaded onto a Capto Adhere column, which was equilibrated with 50 mM acetic acid, 30 mM NaCl, pH 5.2 (buffer B). The flow-through was collected and further loaded onto a buffer B equilibrated Capto SP ImpRes column. The column was washed with 5-column volumes of buffer B, and the bound protein was eluted with a 30-column volume gradient from 0% to 100% of 50 mM acetic acid, 1 M NaCl, pH 5.2 (buffer C). The eluted samples from each step were analyzed by HPLC-SEC. The fractions of the Capto SP ImpRes step with aggregation less than 10% were pooled for the further analyses.

### Example 4: SEC-HPLC Analysis

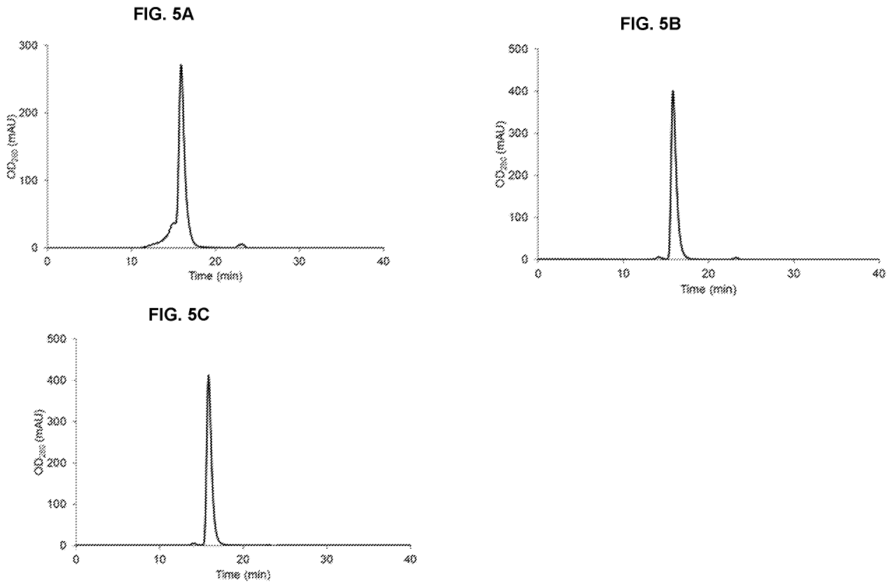

SEC-HPLC was carried out using an Agilent 1100 Series of HPLC system with a TSKgel G3000SWXL column (7.8 mm IDX 30 cm, 5 μm particle size) ordered from Tosoh Bioscience. A sample of up to 100 μl was loaded. The column was run with a buffer containing 200 mM K₃PO₄, 250 mM KCl, pH 6.5. The flow rate was 0.5 ml/min. The column was run at room temperature. The protein elution was monitored both at 220 nm and 280 nm. The in-process pools of the IL-21 Prodrug A were analyzed by the SEC-HPLC. FIG. 5A shows the assay result for the Protein A column pool; FIG. 5B shows the assay result for the Capto Adhere column pool; and FIG. 5C shows the assay result for the Capto Sp ImpRes column pool. The data show that the Protein A column purified prodrug comprised a main peak with some aggregates (FIG. 5A). It had a main peak purity of about 80% as analyzed by SEC-HPLC. The aggregates were significantly reduced by the subsequent chromatography steps and the Capto SP Impres pool showed a product purity of over 98% as tested by SEC-HPLC (FIG. 5C).

### Example 5: SDS-PAGE Analysis

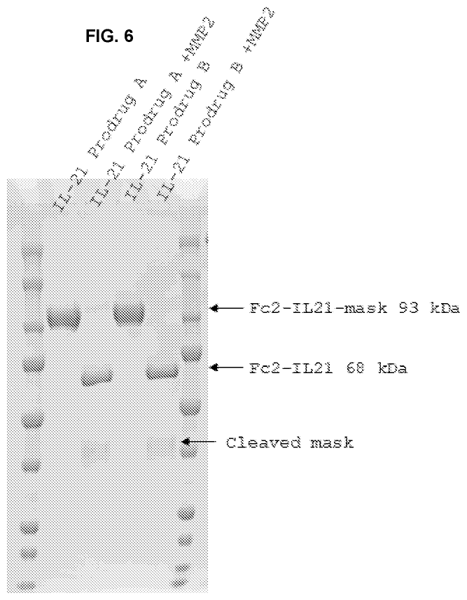

10 μl of the culture supernatants or 10-20 μg of purified protein samples were mixed with Bolt™ LDS Sample Buffer (Novex) with or without reduce reagents. The samples were heated at 70° C. for 3 min and then loaded to a NuPAGE™ 4-12% BisTris Gel (Invitrogen). The gel was run in NuPAGE™ MOPS SDS Running buffer (Invitrogen) at 200 Volts for 40 min and then stained with Coomassie. The purified samples of Prodrugs A and B together with the ones treated with the protease MMP-2 (see below) were analyzed by the SDS-PAGE analysis, as shown in FIG. 6. The data show that the masking moieties of both Prodrug A and Prodrug B were completely removed by the protease digestion, and that the activated molecules migrated at the expected molecular weights.

### Example 6: Proteolytic Treatment

The proteases, human MMP2, human MMP9, mouse MMP2 and mouse MMP9 were purchased from R&D systems. The protease digestion was carried out by incubating 10 μg-50 of prodrugs with 1 μg of human MMP2, human MMP9, mouse MMP2 or mouse MMP9 in the HBS buffer (20 mM HEPES, 150 mM NaCl₂, pH 7.4) containing 2 mM CaCl2 and 10 μM ZnCl₂ at 37° C. for 12 hours. The prodrugs prior to and after digestion were analyzed by SDS-PAGE (FIG. 6) and the cell-based activity assay (see below).

### Example 7: Cell-Based Activity Assay

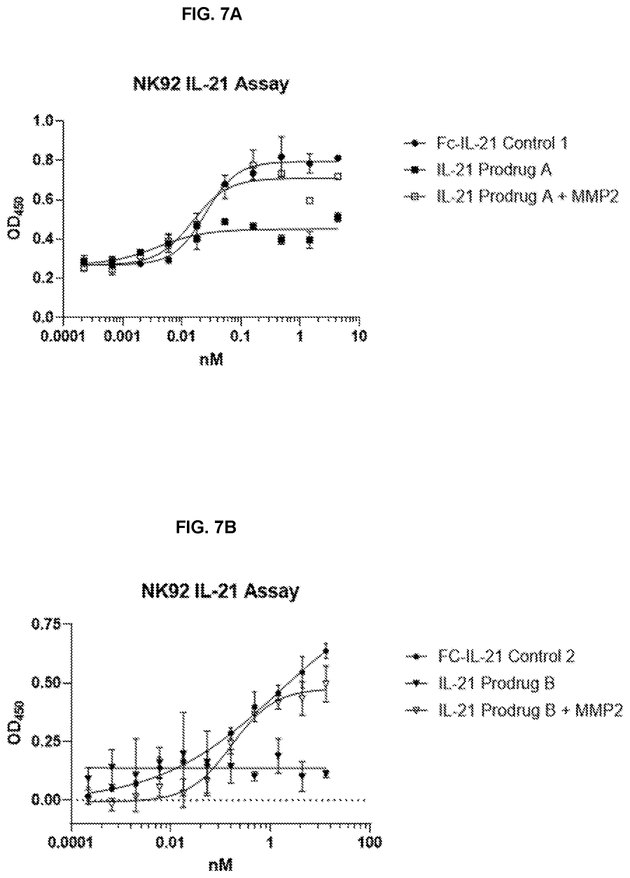

The prodrugs prior to and the protease digestion and the control samples were tested by the cell-based activity assay. Briefly, NK92 cells were grown in the RPMI 1640 medium supplemented with L-glutamine, 10% fetal bovine serum, 10% non-essential amino acids, 10% sodium pyruvate, and 55 μM beta-mercaptoethanol. NK92 cells were non-adherent and maintained at 1×10⁵-1×10⁶ cells/ml in medium with 100 ng/ml of IL-2. Generally, cells were split twice per week. For bioassays, it was best to use cells no less than 48 hours after passage. IL-21 functional activity was determined by culturing NK92 cells at 5×10⁴ cells/well with serial dilutions of the samples in the presence of a constant amount of IL-2 for 2 days. Supernatants are then assayed for Interferon-γ by ELISA. The results are shown in FIGS. 7A and 7B. The data show that the bioassay activities of the prodrugs were significantly enhanced by the protease MMP-2 treatment.

The protease-treated (or activated) prodrugs showed similar activities as those of the control Fc-IL-21 fusion molecules, even though the masking moiety, i.e., IL-21Rα ECD, was not removed from the protease-treated sample. Surprisingly, the presence of the masking moiety released by the protease digestion did not seem to interfere with the IL-21 bioassay, given that IL-21 binds to IL-21Rα with very high affinity (a K_{D} of ˜70 pM).

### Example 8: Anti-PD-1 Antibody-IL-21 Prodrug Fusion Molecules

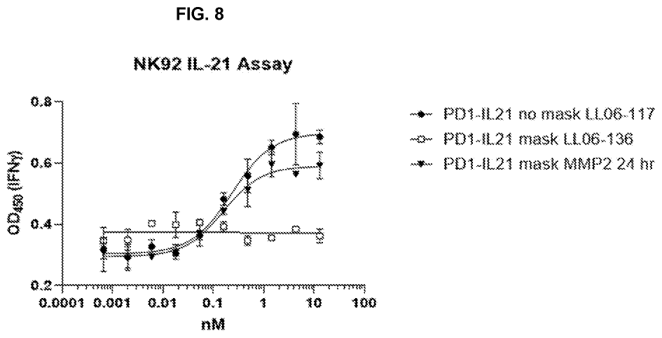

An Anti-PD-1 antibody-based IL-21 prodrug was constructed with two identical light chains (with an amino acid sequence as shown in SEQ ID NO: 50). A first heavy chain polypeptide chain (with an amino acid sequence as shown in SEQ ID NO: 48) and a second heavy chain polypeptide chain (with an amino acid sequence as shown SEQ ID NO: 49). The molecule was expressed and purified. As a control, the anti-PD-1 antibody-IL-21 fusion molecule without the mask was also expressed and purified. The cell-based activity assays for the cytokine prodrug prior to and after activation were tested using the same method as described above. The data are shown in FIG. 8. The results show that the IL-21 activity prior to activation was minimal. After activation, the IL-21 activity was similar as that of the IL-21 in the PD-1-IL-21 fusion molecule.

The activity of the anti-PD-1 antibody was also tested prior to and after the activation using the PD1/PD-L1 blockade reporter assay. The ability of anti-PD-1 antibody to block PD-L1 mediated PD1 signaling was measured using two engineered cell lines. The first is a CHO-K1 cell line (CHO-K1/TCRA/PD-L1, BPS Bioscience cat #60536) expressing both human PD-L1 and a T cell receptor activator. The second cell line (PD1/NFAT, BPS Bioscience cat #60535) is a Jurkat T cell line expressing PD-1 and an NFAT firefly luciferase reporter. The T cell receptor activator on the CHO-K1 cells will activate the Jurkat cells resulting in expression of the NFAT luciferase reporter. However, since the CHO-K1 cells also express PD-L1, signaling via PD-1 results in inhibition of NFAT activation. Blocking the PD-L1/PD-1 interaction will restore NFAT activation and luciferase activity.

To carry out the assay, CHO-K1/TCRA/PD-L1 cells were seeded in 96-well flat bottom plates at 35,000 cells/well in 50 μL assay medium (RPMI-1640, 10% fetal bovine serum, non-essential amino acids, 2-mercaptoethanol, and gentamicin) in 96-well white walled, flat bottom plates. After overnight culture, the culture medium was removed and samples and standards were added at 2× concentration in 50 μL/well. Plates were incubated 20 minutes, and 40,000 PD1/NFAT cells were added to each well in 50 μL. Plates were incubated 6 hours at 37° C. Plates were cooled to room temperature for 5 minutes, and 100 μL/well luciferase reagent (Pierce Firefly Luc One-Step Glow Assay Kit, Thermo Scientific cat #16197) was added. Plates were incubated for 15 minutes, then luminescence was measured on a luminometer.

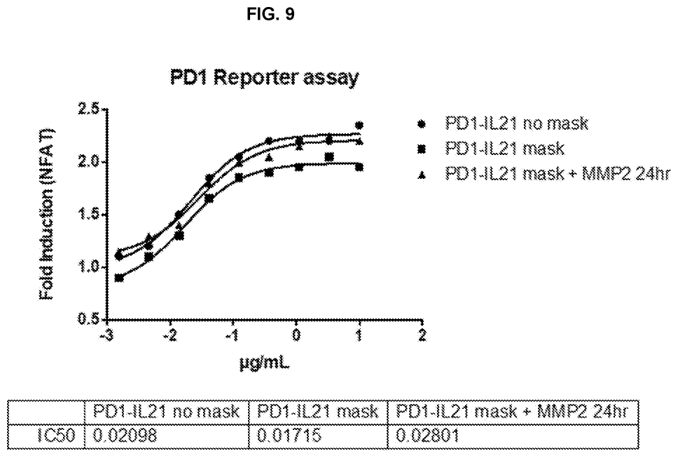

The assay results (FIG. 9) indicate that the anti-PD-1 antibodies in the fusion molecules had retained their biological functionality.

### Example 9: Additional Anti-PD-1 Antibody-IL-21 Prodrug Fusion Molecules

An Anti-PD-1 antibody-based IL-21 prodrug was constructed with two identical light chains (with an amino acid sequence as shown in SEQ ID NO: 50), a first heavy chain polypeptide chain (with an amino acid sequence as shown in SEQ ID NO: 48) and a second heavy chain polypeptide chain (with an amino acid sequence as shown SEQ ID NO: 49). The molecule was transiently expressed and purified (Lot #PW04-38). A second PD-1 antibody-based IL-21 prodrug with the scFv as the masking moiety was also expressed and purified (Lot #PW05-68). It comprises two identical light chains (with an amino acid sequence as shown in SEQ ID NO: 50), a first heavy chain polypeptide chain (with an amino acid sequence as shown in SEQ ID NO: 48) and a second heavy chain polypeptide chain (with an amino acid sequence as shown SEQ ID NO: 133). In addition, as a control, the anti-PD-1 antibody-IL-21 fusion molecule without the mask was also expressed and purified (Lot #PW05-67). It comprises two identical light chains (with an amino acid sequence as shown in SEQ ID NO: 50), a first heavy chain polypeptide chain (with an amino acid sequence as shown in SEQ ID NO: 48) and a second heavy chain polypeptide chain (with an amino acid sequence as shown SEQ ID NO: 134). Further, a second control, the anti-PD-1 antibody-IL-21 mutein (R9ER76A) fusion molecule without the mask was also expressed and purified (Lot #PW09-02), which comprises two identical light chains (with an amino acid sequence as shown in SEQ ID NO: 50), a first heavy chain polypeptide chain (with an amino acid sequence as shown in SEQ ID NO: 135) and a second heavy chain polypeptide chain (with an amino acid sequence as shown SEQ ID NO: 134).

In addition, PD-1 antibody-based IL-21 prodrug without cleavage peptide linker was also expressed and purified. The prodrug of Lot #PW09-44 comprises two identical light chains (with an amino acid sequence as shown in SEQ ID NO: 50), a first heavy chain polypeptide chain (with an amino acid sequence as shown in SEQ ID NO: 117) and a second heavy chain polypeptide chain (with an amino acid sequence as shown SEQ ID NO: 130).

### Example 10: Binding Assay

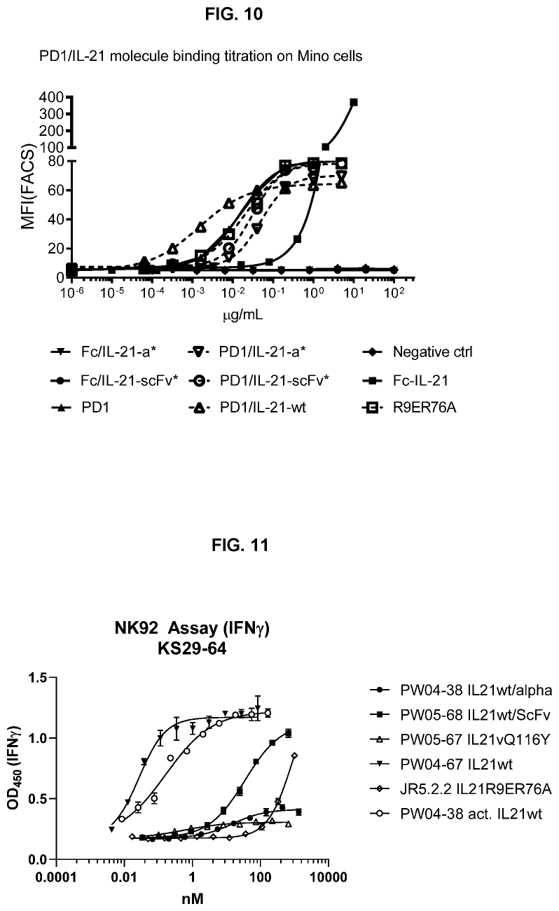

The binding of the prodrug molecules and several control molecules to the mino cells were tested by FACS. The results on FIG. 10 show that both the PD-1 antibody and the Fc-IL-21 fusion molecule were able to bind to the mino cells, indicating that the mino cells express both the PD-1 and the receptors for IL-21. The results showed that both the Fc-based IL-21 prodrug molecules had no binding to the cells, suggesting the IL-21 cytokine moieties have been masked by the corresponding masking moiety. However, the PD-1 antibody-based IL-21 prodrug molecules and fusion molecules were able to bind to the mino cells.

### Example 11: NK92 Cell-Based Activity Assay of the PD-1-Antibody-Based Prodrugs

The antibody-based prodrugs prior to protease digestion and the control samples were tested by the cell-based activity assay. Briefly, NK92 cells were grown in the RPMI-1640 medium supplemented with L-glutamine, 10% fetal bovine serum, 10% non-essential amino acids, 10% sodium pyruvate, and 55 μM beta-mercaptoethanol. NK92 cells were non-adherent and maintained at 1×10⁵-1×10⁶ cells/ml in medium with 100 ng/ml of IL-2. Generally, cells were split twice per week. For bioassays, it was best to use cells no less than 48 hours after passage. IL-21 functional activity was determined by culturing NK92 cells at 5×10⁴ cells/well with serial dilutions of the samples in the presence of a constant amount of IL-2 for 2 days. Supernatants were then assayed for interferon-γ by ELISA. The results are shown in FIG. 11. The data show that without activation, the prodrug molecule with the IL-21α-ECD (Lot #PW04-38) had minimum activity; while the prodrug with a scFv as the masking moiety (Lot #PW05-68) had an activity ˜1000 times lower than the one without the masking moiety (PW04-67). The data show that the bioassay activities of the prodrugs were significantly enhanced by the protease MMP2 treatment.

### Example 12: Mino IL-21 Viability Assay

The Mino cell viability assay is carried out following the protocol below:
a) Perform serial dilutions of test articles in 50 uL assay medium (RPMI 1640, 10% Fetal Bovine Serum, NEAA, sodium pyruvate, 55 μM b-mercaptoethanol) in 96 well tissue culture plate.
b) Add 20,000 Mino cells/well in 50 μL assay medium.
c) Culture for 2 or 3 days.
d) Add 100 μL/well Cell Titer Glo (Promega). Cell Titer-Glo provides a measure of cell viability by providing a quantitative assessment of ATP.
e) Measure luminescence.

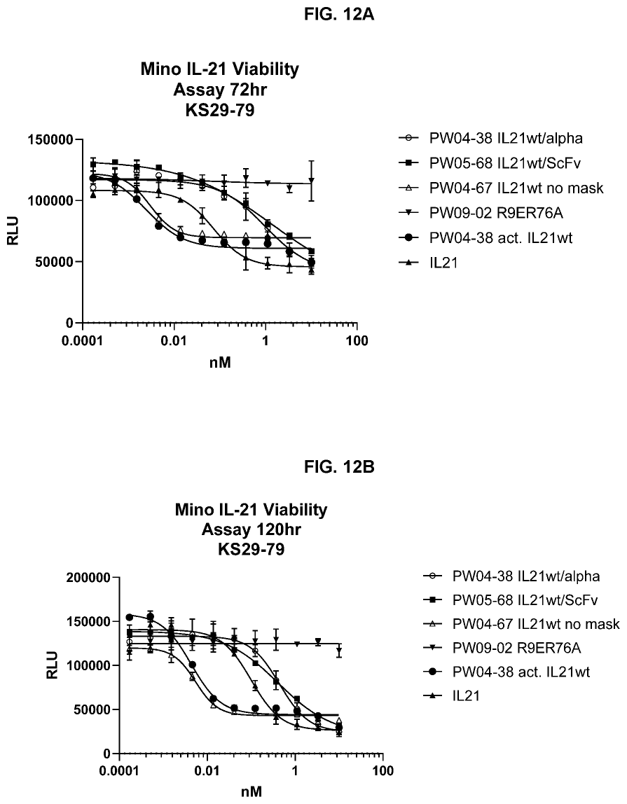

The mino viability assay results are shown in FIGS. 12A and 12B. Surprisingly, prodrugs (Lots #PW04-38 and PW05-68) had significant activities prior to activation, while the control molecule (PD-1 antibody-IL-21R9E/R76A fusion molecule, Lot #PW09-02) had no or little activity. Mino cells express PD-1. While not wishing to be bound by theory, it is hypothesized that Mino cells express both PD-1 and receptors for IL-21 and the prodrugs were activated through “cis-biding,” i.e., through binding to both the PD-1 and the IL-21 receptor(s). Cis-binding of the PD-1 antibody to the PD-1 antigen on the cell surface and the cytokine to its receptor on the same cell surface may have unraveled the masking effect of the masking moiety. It is therefore possible that prodrugs without cleavable peptide linker may be “activated” in a disease site such as a tumor because the local immune cells may express both the antigen targeted by the carrier and the receptor(s), which bind the cytokine moiety (IL-21).

### Example 13: In Vivo Efficacy Study with a Syngeneic Tumor Model

Six-week old Balb/c mice (Taconic Biosciences) are injected subcutaneously with 1×10⁶ CT26/18.2 cells. After 7 days, tumors are measured using digital calipers and tumor volume was calculated (V=(ab2)p/6, where b is the shorter of 2 dimensions measured). Mice are then randomized into treatment groups such that all groups have approximately the same mean tumor size (˜100 mm³). Mice were then treated with placebo or test article at 0.5-5 mg/Kg in 100 μl via intraperitoneal injection. Dosing was performed on days 7, 9, 11, 13, 15 and 18. Tumors were measured every 2-3 days, and mice were sacrificed when tumors reached 2000 mm³.

The above non-limiting examples are provided for illustrative purposes only in order to facilitate a more complete understanding of the disclosed subject matter. These examples should not be construed to limit any of the embodiments described in the present specification, including those pertaining to the antibodies, pharmaceutical compositions, or methods and uses for treating cancer, a neurodegenerative or an infectious disease.

### SEQUENCES

In the sequences below, boxed residues indicate mutations. Underlines in cleavable linkers indicate protease substrate sequences. Italicized residues represent the signal peptide.

SEQ ID NO: 1- human IL-21
QGQDRHMIRM RQLIDIVDQL KNYVNDLVPE FLPAPEDVET NCEWSAFSCF QKAQLKSANT
GNNERIINVS IKKLKRKPPS TNAGRRQKHR LTCPSCDSYE KKPPKEFLER FKSLLQKMIH
QHLSSRTHGS EDS
SEQ ID NO: 2- IL-21 Mutein A

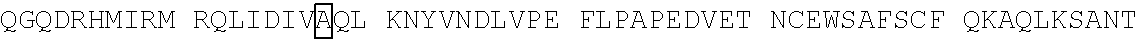

GNNERIINVS IKKLKRKPPS TNAGRRQKHR LTCPSCDSYE KKPPKEFLER FKSLLQKMIH
QHLSSRTHGS EDS
SEQ ID NO: 3- IL-21 Mutein B

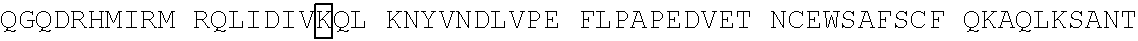

GNNERIINVS IKKLKRKPPS TNAGRRQKHR LTCPSCDSYE KKPPKEFLER FKSLLQKMIH
QHLSSRTHGS EDS
SEQ ID NO: 4- IL-21 Mutein C
QGQDRHMIRM RQLIDIVDQL KNYVNDLVPE FLPAPEDVET NCEWSAFSCF QKAQLKSANT

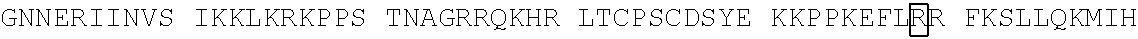

QHLSSRTHGS EDS
SEQ ID NO: 5- IL-21 Mutein D

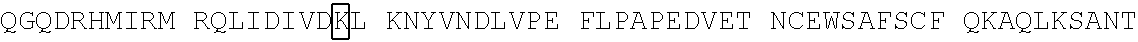

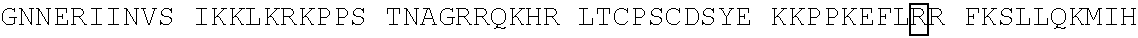

QHLSSRTHGS EDS
SEQ ID NO: 6- IL-21 receptor extracellular domain (source:
uniprot.org/uniprot/Q9HBE5)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SDYEDPAFYM LKGKLQYELQ YRNRGDPWAV SPRRKLISVD SRSVSLLPLE FRKDSSYELQ
VRAGPMPGSS YQGTWSEWSD PVIFQTQSEE LKE
SEQ ID NO: 7- Human IL-21 Receptor Gamma Subunit Extracellular Domain
LNITILTPNG NEDTTADFFL TTMPTDSLSV STLPLPEVQC FVFNVEYMNC TWNSSSEPQP TNLTLHYWYK
NSDNDKVQKC SHYLFSEEIT SGCQLQKKEI HLYQTFVVQL QDPREPRRQA TQMLKLQNLV IPWAPENLTL
HKLSESQLEL NWNNRFLNHC LEHLVQYRTD WDHSWTEQSV DYRHKFSLPS VDGQKRYTFR VRSRFNPLCG
SAQHWSEWSH PIHWGSNTSK ENPFLFALEA
SEQ ID NO: 8- Human IL-2 amino acid sequence
APTSSSTKKT QLQLEHLLLD LQMILNGINN YKNPKLTRML TFKFYMPKKA TELKHLQCLE EELKPLEEVL
NLAQSKNFHL RPRDLISNIN VIVLELKGSE TTFMCEYADE TATIVEFLNR WITFCQSIIS TLT
SEQ ID NO: 9- Human IL-15 amino acid sequence
NWVNVISDLK KIEDLIQSMH IDATLYTESD VHPSCKVTAM KCFLLELQVI SLESGDASIH DTVENLIILA
NNSLSSNGNV TESGCKECEE LEEKNIKEFL QSFVHIVQMF INT
SEQ ID NO: 10- Human IL-15 receptor alpha subunit sushi domain
ITCPPPMSVE HADIWVKSYS LYSRERYICN SGFKRKAGTS SLTECVLNKA TNVAHWTTPS LKCIRDPALV
HQRPA
SEQ ID NO: 11-17- MMP-2/MMP-9 Cleavable peptide linkers
GPLGVR (SEQ ID NO: 11)
PLGMWSR (SEQ ID NO: 12)
PLGLWAR (SEQ ID NO: 13)
PQGIAGQR (SEQ ID NO: 14)
PLGLAG (SEQ ID NO: 15)
LALGPR (SEQ ID NO: 16)
GGPLGMLSQS (SEQ ID NO: 17)
SEQ ID NO: 18-26- urokinase plasminogen activator (uPA) Cleavable peptide
linkers
GGGGRRGGS (SEQ ID NO: 18)
TGRGPSWV (SEQ ID NO: 19)
SARGPSRW (SEQ ID NO: 20)
TARGPSFK (SEQ ID NO: 21)
TARGPSW (SEQ ID NO: 22)
GGWHTGRN (SEQ ID NO: 23)
HTGRSGAL (SEQ ID NO: 24)
PLTGRSGG (SEQ ID NO: 25)
LTGRSGA (SEQ ID NO: 26)
SEQ ID NO: 27- Human CCL19 amino acid sequence
TNDAEDCC LSVTQKPIPG YIVRNFHYLL IKDGCRVPAV VFTTLRGRQL CAPPDQPWVE RIIQRLQRTS
AKMKRRSS
SEQ ID NO: 28- Human IL-7 amino acid sequence
DCDIEGKDGK QYESVLMVSI DQLLDSMKEI GSNCLNNEFN FFKRHICDAN KEGMFLFRAA RKLRQFLKMN
STGDFDLHLL KVSEGTTILL NCTGQVKGRK PAALGEAQPT KSLEENKSLK EQKKLNDLCF LKRLLQEIKT
CWNKILMGTK EH
SEQ ID NO:29-33 Peptide Linker
GGGGS (SEQ ID NO: 29)
GGGGSGGGGS (SEQ ID NO: 30)
GGGGSGGGGS GGGGS (SEQ ID NO: 31)
GGGGSGGGGX GGGGSGGGGS (SEQ ID NO: 32), X = A or N
GGGGSGGGGX GGGGYGGGGS (SEQ ID NO: 33), X = S, A or N, and Y = A or N
SEQ ID NO: 34- IgG1 Fc
DKTHTCPPCP APELLGGPSV FLFPPKPKDT LMISRTPEVT CVVVDVSHED PEVKFNWYVD GVEVHNAKTK
PREEQYNSTY RVVSVLTVLH QDWLNGKEYK CKVSNKALPA PIEKTISKAK GQPREPQVYT LPPSRDELTK
NQVSLTCLVK GFYPSDIAVE WESNGQPENN YKTTPPVLDS DGSFFLYSKL TVDKSRWQQG NVFSCSVMHE
ALHNHYTQKS LSLSPGK
SEQ ID NO: 35- IgG4 Fc

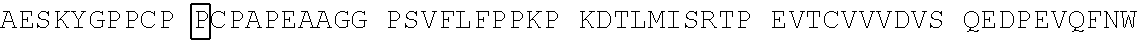

YVDGVEVHNA KTKPREEQFN STYRVVSVLT VLHQDWLNGK EYKCKVSNKG LPSSIEKTIS
KAKGQPREPQ VYTLPPSQEE MTKNQVSLTC LVKGFYPSDI AVEWESNGQP ENNYKTTPPV
LDSDGSFFLY SRLTVDKSRW QEGNVFSCSV MHEALHNHYT QKSLSLSLGK
SEQ ID NO: 36- Fc- IL-21 with knobs mutations

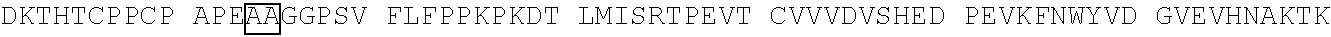

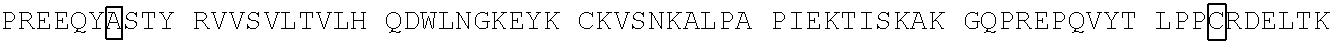

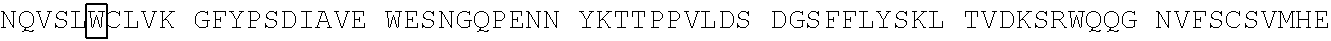

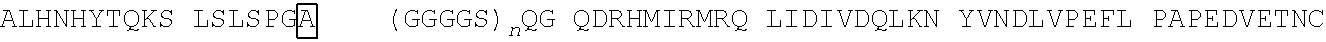

EWSAFSCFQK AQLKSANTGN NERIINVSIK KLKRKPPSTN AGRRQKHRLT CPSCDSYEKK PPKEFLERFK
SLLQKMIHQH LSSRTHGSED S; wherein n = 0, 1, 2, 3, 4, or 5.
SEQ ID NO: 37- Fc- IL-21 mutein with knobs mutations

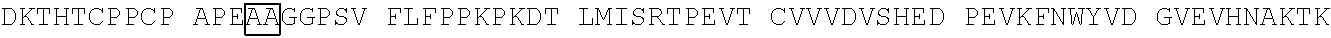

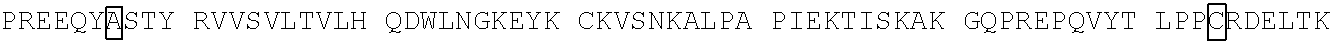

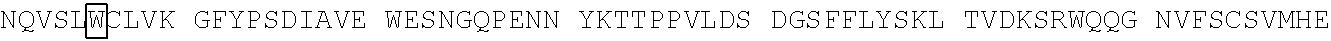

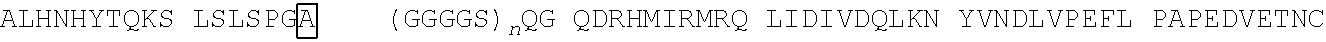

EWSAFSCFQK AQLKSANTGN NERIINVSIK KLKRKPPSTN AGRRQKHRLT CPSCDSYEKK PPKEFLRRFK
SLLQKMIHQH LSSRTHGSED S; wherein n = 0, 1, 2, 3, 4, or 5.
SEQ ID NO: 38- Fc- IL-21 Receptor ECD with holes mutations

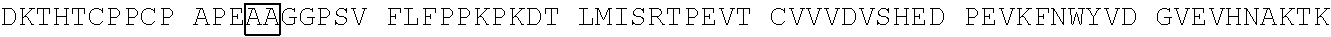

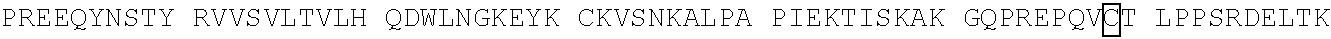

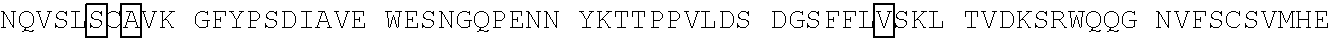

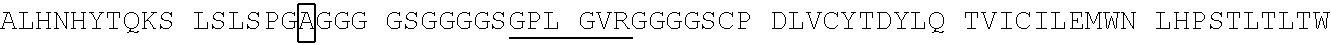

QDQYEELKDE ATSCSLHRSA HNATHATYTC HMDVFHFMAD DIFSVNITDQ SGNYSQECGS FLLAESIKPA
PPFNVTVTFS GQYNISWRSD YEDPAFYMLK GKLQYELQYR NRGDPWAVSP RRKLISVDSR SVSLLPLEFR
KDSSYELQVR AGPMPGSSYQ GTWSEWSDPV IFQTQSEELK E
SEQ ID NO: 39- IL-21-Fc with knobs mutations
QGQDRHMIRM RQLIDIVDQL KNYVNDLVPE FLPAPEDVET NCEWSAFSCF QKAQLKSANT GNNERIINVS
IKKLKRKPPS TNAGRRQKHR LTCPSCDSYE KKPPKEFLER FKSLLQKMIH QHLSSRTHGS EDS(GGGGS)ₙ
DKTHTCPPCP APELLGGPSV FLFPPKPKDT LMISRTPEVT CVVVDVSHED PEVKFNWYVD GVEVHNAKTK

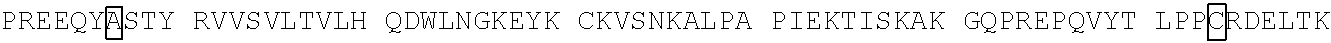

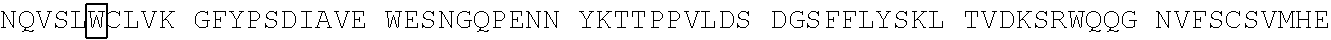

ALHNHYTQKS LSLSPGK; wherein n = 0, 1, 2, 3, 4, or 5.
SEQ ID NO: 40- IL-2-Fc mutein with knobs mutations
QGQDRHMIRM RQLIDIVDKL KNYVNDLVPE FLPAPEDVET NCEWSAFSCF QKAQLKSANT GNNERIINVS
IKKLKRKPPS TNAGRRQKHR LTCPSCDSYE KKPPKEFLRR FKSLLQKMIH QHLSSRTHGS EDS(GGGGS)n
DKTHTCPPCP APELLGGPSV FLFPPKPKDT LMISRTPEVT CVVVDVSHED PEVKFNWYVD GVEVHNAKTK

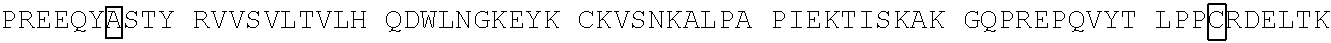

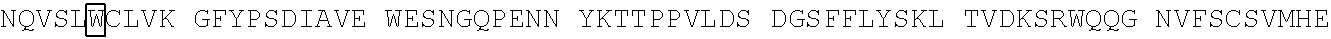

ALHNHYTQKS LSLSPGK; wherein n = 0, 1, 2, 3, 4, or 5.
SEQ ID NO: 41- IL-21Rα ECD-Fc with holes mutations
CPDLVCYTDY LQTVICILEM WNLHPSTLTL TWQDQYEELK DEATSCSLHR SAHNATHATY TCHMDVFHFM
ADDIFSVNIT DQSGNYSQEC GSFLLAESIK PAPPFNVTVT FSGQYNISWR SDYEDPAFYM LKGKLQYELQ
YRNRGDPWAV SPRRKLISVD SRSVSLLPLE FRKDSSYELQ VRAGPMPGSS YQGTWSEWSD PVIFQTQSEE
LKEGGGGSGG GGSGPLGVRG GGGSDKTHTC PPCPAPELLG GPSVFLFPPK PKDTLMISRT PEVTCVVVDV

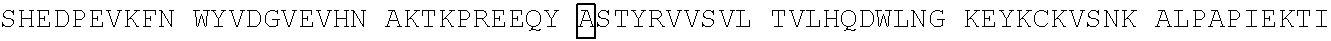

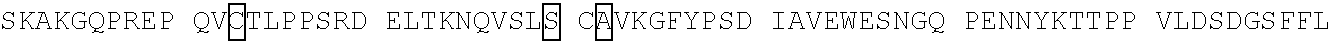

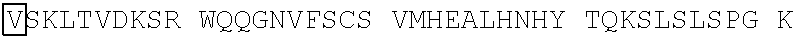

SEQ ID NO: 42- IL-15-Sushi- Fc- IL-21 with knobs mutations
NWVNVISDLK KIEDLIQSMH IDATLYTESD VHPSCKVTAM KCFLLELQVI SLESGDASIH DTVENLIILA
NNSLSSNGNV TESGCKECEE LEEKNIKEFL QSFVHIVXMF INT(GGGGS)ₙ₁ ITCPPPMSVE HADIWVKSYS
LYSRERYICN SGFKRKAGTS SLTECVLNKA TNVAHWTTPS LKCIRDPALV HQRPA (GGGGS)ₙ₂DK
THTCPPCPAP EAAGGPSVFL FPPKPKDTLM ISRTPEVTCV VVDVSHEDPE VKFNWYVDGV EVHNAKTKPR

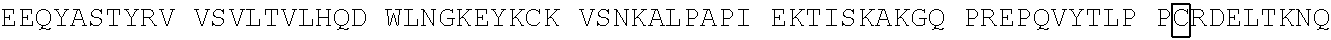

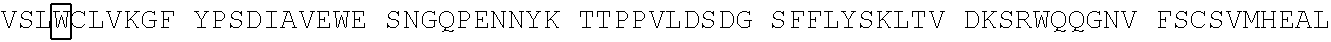

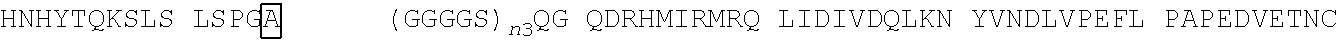

EWSAFSCFQK AQLKSANTGN NERIINVSIK KLKRKPPSTN AGRRQKHRLT CPSCDSYEKK PPKEFLERFK
SLLQKMIHQH LSSRTHGSED S; wherein n1 = 0, 1, 2, 3, 4, or 5; n2 = 0, 1, 2, 3,
4, or 5; n3 = 0, 1, 2, 3, 4, or 5; and X is an amino acid residue selected
from Q and E.
SEQ ID NO: 43- IL2 analog- IL-21 mutein with knobs mutations
APASSSTKKT QLQLEHLLLD LQMILNGINN YKNPKLTSML TKKFAMPKKA TELKHLQCLE EALKPLEEVL
NLTQSKNFHL RPRDLISNIN VIVLELKGSE TTFMCEYADE TATIVEFLNR WITFSXSIIS TLT(GGGGS)ₙ₁
DKTHTCPPCP APEAAGGPSV FLFPPKPKDT LMISRTPEVT CVVVDVSHED PEVKFNWYVD GVEVHNAKTK

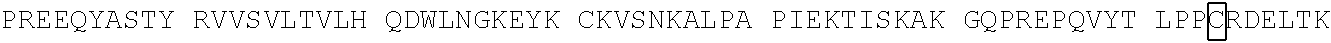

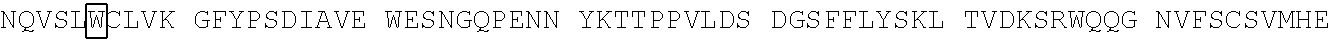

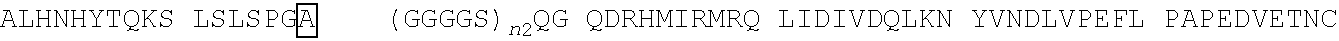

EWSAFSCFQK AQLKSANTGN NERIINVSIK KLKRKPPSTN AGRRQKHRLT CPSCDSYEKK PPKEFLRRFK
SLLQKMIHQH LSSRTHGSED S; wherein n1 = 0, 1, 2, 3, 4, or 5; an2 = 0, 1, 2, 3,
4, or 5; and X is an amino acid residue selected from Q, A, W, H and E.
SEQ ID NO: 44- IL-2Rβ ECD- Fc- IL-21Ra ECD with holes mutations
AVNGTSQFTC FYNSRANISC VWSQDGALQD TSCQVHAWPD RRRWNQTCEL LPVSQASWAC NLILGAPDSQ
KLTTVDIVTL RVLCREGVRW RVMAIQDFKP FENLRLMAPI SLQVVHVETH RCNISWEISQ ASHYFERHLE
FEARTLSPGH TWEEAPLLTL KQKQEWICLE TLTPDTQYEF QVRVKPLQGE FTTWSPWSQP LAFRTKPAAL
GKDTGGGGSG GGGSGPLGVR GGGGSDKTHT CPPCPAPEAA GGPSVFLFPP KPKDTLMISR TPEVTCVVVD
VSHEDPEVKF NWYVDGVEVH NAKTKPREEQ YNSTYRVVSV LTVLHQDWLN GKEYKCKVSN KALPAPIEKT

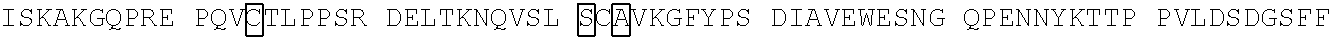

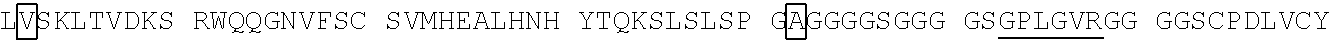

TDYLQTVICI LEMWNLHPST LTLTWQDQYE ELKDEATSCS LHRSAHNATH ATYTCHMDVF HFMADDIFSV
NITDQSGNYS QECGSFLLAE SIKPAPPFNV TVTFSGQYNI SWRSDYEDPA FYMLKGKLQY ELQYRNRGDP
WAVSPRRKLI SVDSRSVSLL PLEFRKDSSY ELQVRAGPMP GSSYQGTWSE WSDPVIFQTQ SEELKE
SEQ ID NO: 45- IL-21- Fc with knobs mutations-Sushi- IL-15
QGQDRHMIRM RQLIDIVDQL KNYVNDLVPE FLPAPEDVET NCEWSAFSCF QKAQLKSANT GNNERIINVS
IKKLKRKPPS TNAGRRQKHR LTCPSCDSYE KKPPKEFLER FKSLLQKMIH QHLSSRTHGS EDS(GGGGS)ₙ
DKTHTCPPCP APELLGGPSV FLFPPKPKDT LMISRTPEVT CVVVDVSHED PEVKFNWYVD GVEVHNAKTK

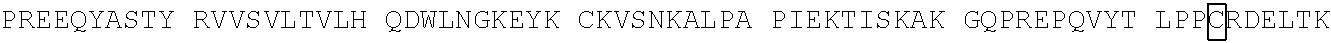

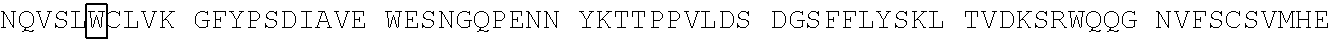

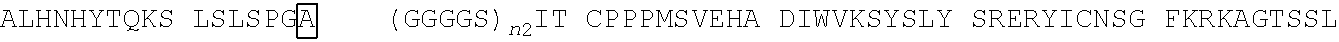

TECVLNKATN VAHWTTPSLK CIRDPALVHQ RPA(GGGGS)ₙ₃ NWVNVISDLK KIEDLIQSMH IDATLYTESD
VHPSCKVTAM KCFLLELQVI SLESGDASIH DTVENLIILA NNSLSSNGNV TESGCKECEE LEEKNIKEFL
QSFVHIVQMF INT; wherein n1 = 0, 1, 2, 3, 4, or 5; n2 = 0, 1, 2, 3, 4, or 5;
and n3 = 0, 1, 2, 3, 4, or 5.
SEQ ID NO: 46- IL-21 mutein- Fc with knobs mutations- IL-2 analog
QGQDRHMIRM RQLIDIVDKL KNYVNDLVPE FLPAPEDVET NCEWSAFSCF QKAQLKSANT GNNERIINVS
IKKLKRKPPS TNAGRRQKHR LTCPSCDSYE KKPPKEFLRR FKSLLQKMIH QHLSSRTHGS EDS(GGGGS)ₙ₁
DKTHTCPPCP APELLGGPSV FLFPPKPKDT LMISRTPEVT CVVVDVSHED PEVKFNWYVD GVEVHNAKTK

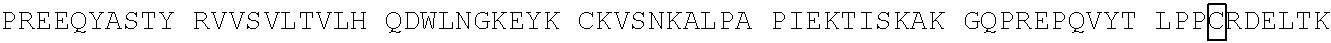

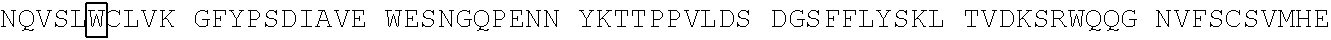

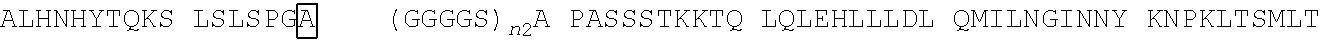

KKFAMPKKAT ELKHLQCLEE ALKPLEEVLN LTQSKNFHLR PRDLISNINV IVLELKGSET TFMCEYADET
ATIVEFLNRW ITFSQSIIST LT; wherein n1 = 0, 1, 2, 3, 4, or 5; and n2 = 0, 1, 2,
3, 4, or 5.
SEQ ID NO: 47- IL-21Rα ECD- Fc with holes mutations- IL-2Rβ ECD
CPDLVCYTDY LQTVICILEM WNLHPSTLTL TWQDQYEELK DEATSCSLHR SAHNATHATY TCHMDVFHFM
ADDIFSVNIT DQSGNYSQEC GSFLLAESIK RAPPFNVTVT FSGQYNISWR SDYEDPAFYM LKGKLQYELQ
YRNRGDPWAV SPRRKLISVD SRSVSLLPLE FRKDSSYELQ VRAGPMPGSS YQGTWSEWSD PVIFQTQSEE
LKEGGGGSGG GGSGPLGVRG GGGSDKTHTC PPCPAPELLG GPSVFLFPPK PKDTLMISRT PEVTCVVVDV
SHEDPEVKFN WYVDGVEVHN AKTKPREEQY NSTYRVVSVL TVLHQDWLNG KEYKCKVSNK ALPAPIEKTI

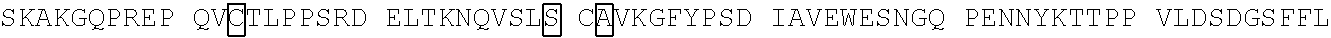

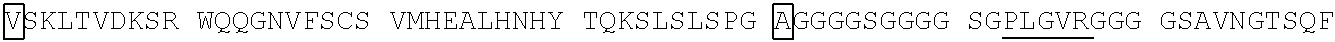

TCFYNSRANI SCVWSQDGAL QDTSCQVHAW PDRRRWNQTC ELLPVSQASW ACNLILGAPD SQKLTTVDIV
TLRVLCREGV RWRVMAIQDF KPFENLRLMA PISLQVVHVE THRCNISWEI SQASHYFERH LEFEARTLSP
GHTWEEAPLL TLKQKQEWIC LETLTPDTQY EFQVRVKPLQ GEFTTWSPWS QPLAFRTKPA ALGKDT
SEQ ID NO: 48- PD1-HC-IL21 knob mutations
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

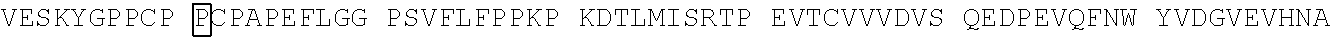

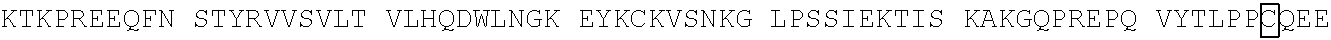

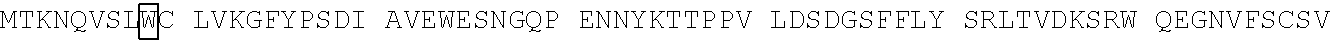

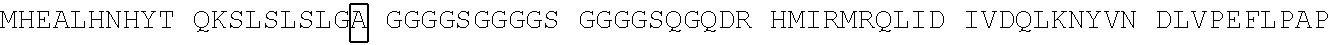

EDVETNCEWS AFSCFQKAQL KSANTGNNER IINVSIKKLK RKPPSTNAGR RQKHRLTCPS CDSYEKKPPK
EFLERFKSLL QKMIHQHLSS RTHGSEDS
SEQ ID NO: 49- PD1-HC-IL21RA ECD with hole mutations
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

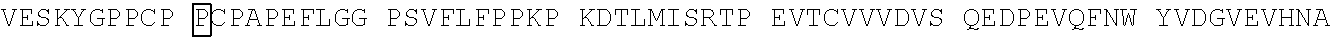

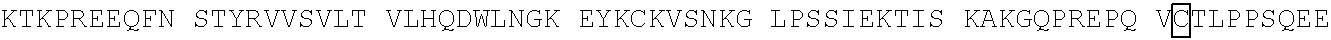

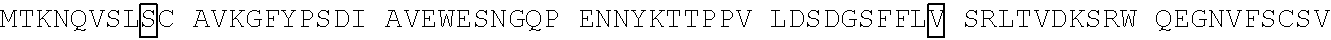

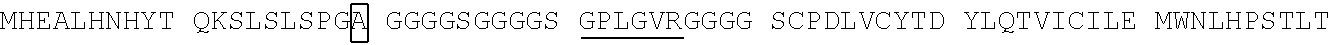

LTWQDQYEEL KDEATSCSLH RSAHNATHAT YTCHMDVFHF MADDIFSVNI TDQSGNYSQE CGSFLLAESI
KPAPPFNVTV TFSGQYNISW RSDYEDPAFY MLKGKLQYEL QYRNRGDPWA VSPRRKLISV DSRSVSLLPL
EFRKDSSYEL QVRAGPMPGS SYQGTWSEWS DPVIFQTQSE ELKE
SEQ ID NO: 50- PD1-LC
EIVLTQSPAT LSLSPGERAT LSCRASQSVS SYLAWYQQKP GQAPRLLIYD ASNRATGIPA RFSGSGSGTD
FTLTISSLEP EDFAVYYCQQ SSNWPRTFGQ GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 51- PD1-LC-IL2basal
EIVLTQSPAT LSLSPGERAT LSCRASQSVS SYLAWYQQKP GQAPRLLIYD ASNRATGIPA RFSGSGSGTD
FTLTISSLEP EDFAVYYCQQ SSNWPRTFGQ GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGECGGGGSG GGGSGGGGSA PASSSTKKTQ LQLEHLLLDL QMILNGINNY KNPKLTSMLT AKFAMPKKAT
ELKHLQCLEE ALKPLEEVLN LAQSKNFHLR PRDLISXₐₐ₈₈ INVIVLELKGS ETTFMCEYAD ETATIVEFLN
RWITESXₐₐ₁₂₆ SIISTLT; wherein Xₐₐ₈₈ is amino acid selected from N and A, and Xₐₐ₁₂₆
is an amino acid selected from Q, H, W or A.
SEQ ID NO: 52- trastuzumab light chain
DIQMTQSPSS LSASVGDRVT ITCRASQDVN TAVAWYQQKP GKAPKLLIYS ASFLYSGVPS RFSGSRSGTD
FTLTISSLQP EDFATYYCQQ HYTTPPTFGQ GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 53- trastuzumab heavy chain
EVQLVESGGG LVQPGGSLRL SCAASGFNIK DTYIHWVRQA PGKGLEWVAR IYPTNGYTRY ADSVKGRFTI
SADTSKNTAY LQMNSLRAED TAVYYCSRWG GDGFYAMDYW GQGTLVTVSS ASTKGPSVFP LAPSSKSTSG
GTAALGCLVK DYFPEPVTVS WNSGALTSGV HTFPAVLQSS GLYSLSSVVT VPSSSLGTQT YICNVNHKPS
NTKVDKKVEP PKSCDKTHTC PPCPAPELLG GPSVFLFPPK PKDTLMISRT PEVTCVVVDV SHEDPEVKFN
WYVDGVEVHN AKTKPREEQY NSTYRVVSVL TVLHQDWLNG KEYKCKVSNK ALPAPIEKTI SKAKGQPREP
QVYTLPPSRD ELTKNQVSLT CLVKGFYPSD IAVEWESNGQ PENNYKTTPP VLDSDGSFFL YSKLTVDKSR
WQQGNVFSCS VMHEALHNHY TQKSLSLSPGK
SEQ ID NO: 54- rituximab light chain
QIVLSQSPAI LSASPGEKVT MTCRASSSVS YIHWFQQKPG SSPKPWIYAT SNLASGVPVR FSGSGSGTSY
SLTISRVEAE DAATYYCQQW TSNPPTFGGG TKLEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 55- rituximab heavy chain
QVQLQQPGAE LVKPGASVKM SCKASGYTFT SYNMHWVKQT PGRGLEWIGA IYPGNGDTSY NQKFKGKATL
TADKSSSTAY MQLSSLTSED SAVYYCARST YYGGDWYFNV WGAGTTVTVS AASTKGPSVF PLAPSSKSTS
GGTAALGCLV KDYFPEPVTV SWNSGALTSG VHTFPAVLQS SGLYSLSSVV TVPSSSLGTQ TYICNVNHKP
SNTKVDKKAE PKSCDKTHTC PPCPAPELLG GPSVFLFPPK PKDTLMISRT PEVTCVVVDV SHEDPEVKFN
WYVDGVEVHN AKTKPREEQY NSTYRVVSVL TVLHQDWLNG KEYKCKVSNK ALPAPIEKTI SKAKGQPREP
QVYTLPPSRD ELTKNQVSLT CLVKGFYPSD IAVEWESNGQ PENNYKTTPP VLDSDGSFFL YSKLTVDKSR
WQQGNVFSCS VMHEALHNHY TQKSLSLSPG K
SEQ ID NO: 56- brentuximab light chain
DIVLTQSPAS LAVSLGQRAT ISCKASQSVD FDGDSYMNWY QQKPGQPPKV LIYAASNLES GIPARFSGSG
SGTDFTLNIH PVEEEDAATY YCQQSNEDPW TFGGGTKLEI KRTVAAPSVF IFPPSDEQLK SGTASVVCLL
NNFYPREAKV QWKVDNALQS GNSQESVTEQ DSKDSTYSLS STLTLSKADY EKHKVYACEV THQGLSSPVT
KSFNRGEC
SEQ ID NO: 57- brentuximab heavy chain
QIQLQQSGPE VVKPGASVKI SCKASGYTFT DYYITWVKQK PGQGLEWIGW IYPGSGNTKY NEKFKGKATL
TVDTSSSTAF MQLSSLTSED TAVYFCANYG NYWFAYWGQG TQVTVSAAST KGPSVFPLAP SSKSTSGGTA
ALGCLVKDYF PEPVTVSWNS GALTSGVHTF PAVLQSSGLY SLSSVVTVPS SSLGTQTYIC NVNHKPSNTK
VDKKVEPKSC DKTHTCPPCP APELLGGPSV FLFPPKPKDT LMISRTPEVT CVVVDVSHED PEVKFNWYVD
GVEVHNAKTK PREEQYNSTY RVVSVLTVLH QDWLNGKEYK CKVSNKALPA PIEKTISKAK GQPREPQVYT
LPPSRDELTK NQVSLTCLVK GFYPSDIAVE WESNGQPENN YKTTPPVLDS DGSFFLYSKL TVDKSRWQQG
NVFSCSVMHE ALHNHYTQKS LSLSPG
SEQ ID NO: 58- cetuximab light chain
DILLTQSPVI LSVSPGERVS FSCRASQSIG TNIHWYQQRT NGSPRLLIKY ASESISGIPS RFSGSGSGTD
FTLSINSVES EDIADYYCQQ NNNWPTTFGA GTKLELKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 59- cetuximab heavy chain
QVQLKQSGPG LVQPSQSLSI TCTVSGFSLT NYGVHWVRQS PGKGLEWLGV IWSGGNTDYN TPFTSRLSIN
KDNSKSQVFF KMNSLQSNDT AIYYCARALT YYDYEFAYWG QGTLVTVSAA STKGPSVFPL APSSKSTSGG
TAALGCLVKD YFPEPVTVSW NSGALTSGVH TFPAVLQSSG LYSLSSVVTV PSSSLGTQTY ICNVNHKPSN
TKVDKKVEPK SCDKTHTCPP CPAPELLGGP SVFLFPPKPK DTLMISRTPE VTCVVVDVSH EDPEVKFNWY
VDGVEVHNAK TKPREEQYNS TYRVVSVLTV LHQDWLNGKE YKCKVSNKAL PAPIEKTISK AKGQPREPQV
YTLPPSRDEL TKNQVSLTCL VKGFYPSDIA VEWESNGQPE NNYKTTPPVL DSDGSFFLYS KLTVDKSRWQ
QGNVFSCSVM HEALHNHYTQ KSLSLSPGK
SEQ ID NO: 60- panitumumab light chain
DIQMTQSPSS LSASVGDRVT ITCQASQDIS NYLNWYQQKP GKAPKLLIYD ASNLETGVPS RFSGSGSGTD
FTFTISSLQP EDIATYFCQH FDHLPLAFGG GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 61- panitumumab heavy chain
GHIYYSGNTN YNPSLKSRLT ISIDTSKTQF SLKLSSVTAA DTAIYYCVRD RVTGAFDIWG QGTMVTVSSA
STKGPSVFPL APCSRSTSES TAALGCLVKD YFPEPVTVSW NSGALTSGVH TFPAVLQSSG LYSLSSVVTV
PSSNFGTQTY TCNVDHKPSN TKVDERKCCV ECPAGPSVFL FPPKPKDTLM ISRTPEVTCV VVDVSHEDPE
VQFNWYVDGV EVHNAKTKPR EEQFNSTFRV VSVLTVVHQD WLNGKEYKCK VSNKGLPAPI EKTISKTKGQ
PREPQVYTLP PSREEMTKNQ VSLTCLVKGF YPSDIAVEWE SNGQPENNYK TTPPMLDSDG SFFLYSKLTV
DKSRWQQGNV FSCSVMHEAL HNHYTQKSLS LSPGK
SEQ ID NO: 62- anti-c-MET antibody light chain
DIVMTQAAPS VPVTPGESVS ISCRSSKSLL HSNGNTYLYW FLQRPGQSPQ VLIYRMSNLA SGVPDRFSGS
GSGTAFTLRI RRVEAEDVGV YYCMQNLEYP FTFGGGTKLE IKRTVAAPSV FIFPPSDEQL KSGTASVVCL
LNNFYPREAK VQWKVDNALQ SGNSQESVTE QDSKDSTYSL SSTLTLSKAD YEKHKVYACE VTHQGLSSPV
TKSFNRGEC
SEQ ID NO: 63- anti-c-MET antibody heavy chain
QVQLQQSGPE LVKSGASVKM SCKASGNTLK DDHVHWVKQR PGQGLEWIGW IYPGGGRTRY NEKFKGKTTL
TADKPSSTVN MLLSSLTSED SAIYFCTNLV FDVWGAGTTV TVSSASTKGP SVFPLAPSSK STSGGTAALG
CLVKDYFPEP VTVSWNSGAL TSGVHTFPAV LQSSGLYSLS SVVTVPSSSL GTQTYICNVN HKPSNTKVDK
KVEPKSCDKT HTCPPCPAPE LLGGPSVFLF PPKPKDTLMI SRTPEVTCVV VDVSHEDPEV KFNWYVDGVE
VHNAKTKPRE EQYNSTYRVV SVLTVLHQDW LNGKEYKCKV SNKALPAPIE KTISKAKGQP REPQVYTLPP
SREEMTKNQV SLTCLVKGFY PSDIAVEWES NGQPENNYKT TPPVLDSDGS FFLYSKLTVD KSRWQQGNVF
SCSVMHEALH NHYTQKSLSL SPGK
SEQ ID NO: 64- anti-GPC3 antibody light chain
DVVMTQSPLS LPVTPGEPAS ISCRSSQSLV HSNANTYLHW YLQKPGQSPQ LLIYKVSNRF SGVPDRFSGS
GSGTDFTLKI SRVEAEDVGV YYCSQNTHVP PTFGQGTKLE IKRTVAAPSV FIFPPSDEQL KSGTASVVCL
LNNFYPREAK VQWKVDNALQ SGNSQESVTE QDSKDSTYSL SSTLTLSKAD YEKHKVYACE VTHQGLSSPV
TKSFNRGEC
SEQ ID NO: 65- anti-GPC3 antibody heavy chain
QVQLVQSGAE VKKPGASVKV SCKASGYTFT DYEMHWVRQA PGQGLEWMGA LDPKTGDTAY SQKFKGRVTL
TADKSTSTAY MELSSLTSED TAVYYCTRFY SYTYWGQGTL VTVSSASTKG PSVFPLAPSS KSTSGGTAAL
GCLVKDYFPE PVTVSWNSGA LTSGVHTFPA VLQSSGLYSL SSVVTVPSSS LGTQTYICNV NHKPSNTKVD
KKVEPKSCDK THTCPPCPAP ELLGGPSVFL FPPKPKDTLM ISRTPEVTCV VVDVSHEDPE VKFNWYVDGV
EVHNAKTKPR EEQYNSTYRV VSVLTVLHQD WLNGKEYKCK VSNKALPAPI EKTISKAKGQ PREPQVYTLP
PSREEMTKNQ VSLTCLVKGF YPSDIAVEWE SNGQPENNYK TTPPVLDSDG SFFLYSKLTV DKSRWQQGNV
FSCSVMHEAL HNHYTQKSLS LSPGK
SEQ ID NO: 66- anti-Claudin 18.2 antibody light chain
DIVMTQSPSS LTVTAGEKVT MSCKSSQSLL NSGNQKNYLT WYQQKPGQPP KLLIYWASTR ESGVPDRFTG
SGSGTDFTLT ISSVQAEDLA VYYCQNDYSY PFTFGSGTKL EIKRTVAAPS VFIFPPSDEQ LKSGTASVVC
LLNNFYPREA KVQWKVDNAL QSGNSQESVT EQDSKDSTYS LSSTLTLSKA DYEKHKVYAC EVTHQGLSSP
VTKSFNRGEC
SEQ ID NO: 67- anti-Claudin 18.2 antibody heavy chain
QVQLQQPGAE LVRPGASVKL SCKASGYTFT SYWINWVKQR PGQGLEWIGN IYPSDSYTNY NQKFKDKATL
TVDKSSSTAY MQLSSPTSED SAVYYCTRSW RGNSFDYWGQ GTTLTVSSAS TKGPSVFPLA PSSKSTSGGT
AALGCLVKDY FPEPVTVSWN SGALTSGVHT FPAVLQSSGL YSLSSVVTVP SSSLGTQTYI CNVNHKPSNT
KVDKKVEPKS CDKTHTCPPC PAPELLGGPS VFLFPPKPKD TLMISRTPEV TCVVVDVSHE DPEVKFNWYV
DGVEVHNAKT KPREEQYNST YRVVSVLTVL HQDWLNGKEY KCKVSNKALP APIEKTISKA KGQPREPQVY
TLPPSRDELT KNQVSLTCLV KGFYPSDIAV EWESNGQPEN NYKTTPPVLD SDGSFFLYSK LTVDKSRWQQ
GNVFSCSVMH EALHNHYTQK SLSLSPGK
SEQ ID NO: 68- anti-Trop-2 antibody light chain CDR1
KASQDVSIAV A
SEQ ID NO: 69- anti-Trop-2 antibody light chain CDR2
SAS YRYT
SEQ ID NO: 70- anti-Trop-2 antibody light chain CDR3
QQHYITPLT
SEQ ID NO: 71- anti-Trop-2 antibody heavy chain CDR1
NYGMN
SEQ ID NO: 72- anti-Trop-2 antibody heavy chain CDR2
WINTYTGEPT YTDDFKG
SEQ ID NO: 73- anti-Trop-2 antibody heavy chain CDR3
GGFGSSYWYF DV
SEQ ID NO: 74- anti-mesothelin antibody light chain CDR1
SASS SVSYMH
SEQ ID NO: 75- anti-mesothelin antibody light chain CDR2
DTSKLAS
SEQ ID NO: 76- anti-mesothelin antibody light chain CDR3
QQWSGYPLT
SEQ ID NO: 77- anti-mesothelin antibody heavy chain CDR1
GYTMN
SEQ ID NO: 78- anti-mesothelin antibody heavy chain CDR2
LITPYNGASS YNQKFRG
SEQ ID NO: 79- anti-mesothelin antibody heavy chain CDR3
GGYDGRGFDY
SEQ ID NO: 80- Anti-FAP version 1 LC (protein sequence)

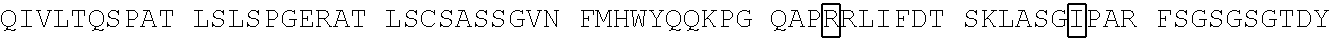

TLTISSLEPE DFAVYYCQQW SFNPPTFGQG TKVEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 81- Anti-FAP LC version 2 (protein sequence)
QIVLTQSPAT LSLSPGERAT LSCSASSGVN FMHWYQQKPG QAPKRLIFDT SKLASGVPAR FSGSGSGTDY
TLTISSLEPE DFAVYYCQQW SFNPPTFGQG TKVEIKRTVA APSVFIFPPS DEQLKSGTAS VVCLLNNFYP
REAKVQWKVD NALQSGNSQE SVTEQDSKDS TYSLSSTLTL SKADYEKHKV YACEVTHQGL SSPVTKSFNR
GEC
SEQ ID NO: 82- Anti-FAP VH (protein sequence)
QVQLVQSGAE VKKPGASVKV SCKASGYTFT NNGINWLRQA PGQGLEWMGE IYPRSTNTLY AQKFQGRVTI
TADRSSNTAY MELSSLRSED TAVYFCARTL TAPFAFWGQG TLVTVSSAST KGPSVFPLAP SSKSTSGGTA
ALGCLVKDYF PEPVTVSWNS GALTSGVHTF PAVLQSSGLY SLSSVVTVPS SSLGTQTYIC NVNHKPSNTK
VDKKVEPKSC DKTHTCPPCP APELLGGPSV FLFPPKPKDT LMISRTPEVT CVVVDVSHED PEVKFNWYVD
GVEVHNAKTK PREEQYNSTY RVVSVLTVLH QDWLNGKEYK CKVSNKALPA PIEKTISKAK GQPREPQVYT
LPPSRDELTK NQVSLTCLVK GFYPSDIAVE WESNGQPENN YKTTPPVLDS DGSFFLYSKL TVDKSRWQQG
NVFSCSVMHE ALHNHYTQKS LSLSPGK
SEQ ID NO: 83- Humanized Light Chain variable domain of FAPalpha antibody
BIBH1
DIVMTQSPDS LAVSLGERAT INCKSSQSLL YSRNQKNYLA WYQQKPGQPP KLLIFWASTR ESGVPDRFSG
SGFGTDFTLT ISSLQAEDVA VYYCQQYFSY PLTFGQGTKV EIK
SEQ ID NO: 84- Humanized Heavy Chain variable domain FAPalpha antibody BIBH1
QVQLVQSGAE VKKPGASVK VSCKTSRYTFT EYTIHWVRQA PGQRLEWIGG INPNNGIPNY NQKFKGRVTI
TVDTSASTAY MELSSLRSED TAVYYCARRR IAYGYDEGHA MDYWGQGTLV
TVSS
SEQ ID NO: 85- Humanized H8 anti-5T4 version 1 VH (protein sequence)
QVQLVQSGAE VKKPGASVKV SCKASGYSFT GYYMHWVKQS PGQGLEWIGR INPNNGVTLY NQKFKDRVTM
TRDTSISTAY MELSRLRSDD TAVYYCARST MITNYVMDYW GQGTLWTVSS
SEQ ID NO: 86- Humanized H8 anti-5T4 VH version 2 (protein sequence)
QVQLVQSGAE VKKPGASVKV SCKASGYSFT GYYMHWVRQA PGQGLEWMGR INPNNGVTLY NQKFKDRVTM
TRDTSISTAY MELSRLRSDD TAVYYCARST MITNYVMDYW GQGTLVTVSS
SEQ ID NO: 87- Humanized H8 anti-5T4 version 1 VL (protein sequence)
DIVMTQSPDS LAVSLGERAT INCKASQSVS NDVAWYQQKP GQSPKLLISY TSSRYAGVPD RFSGSGSGTD
FTLTISSLQA EDVAVYFCQQ DYNSPPTFGG GTKLEIK
SEQ ID NO: 88- Humanized H8 anti-5T4 VL version 2 (protein sequence)
DIVMTQSPDS LAVSLGERAT INCKASQSVS NDVAWYQQKP GQPPKLLIYY TSSRYAGVPD RFSGSGSGTD
FTLTISSLQA EDVAVYYCQQ DYNSPPTFGG GTKLEIK
SEQ ID NO: 89- Anti-PDL1 atezolizumab LC
DIQMTQSPSS LSASVGDRVT ITCRASQDVS TAVAWYQQKP GKAPKLLIYS ASFLYSGVPS RFSGSGSGTD
FTLTISSLQP EDFATYYCQQ YLYHPATFGQ GTKVEIKRTV AAPSVFIFPP SDEQLKSGTA SVVCLLNNFY
PREAKVQWKV DNALQSGNSQ ESVTEQDSKD STYSLSSTLT LSKADYEKHK VYACEVTHQG LSSPVTKSFN
RGEC
SEQ ID NO: 90- Anti-PDL1 atezolizumab HC (protein sequence)
EVQLVESGGG LVQPGGSLRL SCAASGFTFS DSWIHWVRQA PGKGLEWVAW ISPYGGSTYY ADSVKGRFTI
SADTSKNTAY LQMNSLRAED TAVYYCARRH WPGGFDYWGQ GTLVTVSSAS TKGPSVFPLA PSSKSTSGGT
AALGCLVKDY FPEPVTVSWN SGALTSGVHT FPAVLQSSGL YSLSSVVTVP SSSLGTQTYI CNVNHKPSNT
KVDKKVEPKS CDKTHTCPPC PAPELLGGPS VFLFPPKPKD TLMISRTPEV TCVVVDVSHE DPEVKFNWYV
DGVEVHNAKT KPREEQYAST YRVVSVLTVL HQDWLNGKEY KCKVSNKALP APIEKTISKA KGQPREPQVY
TLPPSREEMT KNQVSLTCLV KGFYPSDIAV EWESNGQPEN NYKTTPPVLD SDGSFFLYSK LTVDKSRWQQ
GNVFSCSVMH EALHNHYTQK SLSLSPGK
SEQ ID NO: 91- Anti-PD-1 Nivolumab HC (protein sequence)
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

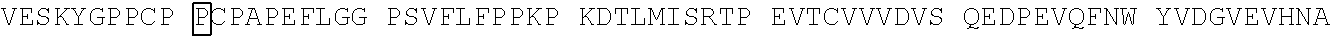

KTKPREEQFN STYRVVSVLT VLHQDWLNGK EYKCKVSNKG LPSSIEKTIS KAKGQPREPQ VYTLPPSQEE
MTKNQVSLTC LVKGFYPSDI AVEWESNGQP ENNYKTTPPV LDSDGSFFLY SRLTVDKSRW QEGNVFSCSV
MHEALHNHYT QKSLSLSLGK
SEQ ID NO: 92- Anti-PD-1 Pembrolizumab LC (protein sequence)
EIVLTQSPAT LSLSPGERAT LSCRASKGVS TSGYSYLHWY QQKPGQAPRL LIYLASYLES GVPARFSGSG
SGTDFTLTIS SLEPEDFAVY YCQHSRDLPL TFGGGTKVEI KRTVAAPSVF IFPPSDEQLK SGTASVVCLL
NNFYPREAKV QWKVDNALQS GNSQESVTEQ DSKDSTYSLS STLTLSKADY EKHKVYACEV THQGLSSPVT
KSFNRGEC
SEQ ID NO: 93- Anti-PD-1 Pembrolizumab HC (protein sequence)
QVQLVQSGVE VKKPGASVKV SCKASGYTFT NYYMYWVRQA PGQGLEWMGG INPSNGGTNF NEKFKNRVTL
TTDSSTTTAY MELKSLQFDD TAVYYCARRD YRFDMGFDYW GQGTTVTVSS ASTKGPSVFP LAPCSRSTSE
STAALGCLVK DYFPEPVTVS WNSGALTSGV HTFPAVLQSS GLYSLSSVVT VPSSSLGTKT YTCNVDHKPS

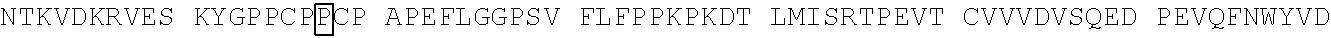

GVEVHNAKTK PREEQFNSTY RVVSVLTVLH QDWLNGKEYK CKVSNKGLPS SIEKTISKAK GQPREPQVYT
LPPSQEEMTK NQVSLTCLVK GFYPSDIAVE WESNGQPENN YKTTPPVLDS DGSFFLYSRL TVDKSRWQEG
NVFSCSVMHE ALHNHYTQKS LSLSLGK
SEQ ID NO: 94- Fc- IL-21 with knobs mutations
DKTHTCPPCP APELLGGPSV FLFPPKPKDT LMISRTPEVT CVVVDVSHED PEVKFNWYVD GVEVHNAKTK

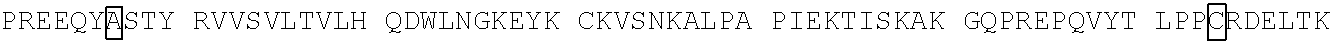

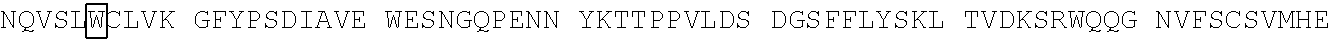

ALHNHYTQKS LSLSPGAGGG GSGGGGSGGG GSQGQDRHMI RMRQLIDIVD QLKNYVNDLV PEFLPAPEDV
ETNCEWSAFS CFQKAQLKSA NTGNNERIIN VSIKKLKRKP PSTNAGRRQK HRLTCPSCDS YEKKPPKEFL
ERFKSLLQKM IHQHLSSRTH GSEDS
SEQ ID NO: 95- Fc knob- IL-21 mutein Q19K/E109R
DKTHTCPPCP APELLGGPSV FLFPPKPKDT LMISRTPEVT CVVVDVSHED PEVKFNWYVD GVEVHNAKTK

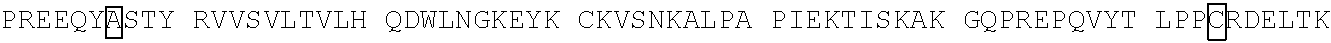

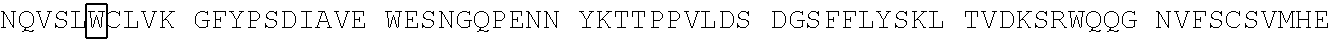

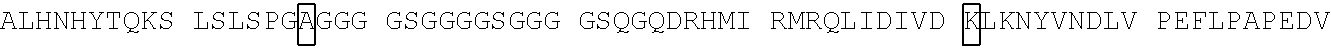

ETNCEWSAFS CFQKAQLKSA NTGNNERIIN VSIKKLKRKP PSTNAGRRQK HRLTCPSCDS YEKKPPKEFL

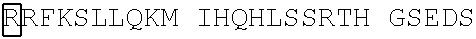

SEQ ID NO: 96- IgG1 Fc with holes mutations

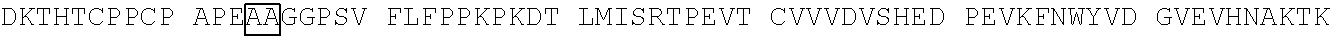

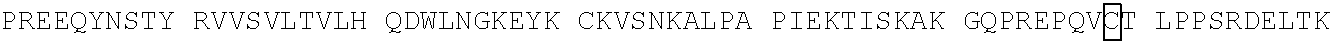

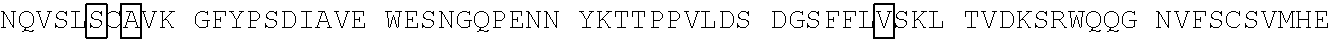

ALHNHYTQKS LSLSPGK
SEQ ID NO: 97- human anti-human IL-21 antibody clone 366.552.11.31 HC
variable domain
MKHLWFFLLL VAAPRWVLSQ VQLQESGPGL VKPSETLSLT CTVSGGSISS DFWGWIRQPP GKGLEWIGYI
SSRGSTNYNP SLKRRVTISV DTSRNQFSLK LSSVTAADTA VYYCARSAGV TDFDFWGQGT LVTVSS
SEQ ID NO: 98- human anti-human IL-21 antibody clone 366.552.11.31 LC
variable domain
MDMMVPAQLL GLLLLWFPGS RCDIQMTQSP SSVSASVGDR VTITCRASQG ISSWLAWYQH KPGKAPKLLI
YVASSLQSGV PSRFSGSGSG TDFTLTISSL QPEDFATYYC QQANSFPLTF GGGTKVEIK
SEQ ID NO: 99- human anti-human IL-21 antibody clone 362.78.1.44 HC variable
domain
MEFGLSWVFL VALLRGVQCQ VQLVESGGGV VQPGRSLRLS CAASGFTFSS YGMHWVRQAP GKGLEWVAFI
WYDGSDKYYA DSVKGRFTIS RDNSKNTLYL QMNSLRAEDT AVYYCARDGD SSDWYGDYYF GMDVWGQGTT
VTVSS
SEQ ID NO: 100- human anti-human IL-21 antibody clone 362.78.1.44 LC
variable domain
METPAQLLFL LLLWLPDTTG EIVLTQSPGT LSLSPGERAT LSCRASQSVS SSYLAWYQQK PGQAPRLLIY
GASSRATGIP DRFSGSGSGT DFTLTISRLE PEDFAVYYCQ QYGSWTFGQG TKVEIK
SEQ ID NO: 101- Fc- scFv-VH/VL 366 with holes mutations

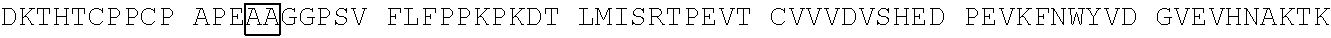

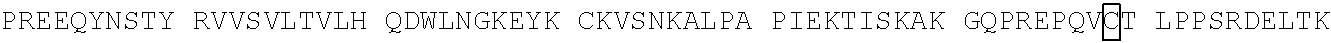

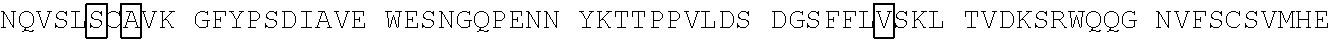

ALHNHYTQKS LSLSPGAGGG GSGGGGSGPL GVRGGGGSQV QLQESGPGLV KPSETLSLTC TVSGGSISSD
FWGWIRQPPG KGLEWIGYIS SRGSTNYNPS LKRRVTISVD TSRNQFSLKL SSVTAADTAV YYCARSAGVT
DFDFWGQGTL VTVSS (GGGGS)ₙDI QMTQSPSSVS ASVGDRVTIT CRASQGISSW LAWYQHKPGK
APKLLIYVAS SLQSGVPSRF SGSGSGTDFT LTISSLQPED FATYYCQQAN SFPLTFGGGT KVEIK;
wherein n = 1, 2, 3, 4, or 5.
SEQ ID NO: 102- Fc- scFv-VL/VH 366 with holes mutations

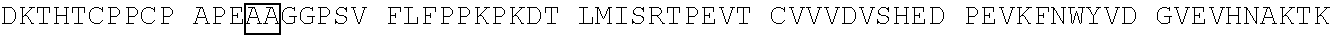

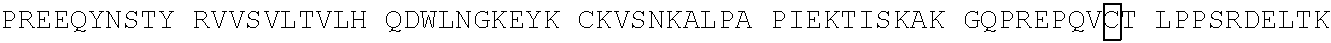

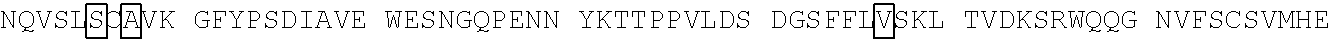

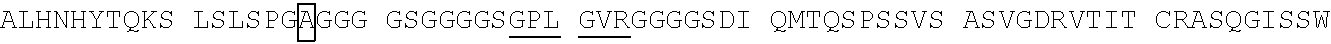

LAWYQHKPGK APKLLIYVAS SLQSGVPSRF SGSGSGTDFT LTISSLQPED FATYYCQQAN SFPLTFGGGT
KVEIK (GGGGS)ₙQV QLQESGPGLV KPSETLSLTC TVSGGSISSD FWGWIRQPPG KGLEWIGYIS
SRGSTNYNPS LKRRVTISVD TSRNQFSLKL SSVTAADTAV YYCARSAGVT DFDFWGQGTL VTVSS;
wherein n = 1, 2, 3, 4, or 5.
SEQ ID NO: 103- Fc- scFv-VH/VL 362 with holes mutations

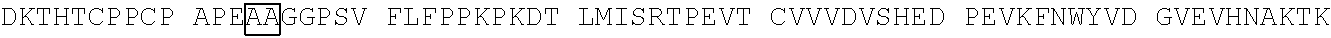

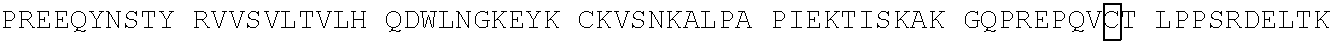

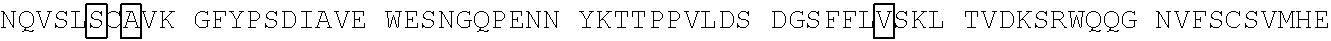

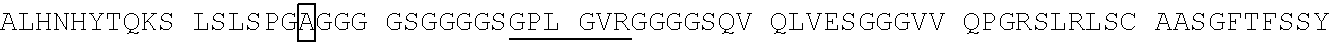

GMHWVRQAPG KGLEWVAFIW YDGSDKYYAD SVKGRFTISR DNSKNTLYLQ MNSLRAEDTA VYYCARDGDS
SDWYGDYYFG MDVWGQGTTV TVSS (GGGGS)ₙEI VLTQSPGTLS LSPGERATLS CRASQSVSSS
YLAWYQQKPG QAPRLLIYGA SSRATGIPDR FSGSGSGTDF TLTISRLEPE DFAVYYCQQY GSWTFGQGTK
VEIK; wherein n = 1, 2, 3, 4, or 5.
SEQ ID NO: 104- Fc- scFv-VL/VH 362 with holes mutations

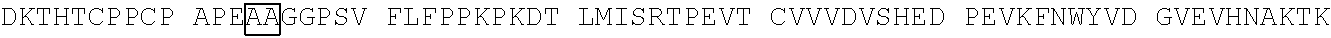

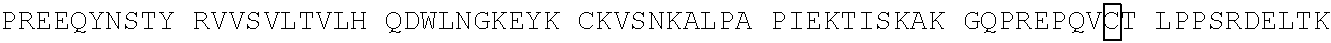

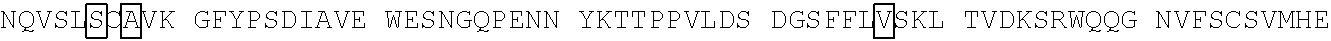

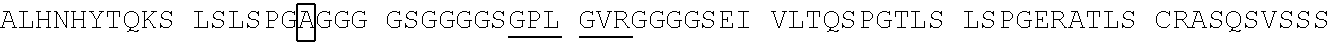

YLAWYQQKPG QAPRLLIYGA SSRATGIPDR FSGSGSGTDF TLTISRLEPE DFAVYYCQQY GSWTFGQGTK
VEIK (GGGGS)ₙQV QLVESGGGVV QPGRSLRLSC AASGFTFSSY GMHWVRQAPG KGLEWVAFIW
YDGSDKYYAD SVKGRFTISR DNSKNTLYLQ MNSLRAEDTA VYYCARDGDS SDWYGDYYFG MDVWGQGTTV
TVSS; wherein n = 1, 2, 3, 4, or 5.
SEQ ID NO: 105- scFv-Fc VH/VL 366 with holes mutations
QVQLQESGPG LVKPSETLSL TCTVSGGSIS SDFWGWIRQP PGKGLEWIGY ISSRGSTNYN PSLKRRVTIS
VDTSRNQFSL KLSSVTAADT AVYYCARSAG VTDFDFWGQG TLVTVSS (GGGGS)ₙDI QMTQSPSSVS
ASVGDRVTIT CRASQGISSW LAWYQHKPGK APKLLIYVAS SLQSGVPSRF SGSGSGTDFT LTISSLQPED

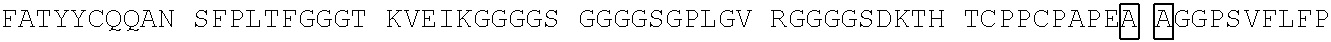

PKPKDTLMIS RTPEVTCVVV DVSHEDPEVK FNWYVDGVEV HNAKTKPREE QYNSTYRVVS VLTVLHQDWL

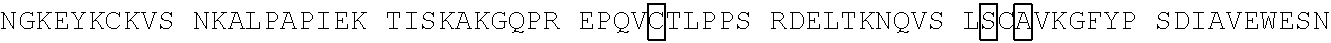

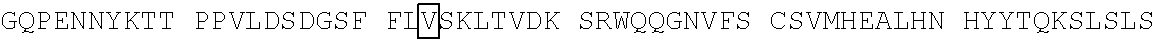

PGK; wherein n = 1, 2, 3, 4, or 5.
SEQ ID NO: 106- scFv-Fc VL/VH 366 with holes mutations
DIQMTQSPSS VSASVGDRVT ITCRASQGIS SWLAWYQHKP GKAPKLLIYV ASSLQSGVPS RFSGSGSGTD
FTLTISSLQP EDFATYYCQQ ANSFPLTFGG GTKVEIK (GGGGS)ₙQV QLQESGPGLV KPSETLSLTC
TVSGGSISSD FWGWIRQPPG KGLEWIGYIS SRGSTNYNPS LKRRVTISVD TSRNQFSLKL SSVTAADTAV

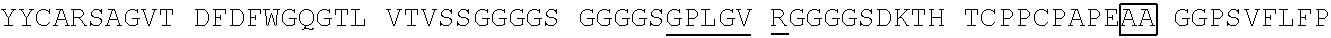

PKPKDTLMIS RTPEVTCVVV DVSHEDPEVK FNWYVDGVEV HNAKTKPREE QYNSTYRVVS VLTVLHQDWL

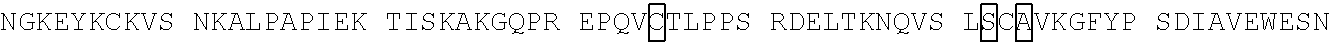

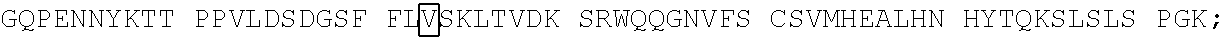

wherein n = 1, 2, 3, 4, or 5.
SEQ ID NO: 107- scFv-Fc VH/VL 362 with holes mutations
QVQLVESGGG VVQPGRSLRL SCAASGFTFS SYGMHWVRQA PGKGLEWVAF IWYDGSDKYY ADSVKGRFTI
SRDNSKNTLY LQMNSLRAED TAVYYCARDG DSSDWYGDYY FGMDVWGQGT TVTVSS (GGGGS)ₙEI
VLTQSPGTLS LSPGERATLS CRASQSVSSS YLAWYQQKPG QAPRLLIYGA SSRATGIPDR FSGSGSGTDF

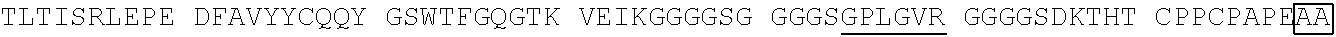

GGPSVFLFPP KPKDTLMISR TPEVTCVVVD VSHEDPEVKF NWYVDGVEVH NAKTKPREEQ YNSTYRVVSV

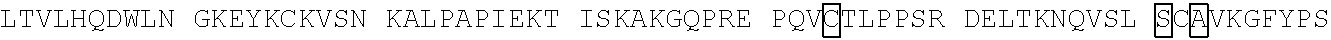

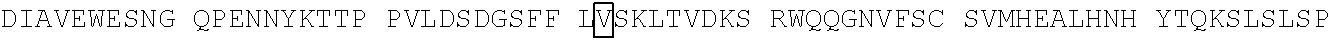

GK; wherein n = 1, 2, 3, 4, or 5.
SEQ ID NO: 108- scFv- Fc VL/VH 362 with holes mutations
EIVLTQSPGT LSLSPGERAT LSCRASQSVS SSYLAWYQQK PGQAPRLLIY GASSRATGIP DRFSGSGSGT
DFTLTISRLE PEDFAVYYCQ QYGSWTFGQG TKVEIK (GGGGS)ₙQV QLVESGGGVV QPGRSLRLSC
AASGFTFSSY GMHWVRQAPG KGLEWVAFIW YDGSDKYYAD SVKGRFTISR DNSKNTLYLQ MNSLRAEDTA

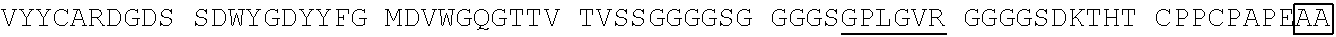

GGPSVFLFPP KPKDTLMISR TPEVTCVVVD VSHEDPEVKF NWYVDGVEVH NAKTKPREEQ YNSTYRVVSV

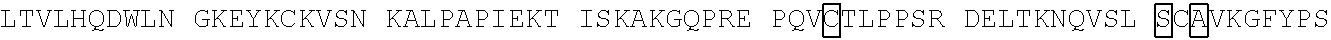

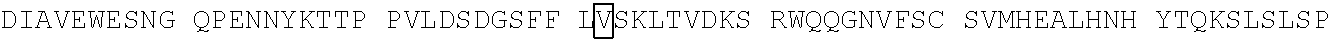

GK; wherein n = 1, 2, 3, 4, or 5.
SEQ ID NO: 109- PD1-HC- scFv-VH/VL 366 with holes mutations
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

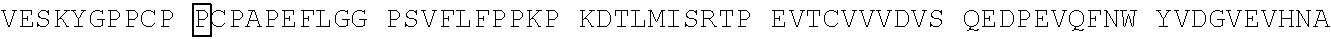

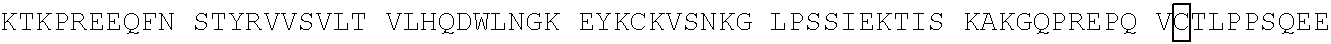

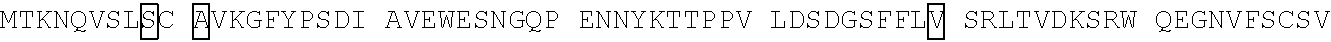

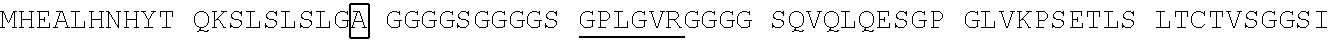

SSDFWGWIRQ PPGKGLEWIG YISSRGSTNY NPSLKRRVTI SVDTSRNQFS LKLSSVTAAD TAVYYCARSA
GVTDFDFWGQ GTLVTVSS (GGGGS)ₙDI QMTQSPSSVS ASVGDRVTIT CRASQGISSW LAWYQHKPGK
APKLLIYVAS SLQSGVPSRF SGSGSGTDFT LTISSLQPED FATYYCQQAN SFPLTFGGGT KVEIK;
wherein n = 1, 2, 3, 4, or 5.
SEQ ID NO: 110- PD1-HC- scFv-VL/VH 366 with holes mutations
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

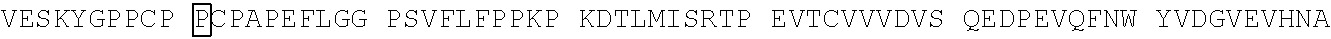

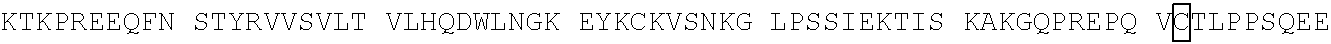

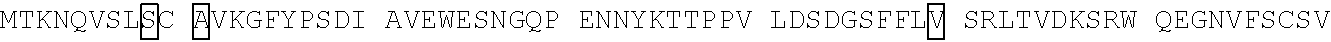

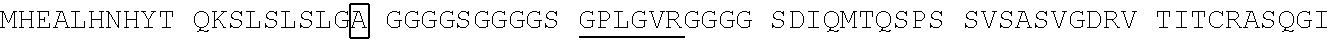

SSWLAWYQHK PGKAPKLLIY VASSLQSGVP SRFSGSGSGT DFTLTISSLQ PEDFATYYCQ QANSFPLTFG
GGTKVEIK (GGGGS)ₙQV QLQESGPGLV KPSETLSLTC TVSGGSISSD FWGWIRQPPG KGLEWIGYIS
SRGSTNYNPS LKRRVTISVD TSRNQFSLKL SSVTAADTAV YYCARSAGVT DFDFWGQGTL VTVSS;
wherein n = 1, 2, 3, 4, or 5.
SEQ ID NO: 111-PD1-HC-scFv-VH/VL362withholesmutations
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

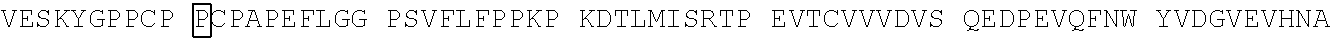

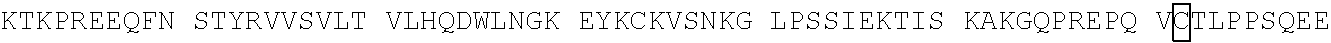

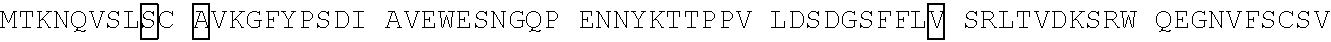

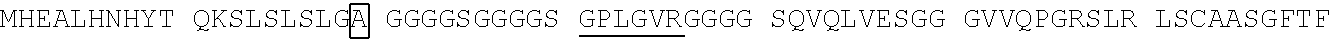

SSYGMHWVRQ APGKGLEWVA FIWYDGSDKY YADSVKGRFT ISRDNSKNTL YLQMNSLRAE DTAVYYCARD
GDSSDWYGDY YFGMDVWGQG TTVTVSS (GGGGS)ₙEI VLTQSPGTLS LSPGERATLS CRASQSVSSS
YLAWYQQKPG QAPRLLIYGA SSRATGIPDR FSGSGSGTDF TLTISRLEPE DFAVYYCQQY GSWTFGQGTK
VEIK; wherein n = 1, 2, 3, 4, or 5.
SEQ ID NO: 112- PD1-HC- scFv-VL/VH 362 with holes mutations
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

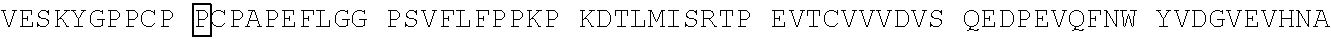

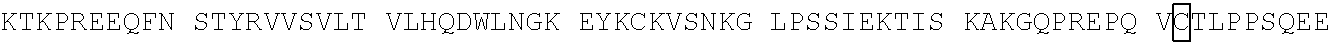

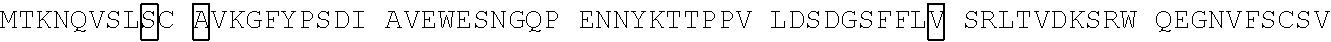

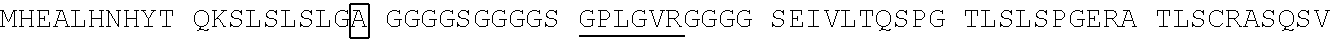

SSSYLAWYQQ KPGQAPRLLI YGASSRATGI PDRFSGSGSG TDFTLTISRL EPEDFAVYYC QQYGSWTFGQ
GTKVEIK (GGGGS)ₙQV QLVESGGGVV QPGRSLRLSC AASGFTFSSY GMHWVRQAPG KGLEWVAFIW
YDGSDKYYAD SVKGRFTISR DNSKNTLYLQ MNSLRAEDTA VYYCARDGDS SDWYGDYYFG MDVWGQGTTV
TVSS; wherein n = 1, 2, 3, 4, or 5.
SEQ ID NO: 113- IL-2Rβ ECD- Fc- scFv-VH/VL 366 with holes mutations
AVNGTSQFTC FYNSRANISC VWSQDGALQD TSCQVHAWPD RRRWNQTCEL LPVSQASWAC NLILGAPDSQ
KLTTVDIVTL RVLCREGVRW RVMAIQDFKP FENLRLMAPI SLQVVHVETH RCNISWEISQ ASHYFERHLE
FEARTLSPGH TWEEAPLLTL KQKQEWICLE TLTPDTQYEF QVRVKPLQGE FTTWSPWSQP LAFRTKPAAL

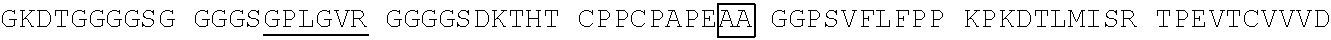

VSHEDPEVKF NWYVDGVEVH NAKTKPREEQ YNSTYRVVSV LTVLHQDWLN GKEYKCKVSN KALPAPIEKT

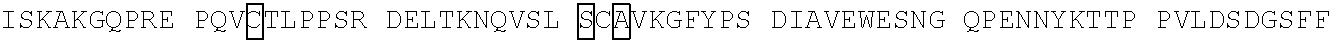

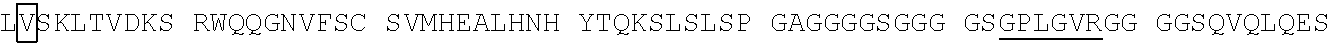

GPGLVKPSET LSLTCTVSGG SISSDFWGWI RQPPGKGLEW IGYISSRGST NYNPSLKRRV TISVDTSRNQ
FSLKLSSVTA ADTAVYYCAR SAGVTDFDFW GQGTLVTVSS (GGGGS)ₙDI QMTQSPSSVS ASVGDRVTIT
CRASQGISSW LAWYQHKPGK APKLLIYVAS SLQSGVPSRF SGSGSGTDFT LTISSLQPED FATYYCQQAN
SFPLTFGGGT KVEIK; wherein n = 1, 2, 3, 4, or 5.
SEQ ID NO: 114- IL-2Rβ ECD- Fc- scFv-VL/VH 366 with holes mutations
AVNGTSQFTC FYNSRANISC VWSQDGALQD TSCQVHAWPD RRRWNQTCEL LPVSQASWAC NLILGAPDSQ
KLTTVDIVTL RVLCREGVRW RVMAIQDFKP FENLRLMAPI SLQVVHVETH RCNISWEISQ ASHYFERHLE
FEARTLSPGH TWEEAPLLTL KQKQEWICLE TLTPDTQYEF QVRVKPLQGE FTTWSPWSQP LAFRTKPAAL

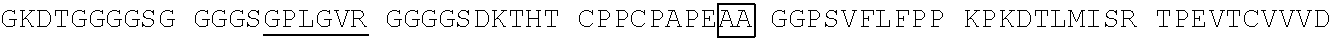

VSHEDPEVKF NWYVDGVEVH NAKTKPREEQ YNSTYRVVSV LTVLHQDWLN GKEYKCKVSN KALPAPIEKT

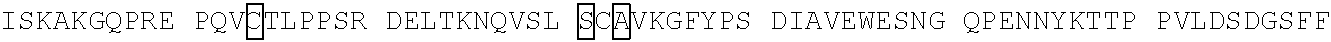

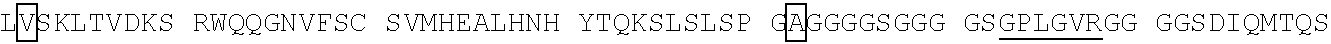

PSSVSASVGD RVTITCRASQ GISSWLAWYQ HKPGKAPKLL IYVASSLQSG VPSRFSGSGS GTDFTLTISS
LQPEDFATYY CQQANSFPLT FGGGTKVEIK (GGGGS)ₙQV QLQESGPGLV KPSETLSLTC TVSGGSISSD
FWGWIRQPPG KGLEWIGYIS SRGSTNYNPS LKRRVTISVD TSRNQFSLKL SSVTAADTAV YYCARSAGVT
DFDFWGQGTL VTVSS; wherein n = 1, 2, 3, 4, or 5.
SEQ ID NO: 115- IL-2Rβ ECD- Fc- scFv-VH/VL 362 with holes mutations
AVNGTSQFTC FYNSRANISC VWSQDGALQD TSCQVHAWPD RRRWNQTCEL LPVSQASWAC NLILGAPDSQ
KLTTVDIVTL RVLCREGVRW RVMAIQDFKP FENLRLMAPI SLQVVHVETH RCNISWEISQ ASHYFERHLE
FEARTLSPGH TWEEAPLLTL KQKQEWICLE TLTPDTQYEF QVRVKPLQGE FTTWSPWSQP LAFRTKPAAL

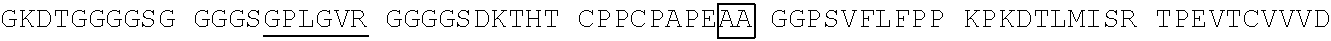

VSHEDPEVKF NWYVDGVEVH NAKTKPREEQ YNSTYRVVSV LTVLHQDWLN GKEYKCKVSN KALPAPIEKT

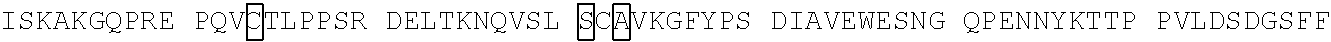

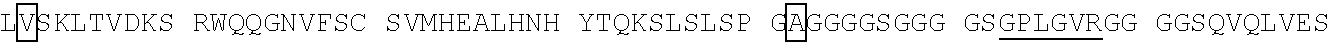

GGGVVQPGRS LRLSCAASGF TFSSYGMHWV RQAPGKGLEW VAFIWYDGSD KYYADSVKGR FTISRDNSKN
TLYLQMNSLR AEDTAVYYCA RDGDSSDWYG DYYFGMDVWG QGTTVTVSS (GGGGS)ₙEI VLTQSPGTLS
LSPGERATLS CRASQSVSSS YLAWYQQKPG QAPRLLIYGA SSRATGIPDR FSGSGSGTDF TLTISRLEPE
DFAVYYCQQY GSWTFGQGTK VEIK; wherein n = 1, 2, 3, 4, or 5.
SEQ ID NO: 116- IL-2Rβ ECD IL-2Rβ ECD- Fc- scFv-VL/VH 362 with holes
mutations
AVNGTSQFTC FYNSRANISC VWSQDGALQD TSCQVHAWPD RRRWNQTCEL LPVSQASWAC NLILGAPDSQ
KLTTVDIVTL RVLCREGVRW RVMAIQDFKP FENLRLMAPI SLQVVHVETH RCNISWEISQ ASHYFERHLE
FEARTLSPGH TWEEAPLLTL KQKQEWICLE TLTPDTQYEF QVRVKPLQGE FTTWSPWSQP LAFRTKPAAL

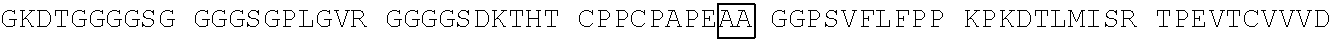

VSHEDPEVKF NWYVDGVEVH NAKTKPREEQ YNSTYRVVSV LTVLHQDWLN GKEYKCKVSN KALPAPIEKT

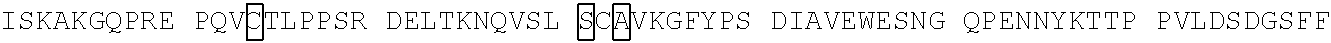

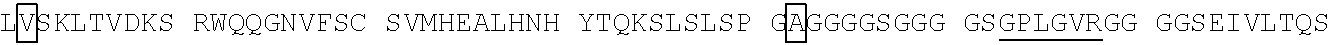

PGTLSLSPGE RATLSCRASQ SVSSSYLAWY QQKPGQAPRL LIYGASSRAT GIPDRFSGSG SGTDFTLTIS
RLEPEDFAVY YCQQYGSWTF GQGTKVEIK (GGGGS)ₙQV QLVESGGGVV QPGRSLRLSC AASGFTFSSY
GMHWVRQAPG KGLEWVAFIW YDGSDKYYAD SVKGRFTISR DNSKNTLYLQ MNSLRAEDTA VYYCARDGDS
SDWYGDYYFG MDVWGQGTTV TVSS; wherein n = 1, 2, 3, 4, or 5.
SEQ ID NO: 117 PD1-HC-IL21wt
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

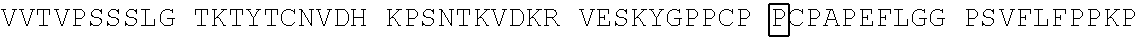

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV
MHEALHNHYT QKSLSLSLGA GGGGSGGGGS GGGGSQGQDR HMIRMRQLID IVDQLKNYVN
DLVPEFLPAP EDVETNCEWS AFSCFQKAQL KSANTGNNER IINVSIKKLK RKPPSTNAGR
RQKHRLTCPS CDSYEKKPPK EFLERFKSLL QKMIHQHLSS RTHGSEDS
SEQ ID NO:118 PD1-HC-IL21wt-cleavable-scFv
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

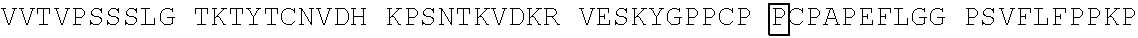

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

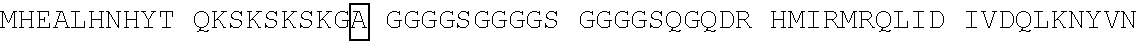

DLVPEFLPAP EDVETNCEWS AFSCFQKAQL KSANTGNNER IINVSIKKLK RKPPSTNAGR
RQKHRLTCPS CDSYEKKPPK EFLERFKSLL QKMIHQHLSS RTHGSEDS (GGGGS)ₙ₁
RQARVVNG (GGGGS)ₙ₂ EIVLTQSPGT LSLSPGERAT LSCRASQSVS SSYLAWYQQK
PGQAPRLLIY GASSRATGIP DRFSGSGSGT DFTLTISRLE PEDFAVYYCQ QYGSWTFGQG
TKVEIKGGGG SGGGGSGGGG SQVQLVESGG GVVQPGRSLR LSCAASGFTF SSYGMHWVRQ
APGKGLEWVA FIWYDGSDKY YADSVKGRFT ISRDNSKNTL YLQMNSLRAE DTAVYYCARD
GDSSDWYGDY YFGMDVWGQG TTVTVSS; wherein n1 = 1, 2, 3, 4, or 5; and n2 = 1, 2,
3, 4, or 5
SEQ ID NO: 119 PD1-HC-IL21wt-cleavable-IL21R-ECD
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

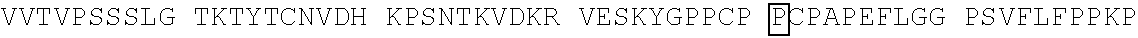

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

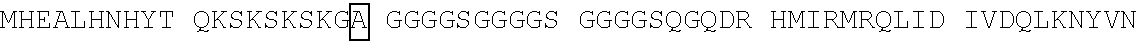

DLVPEFLPAP EDVETNCEWS AFSCFQKAQL KSANTGNNER IINVSIKKLK RKPPSTNAGR
RQKHRLTCPS CDSYEKKPPK EFLERFKSLL QKMIHQHLSS RTHGSEDS (GGGGS)ₙ₁
RQARVVNG (GGGGS)ₙ₂ CPDLVCYTDY LQTVICILEM WNLHPSTLTL TWQDQYEELK
DEATSCSLHR SAHNATHATY TCHMDVFHFM ADDIFSVNIT DQSGNYSQEC GSFLLAESIK
RAPPFNVTVT FSGQYNISWR SDYEDPAFYM LKGKLQYELQ YRNRGDPWAV SPRRKLISVD
SRSVSLLPLE FRKDSSYELQ VRAGPMPGSS YQGTWSEWSD PVIFQTQSEE LKE; wherein n1 = 1,
2, 3, 4, or 5; and n2 = 1, 2, 3, 4, or 5
SEQ ID NO: 120 PD1-HC-cleavable linker- IL21Ralpha- cleavable linker- scFv
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

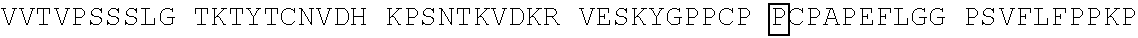

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

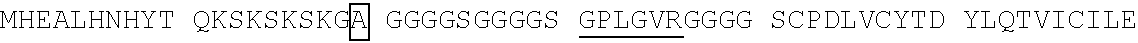

MWNLHPSTLT LTWQDQYEEL KDEATSCSLH RSAHNATHAT YTCHMDVFHF MADDIFSVNI
TDQSGNYSQE CGSFLLAESI KPAPPFNVTV TFSGQYNISW RSDYEDPAFY MLKGKLQYEL
QYRNRGDPWA VSPRRKLISV DSRSVSLLPL EFRKDSSYEL QVRAGPMPGS SYQGTWSEWS
DPVIFQTQSE ELKE(GGGGS)ₙ₁ RQARVVNG (GGGGS)ₙ₂ EIVLTQSPGT LSLSPGERAT
LSCRASQSVS SSYLAWYQQK PGQAPRLLIY GASSRATGIP DRFSGSGSGT DFTLTISRLE
PEDFAVYYCQ QYGSWTFGQG TKVEIKGGGG SGGGGSGGGG SQVQLVESGG GVVQPGRSLR
LSCAASGFTF SSYGMHWVRQ APGKGLEWVA FIWYDGSDKY YADSVKGRFT ISRDNSKNTL
YLQMNSLRAE DTAVYYCARD GDSSDWYGDY YFGMDVWGQG TTVTVSS; wherein n1 = 1, 2, 3, 4,
or 5; and n2 = 1, 2, 3, 4, or 5
SEQ ID NO: 121 PD1-HC-cleavable linker- scFv- cleavable linker- IL21Ralpha
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

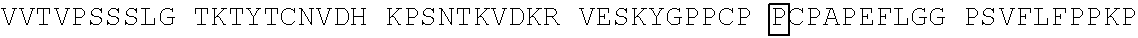

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

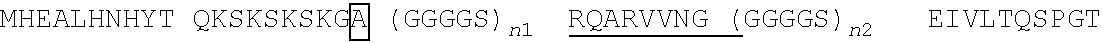

LSLSPGERAT LSCRASQSVS SSYLAWYQQK PGQAPRLLIY GASSRATGIP DRFSGSGSGT
DFTLTISRLE PEDFAVYYCQ QYGSWTFGQG TKVEIKGGGG SGGGGSGGGG SQVQLVESGG
GVVQPGRSLR LSCAASGFTF SSYGMHWVRQ APGKGLEWVA FIWYDGSDKY YADSVKGRFT
ISRDNSKNTL YLQMNSLRAE DTAVYYCARD GDSSDWYGDY YFGMDVWGQG TTVTVSS
(GGGGS)ₙ₃ GPLGVR (GGGGS)ₙ₄CP DLVCYTDYLQ TVICILEM WNLHPSTLTL
TWQDQYEELK DEATSCSLHR SAHNATHATY TCHMDVFHFM ADDIFSVNIT DQSGNYSQEC
GSFLLAESIK PAPPFNVTVT FSGQYNISWR SDYEDPAFYM LKGKLQYELQ YRNRGDPWAV
SPRRKLISVD SRSVSLLPLE FRKDSSYELQ VRAGPMPGSS YQGTWSEWSD PVIFQTQSEE
LKE; wherein n1 = 1, 2, 3, 4, or 5; n2 = 1, 2, 3, 4, or 5; n3 = 1, 2, 3, 4,
or 5; and n4 = 1, 2, 3, 4, or 5.
SEQ ID NO: 122 PD1-HC-cleavable linker- IL21Ralpha
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

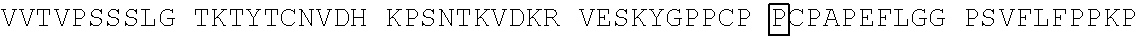

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

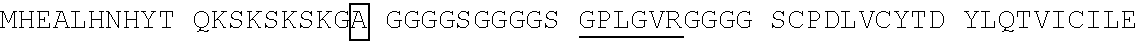

MWNLHPSTLT LTWQDQYEEL KDEATSCSLH RSAHNATHAT YTCHMDVFHF MADDIFSVNI
TDQSGNYSQE CGSFLLAESI KPAPPFNVTV TFSGQYNISW RSDYEDPAFY MLKGKLQYEL
QYRNRGDPWA VSPRRKLISV DSRSVSLLPL EFRKDSSYEL QVRAGPMPGS SYQGTWSEWS
DPVIFQTQSE ELKE
SEQ ID NO: 123 PD1-HC-cleavable linker- scFv
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

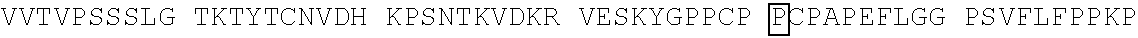

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

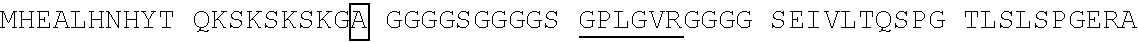

TLSCRASQSV SSSYLAWYQQ KPGQAPRLLI YGASSRATGI PDRFSGSGSG TDFTLTISRL
EPEDFAVYYC QQYGSWTFGQ GTKVEIKGGG GSGGGGSGGG GSQVQLVESG GGVVQPGRSL
RLSCAASGFT FSSYGMHWVR QAPGKGLEWV AFIWYDGSDK YYADSVKGRF TISRDNSKNT
LYLQMNSLRA EDTAVYYCAR DGDSSDWYGD YYFGMDVWGQ GTTVTVSS
SEQ ID NO: 124 PD1-HC-cleavable linker- IL21Ralpha- cleavable linker- scFv
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

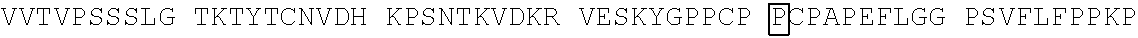

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

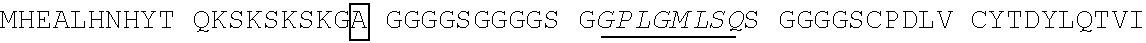

CILEMWNLHP STLTLTWQDQ YEELKDEATS CSLHRSAHNA THATYTCHMD VFHFMADDIF
SVNITDQSGN YSQECGSFLL AESIKPAPPF NVTVTFSGQY NISWRSDYED RAFYMLKGKL
QYELQYRNRG DPWAVSPRRK LISVDSRSVS LLPLEFRKDS SYELQVRAGP MPGSSYQGTW
SEWSDPVIFQ TQSEELKE (GGGGS)ₙ₁ RQARVVNG (GGGGS)ₙ₂ EIVLTQSPGT
LSLSPGERAT LSCRASQSVS SSYLAWYQQK PGQAPRLLIY GASSRATGIP DRFSGSGSGT
DFTLTISRLE PEDFAVYYCQ QYGSWTFGQG TKVEIKGGGG SGGGGSGGGG SQVQLVESGG
GVVQPGRSLR LSCAASGFTF SSYGMHWVRQ APGKGLEWVA FIWYDGSDKY YADSVKGRFT
ISRDNSKNTL YLQMNSLRAE DTAVYYCARD GDSSDWYGDY YFGMDVWGQG TTVTVSS; wherein n1 =
1, 2, 3, 4, or 5; and n2 = 1, 2, 3, 4, or 5
SEQ ID NO: 125 PD1-HC-cleavable linker- scFv- cleavable linker- IL21Ralpha
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

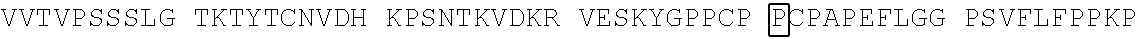

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

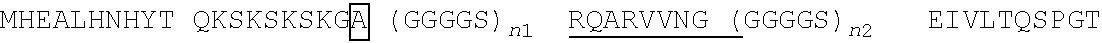

LSLSPGERAT LSCRASQSVS SSYLAWYQQK PGQAPRLLIY GASSRATGIP DRFSGSGSGT
DFTLTISRLE PEDFAVYYCQ QYGSWTFGQG TKVEIKGGGG SGGGGSGGGG SQVQLVESGG
GVVQPGRSLR LSCAASGFTF SSYGMHWVRQ APGKGLEWVA FIWYDGSDKY YADSVKGRFT
ISRDNSKNTL YLQMNSLRAE DTAVYYCARD GDSSDWYGDY YFGMDVWGQG TTVTVSS
(GGGGS)ₙ₃ GGPLGMLSQS (GGGGS)ₙ₄ CPDLVCYTDY LQTVICILEM WNLHPSTLTL
TWQDQYEELK DEATSCSLHR SAHNATHATY TCHMDVFHFM ADDIFSVNIT DQSGNYSQEC
GSFLLAESIK PAPPFNVTVT FSGQYNISWR SDYEDPAFYM LKGKLQYELQ YRNRGDPWAV
SPRRKLISVD SRSVSLLPLE FRKDSSYELQ VRAGPMPGSS YQGTWSEWSD PVIFQTQSEE
LKE; wherein n1 = 1, 2, 3, 4, or 5; n2 = 1, 2, 3, 4, or 5; n3 = 1, 2, 3, 4,
or 5; and n4 = 1, 2, 3, 4, or 5.
SEQ ID NO: 126 PD1-HC-cleavable linker- IL21Ralpha
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

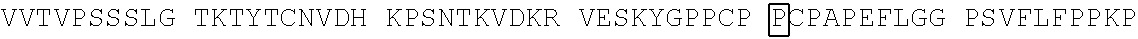

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

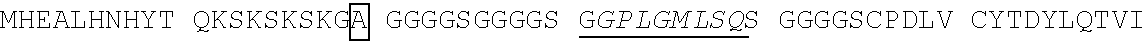

CILEMWNLHP STLTLTWQDQ YEELKDEATS CSLHRSAHNA THATYTCHMD VFHFMADDIF
SVNITDQSGN YSQECGSFLL AESIKPAPPF NVTVTFSGQY NISWRSDYED PAFYMLKGKL
QYELQYRNRG DPWAVSPRRK LISVDSRSVS LLPLEFRKDS SYELQVRAGP MPGSSYQGTW
SEWSDPVIFQ TQSEELKE
SEQ ID NO: 127 PD1-HC-cleavable linker- scEv
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

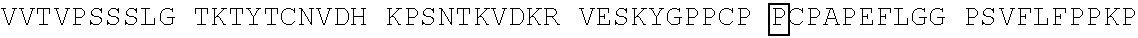

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

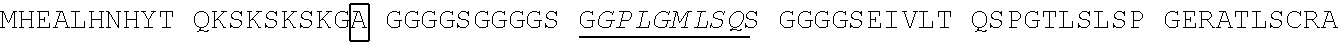

SQSVSSSYLA WYQQKPGQAP RLLIYGASSR ATGIPDRFSG SGSGTDFTLT
ISRLEPEDFA VYYCQQYGSW TFGQGTKVEI KGGGGSGGGG SGGGGSQVQL VESGGGVVQP
GRSLRLSCAA SGFTFSSYGM HWVRQAPGKG LEWVAFIWYD GSDKYYADSV KGRFTISRDN
SKNTLYLQMN SLRAEDTAVY YCARDGDSSD WYGDYYFGMD VWGQGTTVTV SS
SEQ ID NO: 128- IL-21 receptor ECD (source: uniprot.org/uniprot/Q9HBE5)
CPDLVCYTDY LQTVICILEM WNLHPSTLTL TWQDQYEELK DEATSCSLHR SAHNATHATY
TCHMDVFHFM ADDIFSVNIT DQSGNYSQEC GSFLLAESIK PAPPFNVTVT FSGQYNISWR
SDYEDPAFYM LKGKLQYELQ YRNRGDPWAV SPRRKLISVD SRSVSLLPLE FRKDSSYELQ
VRAGPMPGSS YQGTWSEWSD PVIFQTQSEE LKE
SEQ ID NO: 129 PD1-HC- IL-21 K73A
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

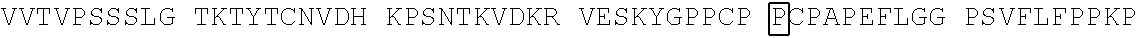

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

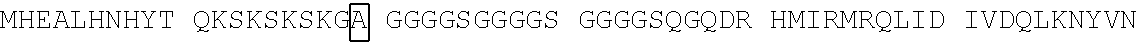

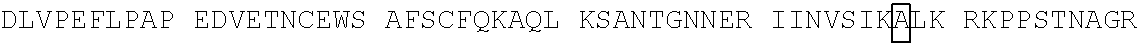

RQKHRLTCPS CDSYEKKPPK EFLERFKSLL QKMIHQHLSS RTHGSEDS
SEQ ID NO: 130- PD1 ANTIBODY-HC- non-cleavable-SCFV AGAINST IL-21
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

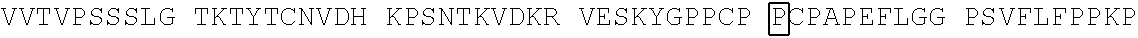

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

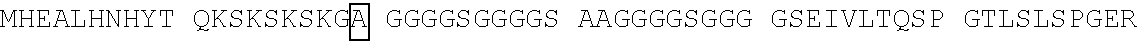

ATLSCRASQS VSSSYLAWYQ QKPGQAPRLL IYGASSRATG IPDRFSGSGS GTDFTLTISR
LEPEDFAVYY CQQYGSWTFG QGTKVEIKGG GGSGGGGSGG GGSQVQLVES GGGVVQPGRS
LRLSCAASGF TFSSYGMHWV RQAPGKGLEW VAFIWYDGSD KYYADSVKGR FTISRDNSKN
TLYLQMNSLR AEDTAVYYCA RDGDSSDWYG DYYFGMDVWG QGTTVTVSS
SEQ ID NO: 131 PD1 antibody-HC-cleavable-scFv
QVQLVESGGG VVQPGRSLRL DCKASGITFS NSGMHWVRQA PGKGLEWVAV IWYDGSKRYY
ADSVKGRFTI SRDNSKNTLF LQMNSLRAED TAVYYCATND DYWGQGTLVT VSSASTKGPS
VFPLAPCSRS TSESTAALGC LVKDYFPEPV TVSWNSGALT SGVHTFPAVL QSSGLYSLSS

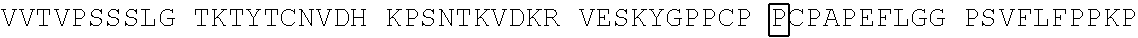

KDTLMISRTP EVTCVVVDVS QEDPEVQFNW YVDGVEVHNA KTKPREEQFN STYRVVSVLT
VLHQDWLNGK EYKCKVSNKG LPSSIEKTIS KAKGQPREPQ VYTLPPSQEE MTKNQVSLTC
LVKGFYPSDI AVEWESNGQP ENNYKTTPPV LDSDGSFFLY SRLTVDKSRW QEGNVFSCSV

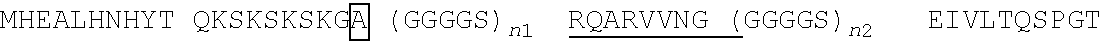

LSLSPGERAT LSCRASQSVS SSYLAWYQQK PGQAPRLLIY GASSRATGIP DRFSGSGSGT
DFTLTISRLE PEDFAVYYCQ QYGSWTFGQG TKVEIKGGGG SGGGGSGGGG SQVQLVESGG
GVVQPGRSLR LSCAASGFTF SSYGMHWVRQ APGKGLEWVA FIWYDGSDKY YADSVKGRFT
ISRDNSKNTL YLQMNSLRAE DTAVYYCARD GDSSDWYGDY YFGMDVWGQG TTVTVSS; wherein n1 =
1, 2, 3, 4, or 5; and n2 = 1, 2, 3, 4, or 5
SEQ ID NO: 132
GGGGSGGGGS AAGGGGSGGG GS
SEQ ID NO: 133 PD1 ANTIBODY HC-IL21 HOLE-SCFV-LVHV
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

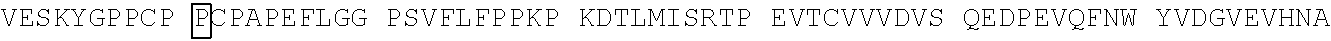

KTKPREEQFN STYRVVSVLT VLHQDWLNGK EYKCKVSNKG LPSSIEKTIS KAKGQPREPQ VCTLPPSQEE
MTKNQVSLSC AVKGFYPSDI AVEWESNGQP ENNYKTTPPV LDSDGSFFLV SRLTVDKSRW QEGNVFSCSV

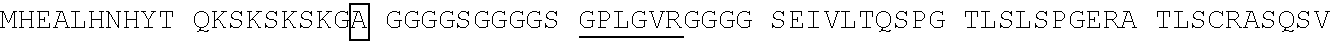

SSSYLAWYQQ KPGQAPRLLI YGASSRATGI PDRFSGSGSG TDFTLTISRL EPEDFAVYYC QQYGSWTFGQ
GTKVEIKGGG GSGGGGSGGG GSQVQLVESG GGVVQPGRSL RLSCAASGFT FSSYGMHWVR QAPGKGLEWV
AFIWYDGSDK YYADSVKGRF TISRDNSKNT LYLQMNSLRA EDTAVYYCAR DGDSSDWYGD YYFGMDVWGQ
GTTVTVSS
SEQ ID NO: 134 PD1 ANTIBODY HC HOLE
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

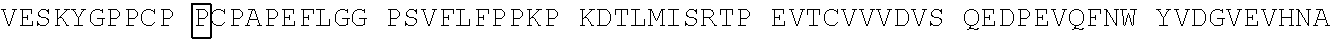

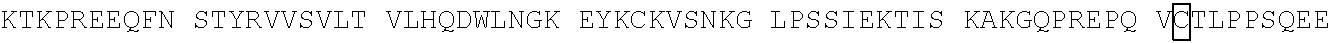

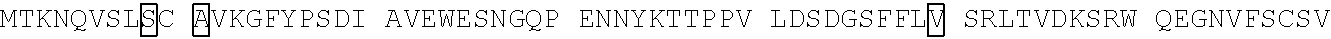

MHEALHNHYT QKSLSLSLGK
SEQ ID NO: 135 PD1 antibody HC-knob-hIL21v1(R9E, R76A)
QVQLVESGGG VVQPGRSLRL DCKASGITFS NSGMHWVRQA PGKGLEWVAV IWYDGSKRYY ADSVKGRFTI
SRDNSKNTLF LQMNSLRAED TAVYYCATND DYWGQGTLVT VSSASTKGPS VFPLAPCSRS TSESTAALGC
LVKDYFPEPV TVSWNSGALT SGVHTFPAVL QSSGLYSLSS VVTVPSSSLG TKTYTCNVDH KPSNTKVDKR

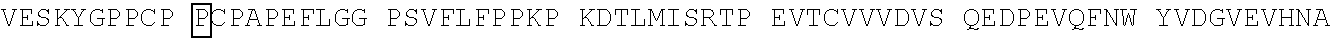

KTKPREEQFN STYRVVSVLT VLHQDWLNGK EYKCKVSNKG LPSSIEKTIS KAKGQPREPQ VYTLPPCQEE
MTKNQVSLWC LVKGFYPSDI AVEWESNGQP ENNYKTTPPV LDSDGSFFLY SRLTVDKSRW QEGNVFSCSV

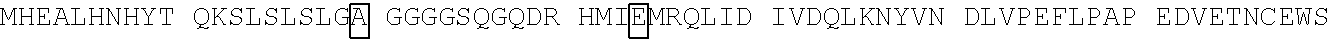

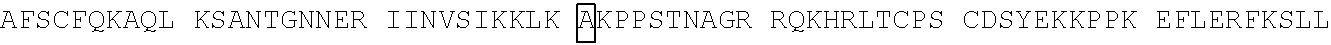

QKMIHQHLSS RTHGSEDS

1. A prodrug comprising a human IL-21 agonist polypeptide, a masking moiety, and a carrier moiety, wherein
the masking moiety comprises an antigen-binding fragment of an antibody that binds to the human IL-21 agonist polypeptide and inhibits a biological activity of the human IL-21 agonist polypeptide,
the human IL-21 agonist polypeptide is fused to the carrier moiety, and
the masking moiety is fused to the human IL-21 agonist polypeptide or to the carrier moiety, optionally through a peptide linker.

2. The prodrug of claim 1, wherein the human IL-21 agonist polypeptide comprises SEQ ID NO: 1 or an amino acid sequence that is at least 90% identical to SEQ ID NO: 1.

3. The prodrug of claim 1, wherein the IL-21 agonist polypeptide has an amino acid sequence selected from SEQ ID NO: 2, 3, 4, and 5.

4. The prodrug of any one of claims 1-3, wherein the masking moiety inhibits the binding of the IL-21 agonist polypeptide to an IL-21 receptor.

5. The prodrug of claim 4, wherein the antibody comprises a heavy chain variable domain with an amino acid sequence at least 95% identical as that of SEQ ID NO: 97 or 99, and a light chain variable domain with an amino acid sequence at least 95% identical as that of SEQ ID NO: 98 or 100.

6. The prodrug of claim 4, wherein the antigen-binding fragment of an antibody is a single chain fragment variable (scFv) comprising a heavy chain variable domain with an amino acid sequence as shown SEQ ID NO: 97 and a light chain variable domain with an amino acid sequence as shown in SEQ ID NO: 98, or a heavy chain variable domain with an amino acid sequence as shown SEQ ID NO: 99 and a light chain variable domain with an amino acid sequence as shown in SEQ ID NO: 100.

7. The prodrug of any of claims 1-6, wherein the cytokine moiety is fused to the carrier through a non-cleavable peptide linker, or the masking moiety is fused to the carrier or to the cytokine moiety through a non-cleavable peptide linker; and wherein the non-cleavable peptide linker comprises an amino acid sequence selected from SEQ ID NOs: 29-33 and 132.

8. The prodrug of any of claims 1-6, wherein the cytokine moiety is fused to the carrier through a cleavable peptide linker, or the masking moiety is fused to the carrier or to the cytokine moiety through a cleavable peptide linker.

9. The prodrug of claim 8, wherein the cleavable peptide linker comprises a substrate sequence of urokinase-type plasminogen activator (uPA), matrix metallopeptidase (MMP) 2, MMP9, or matriptase.

10. The prodrug of claim 8, wherein the cleavable peptide linker comprises substrate sequences of (i) both uPA and MMP2, (ii) both uPA and MMP9, or (iii) matriptase, MMP2 and MMP9.

11. The prodrug of claim 8, wherein the cleavable peptide linker comprises an amino acid sequence selected from SEQ ID NOs: 11-26.

12. The prodrug of any one of claims 8-11, wherein the cleavable peptide linker is cleavable by one or more proteases located at a tumor site or its surrounding environment, and the cleavage leads to activation of the prodrug at the tumor site or surrounding environment.

13. The prodrug of any one of the preceding claims, wherein the carrier moiety is an antibody Fc domain, an antibody, or an antigen-binding fragment of an antibody.

14. The prodrug of claim 13, wherein the carrier moiety is an antibody Fc domain or an antibody comprising knobs-into-holes mutations, and wherein
the human IL-21 agonist polypeptide and its masking moiety are fused to different polypeptide chains of the antibody Fc domain or to the different heavy chains of the antibody.

15. The prodrug of claim 13 or 14, wherein the human IL-21 agonist polypeptide and its masking moiety are fused to the C-termini of the two different polypeptide chains of the Fc domain or to the C-termini of the two different heavy chains of the antibody.

16. The prodrug of claim 13 or 14, wherein the human IL-21 agonist polypeptide and its masking moiety are fused to the N-termini of the two different polypeptide chains of the Fc domain or to the N-termini of the two different heavy chains of the antibody.

17. The prodrug of any one of claims 13-16, wherein the carrier moiety is an antibody or an antigen-binding fragment thereof that specifically binds to one or more antigens selected from Guanyl cyclase C (GCC), carbohydrate antigen 19-9 (CA19-9), glycoprotein A33 (gpA33), mucin 1 (MUC1), carcinoembryonic antigen (CEA), insulin-like growth factor 1 receptor (IGF1-R), human epidermal growth factor receptor 2 (HER2), human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGFR1), vascular endothelial growth factor receptor 2 (VEGFR2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), folate receptor alpha (FR-α), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD20, CD22, CD33, CD40, CD56, CD66e, CD70, CD74, CD79b, CD98, CD123, CD138, CD352, CD47, signal-regulatory protein alpha (SIRPα), PD1, Claudin 18.2, Claudin 6, 5T4, BCMA, PD-L1, PD-1, Fibroblast Activation Protein alpha (FAPalpha), the Melanoma-associated Chondroitin Sulfate Proteoglycan (MCSP), and EPCAM.

18. The prodrug of claim 13, wherein the prodrug comprises two polypeptide chains whose amino acid sequences respectively comprise
SEQ ID NOs: 36 and one selected from SEQ ID NO: 101-104,
SEQ ID NOs: 37 and one selected from SEQ ID NO: 101-104,
SEQ ID NOs: 39 and one selected from SEQ ID NO: 105-108,
SEQ ID NOs: 40 and one selected from SEQ ID NO: 105-108,
SEQ ID NOs: 42 and one selected from SEQ ID: 113-116, or
SEQ ID NOs: 43 and one selected from SEQ ID NO: 113-116.

19. The prodrug of claim 13, wherein the carrier moiety is an antibody, wherein the prodrug comprises two identical light chains and two heavy chain polypeptide chains, wherein the light chains comprises an amino acid sequence as shown in SEQ ID NO: 50 or 51, and wherein the first heavy chain polypeptide chain comprises SEQ ID NO: 48, and the second heavy chain polypeptide chain comprises an amino acid sequence selected from SEQ ID NO: 109-112.

20. The prodrug of claim 13, wherein the carrier moiety is an antibody; wherein the prodrug comprises one Fc fusion polypeptide, one light chain and one heavy chain polypeptide chain, wherein the Fc fusion polypeptide comprises an amino acid sequence selected from SEQ ID NO: 101-104, wherein the light chain comprises an amino acid sequence as shown in SEQ ID NO: 50 or 51, and wherein the heavy chain polypeptide chain comprises SEQ ID NO: 48.

21. The prodrug of claim 13, wherein the carrier moiety is an antibody, wherein the prodrug comprises one Fc fusion polypeptide, one light chain and one heavy chain polypeptide chain, wherein the Fc fusion polypeptide comprises an amino acid sequence selected from SEQ ID NO: 36 and 37, wherein the light chain comprises an amino acid sequence as shown in SEQ ID NO: 50 or 51, and wherein the heavy chain polypeptide chain comprises an amino acid sequence selected from SEQ ID NO: 109-112.

22. The prodrug of any of the proceeding claims, wherein the prodrug further comprises an extracellular domain (ECD) of IL-21 receptor, wherein the ECD comprises an amino acid sequence of SEQ ID NO: 128, or at least 95% identical as that of SEQ ID NO: 128.

23. The prodrug of claim 22, wherein the prodrug comprises a light chain of antibody, a first heavy chain polypeptide chain and a second heavy chain polypeptide chain, wherein the light chain comprises an amino acid sequence of SEQ ID NO: 50 or at least 95% identical as SEQ ID NO: 50, the first heavy chain polypeptide chain comprises an amino acid sequence of SEQ ID NO: 117 or 129, or at least 95% identical as that of SEQ ID NO: 117 or 129, and the second heavy chain polypeptide chain with an amino acid sequence selected from SEQ ID NOs: 120, 121, 124, 125, 130, and 131, or an amino acid sequence at least 95% identical as one selected from SEQ ID NOs: 120, 121, 124, 125, 130, or 131.

24. The prodrug of claim 22, wherein the prodrug comprises a light chain of antibody, a first heavy chain polypeptide chain and a second heavy chain polypeptide chain, wherein the light chain comprises an amino acid sequence of SEQ ID NO: 50 or at least 95% identical as SEQ ID NO: 50, the first heavy chain polypeptide chain comprises an amino acid sequence of SEQ ID NO: 118 or at least 95% identical as that of SEQ ID NO: 118, and the second heavy chain polypeptide chain with an amino acid sequence selected from SEQ ID NOs: 122 and 126, or an amino acid sequence at least 95% identical as one selected from SEQ ID NOs: 122 and 126.

25. The prodrug of claim 22, wherein the prodrug comprises a light chain of antibody, a first heavy chain polypeptide chain and a second heavy chain polypeptide chain, wherein the light chain comprises an amino acid sequence of SEQ ID NO: 50 or at least 95% identical as SEQ ID NO: 50, the first heavy chain polypeptide chain comprises an amino acid sequence of SEQ ID NO: 119 or at least 95% identical as that of SEQ ID NO: 119, and the second heavy chain polypeptide chain with an amino acid sequence selected from SEQ ID NOs: 123 and 127, or an amino acid sequence at least 95% identical as one selected from SEQ ID NOs: 123 and 127.

26. A pharmaceutical composition comprising the prodrug of any one of claims 1-25 and a pharmaceutically acceptable excipient.

27. A polynucleotide or polynucleotides encoding the prodrug of any one of claims 1-25.

28. An expression vector or vectors comprising the polynucleotide or polynucleotides of claim 27.

29. A host cell comprising the vector(s) of claim 28.

30. The host cell of claim 29, wherein the gene(s) encoding matriptase, uPA, MMP-2, and/or MMP-9 are knocked out in the host cell.

31. A method of making the prodrug of any one of claims 1-25, comprising
culturing the host cell of claim 29 or 30 under conditions that allow expression of the prodrug, wherein the host cell is a mammalian cell, and
isolating the prodrug.

32. A method of treating a cancer or an infectious disease or stimulating the immune system in a patient in need thereof, comprising administering to the patient a therapeutically effective amount of the pharmaceutical composition of claim 26.

33. A cytokine prodrug of any one of claims 1-25 for use in treating a cancer or an infectious disease or stimulating the immune system in a patient in need thereof.

34. Use of a prodrug of any one of claims 1-25 for the manufacture of a medicament for treating a cancer or an infectious disease or stimulating the immune system in a patient in need thereof.

35. The method of claim 32, the prodrug for use of claim 33, or the use of claim 34, wherein the patient has a virus infection, or a cancer selected from the group consisting of breast cancer, lung cancer, pancreatic cancer, esophageal cancer, medullary thyroid cancer, ovarian cancer, uterine cancer, prostate cancer, testicular cancer, colorectal cancer, and stomach cancer.


SEQUENCE LISTING (135 sequences)

SEQ ID NO: 1 - protein, 133 aa, Homo sapiens [sequence omitted]

SEQ ID NO: 2 - protein, 133 aa, Artificial Sequence
  source: /note=“Description of Artificial Sequence Synthetic polypeptide”
QGQDRHMIRMRQLIDIVAQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 3 - protein, 133 aa, Artificial Sequence
  source: /note=“Description of Artificial Sequence Synthetic polypeptide”
QGQDRHMIRMRQLIDIVKQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 4 - protein, 133 aa, Artificial Sequence
  source: /note=“Description of Artificial Sequence Synthetic polypeptide”
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLRRFKSLLQKMIHQHLSSRTHGSEDS

SEQ ID NO: 5 - protein, 133 aa, Artificial Sequence
  source: /note=“Description of Artificial Sequence Synthetic polypeptide”
QGQDRHMIRMRQLIDIVDKLKNYVNDLVPEFLPAPEDVETNCEWSAFSCFQKAQLKSANTGNNERIINVSIKKLKRKPPSTNAGRRQKHRLTCPSCDSYEKKPPKEFLRRFKSLLQKMIHQHLSSRTHGSEDS

SEQ ID NO: 6 - protein, 213 aa, Homo sapiens
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 7 - protein, 240 aa, Homo sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 8 - protein, 133 aa, Homo sapiens [sequence omitted]

SEQ ID NO: 9 - protein, 113 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 10 - protein, 75 aa, Homo sapiens
ITCPPPMSVEHADIWVKSYSLYSRERYICNSGFKRKAGTSSLTECVLNKATNVAHWTTPSLKCIRDPALVHQRPA

SEQ ID NO: 11 - protein, 6 aa, Artificial Sequence
  source: /note=“Description of Artificial Sequence Synthetic peptide”
GPLGVR

SEQ ID NO: 12 - protein, 7 aa, Artificial Sequence
  source: /note=“Description of Artificial Sequence Synthetic peptide”
PLGMWSR

SEQ ID NO: 13 - protein, 7 aa, Artificial Sequence
  source: /note=“Description of Artificial Sequence Synthetic peptide”
PLGLWAR

SEQ ID NO: 14 - protein, 8 aa, Artificial Sequence
  source: /note=“Description of Artificial Sequence Synthetic peptide”
PQGIAGQR

SEQ ID NO: 15 - protein, 6 aa, Artificial Sequence
  source: /note=“Description of Artificial Sequence Synthetic peptide”
PLGLAG

SEQ ID NO: 16 - protein, 6 aa, Artificial Sequence
  source: /note=“Description of Artificial Sequence Synthetic peptide”
LALGPR

SEQ ID NO: 17 - protein, 10 aa, Artificial Sequence
  source: /note=“Description of Artificial Sequence Synthetic peptide”
GGPLGMLSQS

SEQ ID NO: 18 - protein, 9 aa, Artificial Sequence
  source: /note=“Description of Artificial Sequence Synthetic peptide”
GGGGRRGGS

SEQ ID NO: 19 - protein, 8 aa, Artificial Sequence
  source: /note=“Description of Artificial Sequence Synthetic peptide”
TGRGPSWV

SEQ ID NO: 20 - protein, 8 aa, Artificial Sequence
  source: /note=“Description of Artificial Sequence Synthetic peptide”
SARGPSRW

SEQ ID NO: 21 - protein, 8 aa, Artificial Sequence
  source: /note=“Description of Artificial Sequence Synthetic peptide”
TARGPSFK

SEQ ID NO: 22 - protein, 7 aa, Artificial Sequence
  source: /note=“Description of Artificial Sequence Synthetic peptide”
TARGPSW

SEQ ID NO: 23 - protein, 8 aa, Artificial Sequence
  source: /note=“Description of Artificial Sequence Synthetic peptide”
GGWHTGRN

SEQ ID NO: 24 - protein, 8 aa, Artificial Sequence
  source: /note=“Description of Artificial Sequence Synthetic peptide”
HTGRSGAL

SEQ ID NO: 25 - protein, 8 aa, Artificial Sequence
  source: /note=“Description of Artificial Sequence Synthetic peptide”
PLTGRSGG

SEQ ID NO: 26 - protein, 7 aa, Artificial Sequence
  source: /note=“Description of Artificial Sequence Synthetic peptide”
LTGRSGA

SEQ ID NO: 27 - protein, 76 aa, Homo sapiens
TNDAEDCCLSVTQKPIPGYIVRNFHYLLIKDGCRVPAVVFTTLRGRQLCAPPDQPWVERIIQRLQRTSAKMKRRSS

SEQ ID NO: 28 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 29 - protein, 5 aa, Artificial Sequence
  source: /note=“Description of Artificial Sequence Synthetic peptide”
GGGGS

SEQ ID NO: 30 - protein, 10 aa, Artificial Sequence
  source: /note=“Description of Artificial Sequence Synthetic peptide”
GGGGSGGGGS

SEQ ID NO: 31 - protein, 15 aa, Artificial Sequence
  source: /note=“Description of Artificial Sequence Synthetic peptide”
GGGGSGGGGSGGGGS

SEQ ID NO: 32 - protein, 20 aa, Artificial Sequence
  source: /note=“Description of Artificial Sequence Synthetic peptide”
  VARIANT (10)..(10): /replace=“Asn”
  SITE (1)..(20): /note=“Variant residues given in the sequence have no preference with respect to those in the annotations for variant positions”
GGGGSGGGGAGGGGSGGGGS

SEQ ID NO: 33 - protein, 20 aa, Artificial Sequence
  source: /note=“Description of Artificial Sequence Synthetic peptide”
  VARIANT (10)..(10): /replace=“Ala” or “Asn”
  VARIANT (15)..(15): /replace=“Asn”
  SITE (1)..(20): /note=“Variant residues given in the sequence have no preference with respect to those in the annotations for variant positions”
GGGGSGGGGSGGGGAGGGGS

SEQ ID NO: 34 - protein, 227 aa, Unknown
  source: /note=“Description of Unknown IgG1 Fc sequence”
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 230 aa, Unknown
  source: /note=“Description of Unknown IgG4 Fc sequence”
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 385 aa, Artificial Sequence
  source: /note=“Description of Artificial Sequence Synthetic polypeptide”
  SITE (228)..(252): /note=“This region may encompass 1-5 ‘Gly Gly Gly Gly Ser’ repeating units or may be absent in its entirety”
DKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 37 - protein, 385 aa, Artificial Sequence
  source: /note=“Description of Artificial Sequence Synthetic polypeptide”
  SITE (228)..(252): /note=“This region may encompass 1-5 ‘Gly Gly Gly Gly Ser’ repeating units or may be absent in its entirety”
DKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGGGGSQGQDRHMIRMRQLIDIVDKLKNYVNDLVPEFLPAPEDVETNCEWSAFSCFQKAQLKSANTGNNERIINVSIKKLKRKPPSTNAGRRQKHRLTCPSCDSYEKKPPKEFLRRFKSLLQKMIHQHLSSRTHGSEDS

SEQ ID NO: 38 - protein, 461 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 39 - protein, 385 aa, Artificial Sequence
  source: /note=“Description of Artificial Sequence Synthetic polypeptide”
  SITE (134)..(158): /note=“This region may encompass 1-5 ‘Gly Gly Gly Gly Ser’ repeating units or may be absent in its entirety”
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 385 aa, Artificial Sequence
  source: /note=“Description of Artificial Sequence Synthetic polypeptide”
  SITE (134)..(158): /note=“This region may encompass 1-5 ‘Gly Gly Gly Gly Ser’ repeating units or may be absent in its entirety”
QGQDRHMIRMRQLIDIVDKLKNYVNDLVPEFLPAPEDVETNCEWSAFSCFQKAQLKSANTGNNERIINVSIKKLKRKPPSTNAGRRQKHRLTCPSCDSYEKKPPKEFLRRFKSLLQKMIHQHLSSRTHGSEDSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 461 aa, Artificial Sequence
  source: /note=“Description of Artificial Sequence Synthetic polypeptide”
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGGGGSGGGGSGPLGVRGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 42 - protein, 623 aa, Artificial Sequence
  source: /note=“Description of Artificial Sequence Synthetic polypeptide”
  VARIANT (108)..(108): /replace=“Glu”
  SITE (114)..(138): /note=“This region may encompass 1-5 ‘Gly Gly Gly Gly Ser’ repeating units or may be absent in its entirety”
  SITE (214)..(238): /note=“This region may encompass 1-5 ‘Gly Gly Gly Gly Ser’ repeating units or may be absent in its entirety”
  SITE (466)..(490): /note=“This region may encompass 1-5 ‘Gly Gly Gly Gly Ser’ repeating units or may be absent in its entirety”
  SITE (1)..(623): /note=“Variant residues given in the sequence have no preference with respect to those in the annotations for variant positions”
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTGGGGSGGGGSGGGGSGGGGSGGGGSITCPPPMSVEHADIWVKSYSLYSRERYICNSGFKRKAGTSSLTECVLNKATNVAHWTTPSLKCIRDPALVHQRPAGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 43 - protein, 543 aa, Artificial Sequence
  source: /note=“Description of Artificial Sequence Synthetic polypeptide”
  VARIANT (126)..(126): /replace=“Ala” or “Trp” or “His” or “Glu”
  SITE (134)..(158): /note=“This region may encompass 1-5 ‘Gly Gly Gly Gly Ser’ repeating units or may be absent in its entirety”
  SITE (386)..(410): /note=“This region may encompass 1-5 ‘Gly Gly Gly Gly Ser’ repeating units or may be absent in its entirety”
  SITE (1)..(543): /note=“Variant residues given in the sequence have no preference with respect to those in the annotations for variant positions”
APASSSTKKTQLQLEHLLLDLQMILNGINNYKNPKLTSMLTKKFAMPKKATELKHLQCLEEALKPLEEVLNLTQSKNFHLRPRDLISNINVIVLELKGSETTFMCEYADETATIVEFLNRWITFSQSIISTLTGGGGSGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGGGGSQGQDRHMIRMRQLIDIVDKLKNYVNDLVPEFLPAPEDVETNCEWSAFSCFQKAQLKSANTGNNERIINVSIKKLKRKPPSTNAGRRQKHRLTCPSCDSYEKKPPKEFLRRFKSLLQKMIHQHLSSRTHGSEDS

SEQ ID NO: 44 - protein, 696 aa, Artificial Sequence
  source: /note=“Description of Artificial Sequence Synthetic polypeptide”
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGPLGVRGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 45 - protein, 623 aa, Artificial Sequence
  source: /note=“Description of Artificial Sequence Synthetic polypeptide”
  SITE (134)..(158): /note=“This region may encompass 1-5 ‘Gly Gly Gly Gly Ser’ repeating units or may be absent in its entirety”
  SITE (386)..(410): /note=“This region may encompass 1-5 ‘Gly Gly Gly Gly Ser’ repeating units or may be absent in its entirety”
  SITE (486)..(510): /note=“This region may encompass 1-5 ‘Gly Gly Gly Gly Ser’ repeating units or may be absent in its entirety”
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGGGGSITCPPPMSVEHADIWVKSYSLYSRERYICNSGFKRKAGTSSLTECVLNKATNVAHWTTPSLKCIRDPALVHQRPAGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINT

SEQ ID NO: 46 - protein, 543 aa, Artificial Sequence
  source: /note=“Description of Artificial Sequence Synthetic polypeptide”
  SITE (134)..(158): /note=“This region may encompass 1-5 ‘Gly Gly Gly Gly Ser’ repeating units or may be absent in its entirety”
  SITE (386)..(410): /note=“This region may encompass 1-5 ‘Gly Gly Gly Gly Ser’ repeating units or may be absent in its entirety”
QGQDRHMIRMRQLIDIVDKLKNYVNDLVPEFLPAPEDVETNCEWSAFSCFQKAQLKSANTGNNERIINVSIKKLKRKPPSTNAGRRQKHRLTCPSCDSYEKKPPKEFLRRFKSLLQKMIHQHLSSRTHGSEDSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSGGGGSGGGGSAPASSSTKKTQLQLEHLLLDLQMILNGINNYKNPKLTSMLTKKFAMPKKATELKHLQCLEEALKPLEEVLNLTQSKNFHLRPRDLISNINVIVLELKGSETTFMCEYADETATIVEFLNRWITFSQSIISTLT

SEQ ID NO: 47 - protein, 696 aa, Artificial Sequence
  source: /note=“Description of Artificial Sequence Synthetic polypeptide”
CPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGGGGSGGGGSGPLGVRGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 48 - protein, 588 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 49 - protein, 674 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGPLGVRGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 50 - protein, 214 aa, Artificial Sequence
  source: /note=“Description of Artificial Sequence Synthetic polypeptide”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 362 aa, Artificial Sequence
  source: /note=“Description of Artificial Sequence Synthetic polypeptide”
  VARIANT (317)..(317): /replace=“Ala”
  VARIANT (355)..(355): /replace=“His” or “Trp” or “Ala”
  SITE (1)..(362): /note=“Variant residues given in the sequence have no preference with respect to those in the annotations for variant positions”
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSAPASSSTKKTQLQLEHLLLDLQMILNGINNYKNPKLTSMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 52 - protein, 214 aa, Artificial Sequence
  source: /note=“Description of Artificial Sequence Synthetic polypeptide”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 451 aa, Artificial Sequence
  source: /note=“Description of Artificial Sequence Synthetic polypeptide”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 213 aa, Artificial Sequence
  source: /note=“Description of Artificial Sequence Synthetic polypeptide”
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 451 aa, Artificial Sequence
  source: /note=“Description of Artificial Sequence Synthetic polypeptide”
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 218 aa, Artificial Sequence
  source: /note=“Description of Artificial Sequence Synthetic polypeptide”
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 446 aa, Artificial Sequence
  source: /note=“Description of Artificial Sequence Synthetic polypeptide”
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 214 aa, Artificial Sequence
  source: /note=“Description of Artificial Sequence Synthetic polypeptide”
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 449 aa, Artificial Sequence
  source: /note=“Description of Artificial Sequence Synthetic polypeptide”
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 214 aa, Artificial Sequence
  source: /note=“Description of Artificial Sequence Synthetic polypeptide”
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 385 aa, Artificial Sequence
  source: /note=“Description of Artificial Sequence Synthetic polypeptide”
GHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDERKCCVECPAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 62 - protein, 219 aa, Artificial Sequence
  source: /note=“Description of Artificial Sequence Synthetic polypeptide”
DIVMTQAAPSVPVTPGESVSISCRSSKSLLHSNGNTYLYWFLQRPGQSPQVLIYRMSNLASGVPDRFSGSGSGTAFTLRIRRVEAEDVGVYYCMQNLEYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 444 aa, Artificial Sequence
  source: /note=“Description of Artificial Sequence Synthetic polypeptide”
QVQLQQSGPELVKSGASVKMSCKASGNTLKDDHVHWVKQRPGQGLEWIGWIYPGGGRTRYNEKFKGKTTLTADKPSSTVNMLLSSLTSEDSAIYFCTNLVFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 219 aa, Artificial Sequence
  source: /note=“Description of Artificial Sequence Synthetic polypeptide”
DVVMTQSPLSLPVTPGEPASISCRSSQSLVHSNANTYLHWYLQKPGQSPQLLIYKVSNRFSGVPDRFSGSGSGTDFTLKISRVEAEDVGVYYCSQNTHVPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 445 aa, Artificial Sequence
  source: /note=“Description of Artificial Sequence Synthetic polypeptide”
QVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 220 aa, Artificial Sequence
  source: /note=“Description of Artificial Sequence Synthetic polypeptide”
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 448 aa, Artificial Sequence
  source: /note=“Description of Artificial Sequence Synthetic polypeptide”
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 11 aa, Artificial Sequence
  source: /note=“Description of Artificial Sequence Synthetic peptide”
KASQDVSIAVA

SEQ ID NO: 69 - protein, 7 aa, Artificial Sequence
  source: /note=“Description of Artificial Sequence Synthetic peptide”
SASYRYT

SEQ ID NO: 70 - protein, 9 aa, Artificial Sequence
  source: /note=“Description of Artificial Sequence Synthetic peptide”
QQHYITPLT

SEQ ID NO: 71 - protein, 5 aa, Artificial Sequence
  source: /note=“Description of Artificial Sequence Synthetic peptide”
NYGMN

SEQ ID NO: 72 - protein, 17 aa, Artificial Sequence
  source: /note=“Description of Artificial Sequence Synthetic peptide”
WINTYTGEPTYTDDFKG

SEQ ID NO: 73 - protein, 12 aa, Artificial Sequence
  source: /note=“Description of Artificial Sequence Synthetic peptide”
GGFGSSYWYFDV

SEQ ID NO: 74 - protein, 10 aa, Artificial Sequence
  source: /note=“Description of Artificial Sequence Synthetic peptide”
SASSSVSYMH

SEQ ID NO: 75 - protein, 7 aa, Artificial Sequence
  source: /note=“Description of Artificial Sequence Synthetic peptide”
DTSKLAS

SEQ ID NO: 76 - protein, 9 aa, Artificial Sequence
  source: /note=“Description of Artificial Sequence Synthetic peptide”
QQWSGYPLT

SEQ ID NO: 77 - protein, 5 aa, Artificial Sequence
  source: /note=“Description of Artificial Sequence Synthetic peptide”
GYTMN

SEQ ID NO: 78 - protein, 17 aa, Artificial Sequence
  source: /note=“Description of Artificial Sequence Synthetic peptide”
LITPYNGASSYNQKFRG

SEQ ID NO: 79 - protein, 10 aa, Artificial Sequence
  source: /note=“Description of Artificial Sequence Synthetic peptide”
GGYDGRGFDY

SEQ ID NO: 80 - protein, 213 aa, Artificial Sequence
  source: /note=“Description of Artificial Sequence Synthetic polypeptide”
QIVLTQSPATLSLSPGERATLSCSASSGVNFMHWYQQKPGQAPRRLIFDTSKLASGIPARFSGSGSGTDYTLTISSLEPEDFAVYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 213 aa, Artificial Sequence
  source: /note=“Description of Artificial Sequence Synthetic polypeptide”
QIVLTQSPATLSLSPGERATLSCSASSGVNFMHWYQQKPGQAPKRLIFDTSKLASGVPARFSGSGSGTDYTLTISSLEPEDFAVYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - protein, 447 aa, Artificial Sequence
  source: /note=“Description of Artificial Sequence Synthetic polypeptide”
QVQLVQSGAEVKKPGASVKVSCKASGYTFTNNGINWLRQAPGQGLEWMGEIYPRSTNTLYAQKFQGRVTITADRSSNTAYMELSSLRSEDTAVYFCARTLTAPFAFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 113 aa, Artificial Sequence
  source: /note=“Description of Artificial Sequence Synthetic polypeptide”
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 84 - protein, 124 aa, Artificial Sequence
  source: /note=“Description of Artificial Sequence Synthetic polypeptide”
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 85 - protein, 120 aa, Artificial Sequence
  source: /note=“Description of Artificial Sequence Synthetic polypeptide”
QVQLVQSGAEVKKPGASVKVSCKASGYSFTGYYMHWVKQSPGQGLEWIGRINPNNGVTLYNQKFKDRVTMTRDTSISTAYMELSRLRSDDTAVYYCARSTMITNYVMDYWGQGTLWTVSS

SEQ ID NO: 86 - protein, 120 aa, Artificial Sequence
  source: /note=“Description of Artificial Sequence Synthetic polypeptide”
QVQLVQSGAEVKKPGASVKVSCKASGYSFTGYYMHWVRQAPGQGLEWMGRINPNNGVTLYNQKFKDRVTMTRDTSISTAYMELSRLRSDDTAVYYCARSTMITNYVMDYWGQGTLVTVSS

SEQ ID NO: 87 - protein, 107 aa, Artificial Sequence
  source: /note=“Description of Artificial Sequence Synthetic polypeptide”
DIVMTQSPDSLAVSLGERATINCKASQSVSNDVAWYQQKPGQSPKLLISYTSSRYAGVPDRFSGSGSGTDFTLTISSLQAEDVAVYFCQQDYNSPPTFGGGTKLEIK

SEQ ID NO: 88 - protein, 107 aa, Artificial Sequence
  source: /note=“Description of Artificial Sequence Synthetic polypeptide”
DIVMTQSPDSLAVSLGERATINCKASQSVSNDVAWYQQKPGQPPKLLIYYTSSRYAGVPDRFSGSGSGTDFTLTISSLQAEDVAVYYCQQDYNSPPTFGGGTKLEIK

SEQ ID NO: 89 - protein, 214 aa, Artificial Sequence
  source: /note=“Description of Artificial Sequence Synthetic polypeptide”
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 448 aa, Artificial Sequence
  source: /note=“Description of Artificial Sequence Synthetic polypeptide”
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 440 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - protein, 218 aa, Artificial Sequence
  source: /note=“Description of Artificial Sequence Synthetic polypeptide”
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 447 aa, Artificial Sequence
  source: /note=“Description of Artificial Sequence Synthetic polypeptide”
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 375 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 95 - protein, 375 aa, Artificial Sequence
  source: /note=“Description of Artificial Sequence Synthetic polypeptide”
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSQGQDRHMIRMRQLIDIVDKLKNYVNDLVPEFLPAPEDVETNCEWSAFSCFQKAQLKSANTGNNERIINVSIKKLKRKPPSTNAGRRQKHRLTCPSCDSYEKKPPKEFLRRFKSLLQKMIHQHLSSRTHGSEDS

SEQ ID NO: 96 - protein, 227 aa, Artificial Sequence
  source: /note=“Description of Artificial Sequence Synthetic polypeptide”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97 - protein, 136 aa, Artificial Sequence
  source: /note=“Description of Artificial Sequence Synthetic polypeptide”
MKHLWFFLLLVAAPRWVLSQVQLQESGPGLVKPSETLSLTCTVSGGSISSDFWGWIRQPPGKGLEWIGYISSRGSTNYNPSLKRRVTISVDTSRNQFSLKLSSVTAADTAVYYCARSAGVTDFDFWGQGTLVTVSS

SEQ ID NO: 98 - protein, 129 aa, Artificial Sequence
  source: /note=“Description of Artificial Sequence Synthetic polypeptide”
MDMMVPAQLLGLLLLWFPGSRCDIQMTQSPSSVSASVGDRVTITCRASQGISSWLAWYQHKPGKAPKLLIYVASSLQSGVPSRFSGSGSGTDFTLTISSLQPEDFATYYCQQANSFPLTFGGGTKVEIK

SEQ ID NO: 99 - protein, 145 aa, Artificial Sequence
  source: /note=“Description of Artificial Sequence Synthetic polypeptide”
MEFGLSWVFLVALLRGVQCQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 100 - protein, 126 aa, Artificial Sequence
  source: /note=“Description of Artificial Sequence Synthetic polypeptide”
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIK

SEQ ID NO: 101 - protein, 497 aa, Artificial Sequence
  source: /note=“Description of Artificial Sequence Synthetic polypeptide”
  SITE (366)..(390): /note=“This region may encompass 1-5 ‘Gly Gly Gly Gly Ser’ repeating units”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSQVQLQESGPGLVKPSETLSLTCTVSGGSISSDFWGWIRQPPGKGLEWIGYISSRGSTNYNPSLKRRVTISVDTSRNQFSLKLSSVTAADTAVYYCARSAGVTDFDFWGQGTLVTVSSGGGGSGGGGSGGGGSGGGGSGGGGSDIQMTQSPSSVSASVGDRVTITCRASQGISSWLAWYQHKPGKAPKLLIYVASSLQSGVPSRFSGSGSGTDFTLTISSLQPEDFATYYCQQANSFPLTFGGGTKVEIK

SEQ ID NO: 102 - protein, 497 aa, Artificial Sequence
  source: /note=“Description of Artificial Sequence Synthetic polypeptide”
  SITE (356)..(380): /note=“This region may encompass 1-5 ‘Gly Gly Gly Gly Ser’ repeating units”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSDIQMTQSPSSVSASVGDRVTITCRASQGISSWLAWYQHKPGKAPKLLIYVASSLQSGVPSRFSGSGSGTDFTLTISSLQPEDFATYYCQQANSFPLTFGGGTKVEIKGGGGSGGGGSGGGGSGGGGSGGGGSQVQLQESGPGLVKPSETLSLTCTVSGGSISSDFWGWIRQPPGKGLEWIGYISSRGSTNYNPSLKRRVTISVDTSRNQFSLKLSSVTAADTAVYYCARSAGVTDFDFWGQGTLVTVSS

SEQ ID NO: 103 - protein, 505 aa, Artificial Sequence
  source: /note=“Description of Artificial Sequence Synthetic polypeptide”
  SITE (375)..(399): /note=“This region may encompass 1-5 ‘Gly Gly Gly Gly Ser’ repeating units”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIK

SEQ ID NO: 104 - protein, 505 aa, Artificial Sequence
  source: /note=“Description of Artificial Sequence Synthetic polypeptide”
  SITE (355)..(379): /note=“This region may encompass 1-5 ‘Gly Gly Gly Gly Ser’ repeating units”
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 105 - protein, 497 aa, Artificial Sequence
  source: /note=“Description of Artificial Sequence Synthetic polypeptide”
  SITE (118)..(142): /note=“This region may encompass 1-5 ‘Gly Gly Gly Gly Ser’ repeating units”
QVQLQESGPGLVKPSETLSLTCTVSGGSISSDFWGWIRQPPGKGLEWIGYISSRGSTNYNPSLKRRVTISVDTSRNQFSLKLSSVTAADTAVYYCARSAGVTDFDFWGQGTLVTVSSGGGGSGGGGSGGGGSGGGGSGGGGSDIQMTQSPSSVSASVGDRVTITCRASQGISSWLAWYQHKPGKAPKLLIYVASSLQSGVPSRFSGSGSGTDFTLTISSLQPEDFATYYCQQANSFPLTFGGGTKVEIKGGGGSGGGGSGPLGVRGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 106 - protein, 497 aa, Artificial Sequence
  source: /note=“Description of Artificial Sequence Synthetic polypeptide”
  SITE (108)..(132): /note=“This region may encompass 1-5 ‘Gly Gly Gly Gly Ser’ repeating units”
DIQMTQSPSSVSASVGDRVTITCRASQGISSWLAWYQHKPGKAPKLLIYVASSLQSGVPSRFSGSGSGTDFTLTISSLQPEDFATYYCQQANSFPLTFGGGTKVEIKGGGGSGGGGSGGGGSGGGGSGGGGSQVQLQESGPGLVKPSETLSLTCTVSGGSISSDFWGWIRQPPGKGLEWIGYISSRGSTNYNPSLKRRVTISVDTSRNQFSLKLSSVTAADTAVYYCARSAGVTDFDFWGQGTLVTVSSGGGGSGGGGSGPLGVRGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 107 - protein, 505 aa, Artificial Sequence
  source: /note=“Description of Artificial Sequence Synthetic polypeptide”
  SITE (127)..(151): /note=“This region may encompass 1-5 ‘Gly Gly Gly Gly Ser’ repeating units”
QVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGPLGVRGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 108 - protein, 505 aa, Artificial Sequence
  source: /note=“Description of Artificial Sequence Synthetic polypeptide”
  SITE (107)..(131): /note=“This region may encompass 1-5 ‘Gly Gly Gly Gly Ser’ repeating units”
EIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSGGGGSGGGGSGPLGVRGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 109 - protein, 710 aa, Artificial Sequence
  source: /note=“Description of Artificial Sequence Synthetic polypeptide”
  SITE (579)..(603): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGPLGVRGGGGSQVQLQESGPGLVKPSETLSLTCTVSGGSISSDFWGWIRQPPGKGLEWIGYISSRGSTNYNPSLKRRVTISVDTSRNQFSLKLSSVTAADTAVYYCARSAGVTDFDFWGQGTLVTVSSGGGGSGGGGSGGGGSGGGGSGGGGSDIQMTQSPSSVSASVGDRVTITCRASQGISSWLAWYQHKPGKAPKLLIYVASSLQSGVPSRFSGSGSGTDFTLTISSLQPEDFATYYCQQANSFPLTFGGGTKVEIK

SEQ ID NO: 110 - protein, 710 aa, Artificial Sequence
  source: /note=“Description of Artificial Sequence Synthetic polypeptide”
  SITE (569)..(593): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGPLGVRGGGGSDIQMTQSPSSVSASVGDRVTITCRASQGISSWLAWYQHKPGKAPKLLIYVASSLQSGVPSRFSGSGSGTDFTLTISSLQPEDFATYYCQQANSFPLTFGGGTKVEIKGGGGSGGGGSGGGGSGGGGSGGGGSQVQLQESGPGLVKPSETLSLTCTVSGGSISSDFWGWIRQPPGKGLEWIGYISSRGSTNYNPSLKRRVTISVDTSRNQFSLKLSSVTAADTAVYYCARSAGVTDFDFWGQGTLVTVSS

SEQ ID NO: 111 - protein, 718 aa, Artificial Sequence
  source: /note=“Description of Artificial Sequence Synthetic polypeptide”
  SITE (588)..(612): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGPLGVRGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIK

SEQ ID NO: 112 - protein, 718 aa, Artificial Sequence
  source: /note=“Description of Artificial Sequence Synthetic polypeptide”
  SITE (568)..(592): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGPLGVRGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 113 - protein, 732 aa, Artificial Sequence
  source: /note=“Description of Artificial Sequence Synthetic polypeptide”
  SITE (601)..(625): /note=“This region may encompass 1-5 ‘Gly Gly Gly Gly Ser’ repeating units”
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGPLGVRGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSQVQLQESGPGLVKPSETLSLTCTVSGGSISSDFWGWIRQPPGKGLEWIGYISSRGSTNYNPSLKRRVTISVDTSRNQFSLKLSSVTAADTAVYYCARSAGVTDFDFWGQGTLVTVSSGGGGSGGGGSGGGGSGGGGSGGGGSDIQMTQSPSSVSASVGDRVTITCRASQGISSWLAWYQHKPGKAPKLLIYVASSLQSGVPSRFSGSGSGTDFTLTISSLQPEDFATYYCQQANSFPLTFGGGTKVEIK

SEQ ID NO: 114 - protein, 732 aa, Artificial Sequence
  source: /note=“Description of Artificial Sequence Synthetic polypeptide”
  SITE (591)..(615): /note=“This region may encompass 1-5 ‘Gly Gly Gly Gly Ser’ repeating units”
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGPLGVRGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSDIQMTQSPSSVSASVGDRVTITCRASQGISSWLAWYQHKPGKAPKLLIYVASSLQSGVPSRFSGSGSGTDFTLTISSLQPEDFATYYCQQANSFPLTFGGGTKVEIKGGGGSGGGGSGGGGSGGGGSGGGGSQVQLQESGPGLVKPSETLSLTCTVSGGSISSDFWGWIRQPPGKGLEWIGYISSRGSTNYNPSLKRRVTISVDTSRNQFSLKLSSVTAADTAVYYCARSAGVTDFDFWGQGTLVTVSS

SEQ ID NO: 115 - protein, 740 aa, Artificial Sequence
  source: /note=“Description of Artificial Sequence Synthetic polypeptide”
  SITE (610)..(634): /note=“This region may encompass 1-5 ‘Gly Gly Gly Gly Ser’ repeating units”
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGPLGVRGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIK

SEQ ID NO: 116 - protein, 740 aa, Artificial Sequence
  source: /note=“Description of Artificial Sequence Synthetic polypeptide”
  SITE (590)..(614): /note=“This region may encompass 1-5 ‘Gly Gly Gly Gly Ser’ repeating units”
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTGGGGSGGGGSGPLGVRGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGAGGGGSGGGGSGPLGVRGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 117 - protein, 588 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 118 - protein, 893 aa, Artificial Sequence
  source: /note=“Description of Artificial Sequence Synthetic polypeptide”
  SITE (589)..(613): /note=“This region may encompass 1-5 ‘Gly Gly Gly Gly Ser’ repeating units”
  SITE (622)..(646): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSGGGGSGGGGSRQARVVNGGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 119 - protein, 859 aa, Artificial Sequence
  source: /note=“Description of Artificial Sequence Synthetic polypeptide”
  SITE (589)..(613): /note=“This region may encompass 1-5 ‘Gly Gly Gly Gly Ser’ repeating units”
  SITE (622)..(646): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSGGGGSGGGGSRQARVVNGGGGGSGGGGSGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 120 - protein, 979 aa, Artificial Sequence
  source: /note=“Description of Artificial Sequence Synthetic polypeptide”
  SITE (675)..(699): /note=“This region may encompass 1-5 ‘Gly Gly Gly Gly Ser’ repeating units”
  SITE (708)..(732): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGGGGSGGGGSGGGGSGGGGSGGGGSRQARVVNGGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 121 - protein, 1014 aa, Artificial Sequence
  source: /note=“Description of Artificial Sequence Synthetic polypeptide”
  SITE (441)..(465): /note=“This region may encompass 1-5 ‘Gly Gly Gly Gly Ser’ repeating units”
  SITE (474)..(498): /note=“This region may encompass 1-5 ‘Gly Gly Gly Gly Ser’ repeating units”
  SITE (746)..(770): /note=“This region may encompass 1-5 ‘Gly Gly Gly Gly Ser’ repeating units”
  SITE (777)..(801): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSRQARVVNGGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSGGGGSGGGGSGGGGSGGGGSGGGGSGPLGVRGGGGSGGGGSGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 122 - protein, 674 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 123 - protein, 708 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGPLGVRGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 124 - protein, 983 aa, Artificial Sequence
  source: /note=“Description of Artificial Sequence Synthetic polypeptide”
  SITE (679)..(703): /note=“This region may encompass 1-5 ‘Gly Gly Gly Gly Ser’ repeating units”
  SITE (712)..(736): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGPLGMLSQSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGGGGSGGGGSGGGGSGGGGSGGGGSRQARVVNGGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 125 - protein, 1018 aa, Artificial Sequence
  source: /note=“Description of Artificial Sequence Synthetic polypeptide”
  SITE (441)..(465): /note=“This region may encompass 1-5 ‘Gly Gly Gly Gly Ser’ repeating units”
  SITE (474)..(498): /note=“This region may encompass 1-5 ‘Gly Gly Gly Gly Ser’ repeating units”
  SITE (746)..(770): /note=“This region may encompass 1-5 ‘Gly Gly Gly Gly Ser’ repeating units”
  SITE (781)..(805): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSRQARVVNGGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSSGGGGSGGGGSGGGGSGGGGSGGGGSGGPLGMLSQSGGGGSGGGGSGGGGSGGGGSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 126 - protein, 678 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGPLGMLSQSGGGGSCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKE

SEQ ID NO: 127 - protein, 712 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGPLGMLSQSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 128 - protein, 213 aa, Homo sapiens; identical to SEQ ID NO: 6

SEQ ID NO: 129 - protein, 588 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSQGQDRHMIRMRQLIDIVDQLKNYVNDLVPEFLPAPEDVETNCEWSAFSCFQKAQLKSANTGNNERIINVSIKALKRKPPSTNAGRRQKHRLTCPSCDSYEKKPPKEFLERFKSLLQKMIHQHLSSRTHGSEDS

SEQ ID NO: 130 - protein, 709 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSAAGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 131 - protein, 745 aa, Artificial Sequence
  source: /note=“Description of Artificial Sequence Synthetic polypeptide”
  SITE (441)..(465): /note=“This region may encompass 1-5 ‘Gly Gly Gly Gly Ser’ repeating units”
  SITE (474)..(498): /note=“This region may encompass 1-5 ‘Gly Gly Gly Gly Ser’ repeating units”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAGGGGSGGGGSGGGGSGGGGSGGGGSRQARVVNGGGGGSGGGGSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 132 - protein, 22 aa, Artificial Sequence
  source: /note=“Description of Artificial Sequence Synthetic peptide”
GGGGSGGGGSAAGGGGSGGGGS

SEQ ID NO: 133 - protein, 708 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGPLGVRGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSWTFGQGTKVEIKGGGGSGGGGSGGGGSQVQLVESGGGVVQPGRSLRLSCAASGFTFSSYGMHWVRQAPGKGLEWVAFIWYDGSDKYYADSVKGRFTISRDNSKNTLYLQMNSLRAEDTAVYYCARDGDSSDWYGDYYFGMDVWGQGTTVTVSS

SEQ ID NO: 134 - protein, 440 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 135 - protein, 578 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGAGGGGSQGQDRHMIEMRQLIDIVDQLKNYVNDLVPEFLPAPEDVETNCEWSAFSCFQKAQLKSANTGNNERIINVSIKKLKAKPPSTNAGRRQKHRLTCPSCDSYEKKPPKEFLERFKSLLQKMIHQHLSSRTHGSEDS